(19) United States
(12) Patent Application Publication
(10) Pub. No.: US 2025/0186147 A1
(43) Pub. Date: Jun. 12, 2025

(54) MANUAL JAW GRIP RELEASE DETECTION

(71) Applicant: Intuitive Surgical Operations, Inc., Sunnyvale, CA (US)

(72) Inventors: Matthew TIMES, Milford, CT (US); Gabriel F. BRISSON, Livermore, CA (US); Wesley Chung JOE, Fremont, CA (US); Paul A. MILLMAN, Denver, CO (US)

(73) Assignee: Intuitive Surgical Operations, Inc., Sunnyvale, CA (US)

(21) Appl. No.: 18/846,464

(22) PCT Filed: Mar. 7, 2023
(86) PCT No.: PCT/US2023/014696

§ 371 Date: Sep. 12, 2024

Related U.S. Application Data
(60) Provisional application No. 63/320,179, filed on Mar. 15, 2022.

(51) Int. Cl. A61B 34/35

(52) U.S. Cl. CPC A61B 34/35

(57) ABSTRACT

A surgical system includes an actuator system and a control system operatively coupled to the actuator system. The actuator system is operable to drive a first movable operating component and a second movable operating component. The control system includes a memory and instructions stored in the memory. The instructions cause the control system to perform actions including commanding the actuator system to operate in a first control mode, receiving a first signal containing a first kinematic parameter value of the first movable operating component, receiving a second signal containing a second kinematic parameter value of the second movable operating component, determining a compared value derived from both the first kinematic parameter value and the second kinematic parameter value, and causing the control system to operate the actuator system in an instrument release mode on a condition in which the compared value is within a defined value range.

### CROSS-REFERENCE TO RELATED APPLICATIONS

This application claims benefit of priority to U.S. Provisional Application Ser. No. 63/320,179, entitled “Manual Jaw Grip Release Detection,” filed Mar. 15, 2022, which is incorporated herein by reference in its entirety.

### BACKGROUND

The embodiments described herein relate to medical devices, and more specifically to endoscopic tools. More particularly, the embodiments described herein relate to medical devices that include a manual drive that drives a tool drive member and methods for detecting when the manual drive has been engaged.

Known techniques for Minimally Invasive Surgery (MIS) employ instruments to manipulate tissue that can be either manually controlled or controlled via computer-assisted teleoperation. Many known MIS instruments include a therapeutic or diagnostic end effector (e.g., forceps, a cutting tool, or a cauterizing tool) mounted on a wrist mechanism at the distal end of a shaft. During an MIS procedure, the end effector, wrist mechanism, and the distal end of the shaft are inserted into a small incision or a natural orifice of a patient to position the end effector at a work site within the patient's body.

To enable the desired movement of the distal wrist mechanism and end effector, known instruments include motors, capstans, and cables. The cables extend through the shaft that connects the wrist mechanism to a mechanical structure. For teleoperated systems, the mechanical structure is typically motor driven and is operably coupled to a computer processing system to provide a user interface for a clinical user (e.g., a surgeon) to control the instrument as a whole, as well as the instrument's components and functions. Some teleoperated systems include a manual control separate from the motor driven aspects allowing a user some level of manual interaction with the medical device. Some known manual controls allow the user to override the motor driven control to manually open the jaws of an instrument (e.g., when a system fault occurs or during a power outage).

Patients benefit from continual efforts to improve the effectiveness of MIS methods and devices and, in particular, the manual interactions with the medical device. For example, making the manual controls easy to operate (e.g., without actively engaging specific modes of operation to use the manual controls) allows for a surgeon to have more limited knowledge for the manipulation of the device simplifying the surgical environment. In particular, automating the switching of operation modes on the device for instances of use of the manual control on a surgical instrument decreases unnecessary steps and simplifies the operation of the manual control. For example, some known manual controls require the surgeon to actively change operation modes on the device before using the manual controls. Thus, actuation of the manual control is complex. Producing medical devices that implement the clinically desired functions for minimally invasive procedures can be challenging. For example, cost can be an issue for automating the medical devices due to increased reliance on board sensors and communication mechanisms. Reducing the cost and complexity of manufacturing allows for greater accessibility to these medical devices. Reducing the cost allows for more reasonable disposability of the medical device after procedures. Reducing the complexity of the manufacturing further reduces the costs but also makes the medical device easier and faster to assemble. These design constraints together, as well as other medical device design requirements, provide a multi-faceted challenge.

Thus, a need exists for improved medical devices, including improved automation that allows for simplified user manual control, methods for detecting when the manual control has been engaged, and reduced cost and complexity.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In accordance with some embodiments, a surgical system includes an actuator system and a control system operatively coupled to the actuator system, the actuator system being operable to drive a first movable operating component and a second movable operating component. The control system includes a memory and instructions stored in the memory. The instructions cause the control system to perform actions including commanding the actuator system to operate in a first control mode, receiving a first signal containing a first kinematic parameter value of the first movable operating component, receiving a second signal containing a second kinematic parameter value of the second movable operating component, and determining a compared value derived from both the first kinematic parameter value and the second kinematic parameter value. On a condition in which the compared value is within a defined value range, the control system causes the actuator system to operate in a second control mode.

In some embodiments, the actions also include causing the control system to continue to operate the actuator system in the first control mode on a condition in which the compared value is outside the defined value range. The action that includes determining the compared value derived from both the first kinematic parameter value and the second kinematic parameter value includes combining the first kinematic parameter value and the second kinematic parameter value. The action that includes determining the compared value derived from both the first kinematic parameter value and the second kinematic parameter value includes determining a difference between the first kinematic parameter value and the second kinematic parameter value. In some embodiments, the compared value is a first compared value derived from both the first kinematic parameter value and the second kinematic parameter value according to a first calculation. The defined value range is a first defined value range. The actions can also include determining a second compared value derived from both the first kinematic parameter value and the second kinematic parameter value according to a second calculation. The actions can also include causing the control system to operate the actuator system in the instrument release mode on a condition in which the first compared value is within the first defined value range or the second compared value is within a second defined value range. In some embodiments, the first control mode can be a locked control mode. In some embodiments, the first control mode can be a teleoperated input following control mode. In some embodiments, the second control mode is an instrument release mode.

In some embodiments, the first calculation includes combining the first kinematic parameter value and the second kinematic parameter value. The second calculation includes determining a difference between the first kinematic parameter value and the second kinematic parameter value. The first defined value range is defined between zero and a first threshold value. The second defined value range is defined between zero and a second threshold value. The first kinematic parameter value corresponds to a first kinematic parameter. The first kinematic parameter is one of a torque of the first movable operating component, a speed of the first movable operating component, or a position of the first movable operating component. The second kinematic parameter value corresponds to a second kinematic parameter. The second kinematic parameter being one of a torque of the second movable operating component, a speed of the second movable operating component, or a position of the second movable operating component. The first kinematic parameter value is a moving average value of the first signal over time. The second kinematic parameter value is a moving average value of the second signal over time. The first kinematic parameter value is associated with a position of the first movable operating component. The second kinematic parameter value is associated with a position of the second movable operating component.

In some embodiments, the actions also include latching the signals to determine a baseline relative to the first kinematic parameter value and the second kinematic parameter value prior to determining the compared value. The actions can also include resetting the latched first kinematic parameter value on the condition the compared value is within the defined value range. The actions can also include resetting the latched second kinematic parameter value on the condition that the compared value is within the defined value range. The actions can also include resetting the latched first kinematic parameter value and the second kinematic parameter value on the condition the compared value is within the defined value range. In some embodiments, the actuator system includes a first rotary actuator output and a second rotary actuator output. The first rotary actuator output is positioned to drive the first movable operating component. The second rotary actuator output is positioned to drive the second movable operating component.

In some embodiments, the surgical system includes a surgical instrument. The surgical instrument includes the first movable operating component, the second movable operating component, and a manual control input. The manual control input is operably coupled to the first movable operating component and to the second movable operating component. The first movable operating component is a first tool drive member of the surgical instrument. The second movable operating component is a second tool drive member of the surgical instrument. Movement of the manual control input simultaneously moves the first movable operating component and the second movable operating component.

In some embodiments, the instrument further includes a first tool member and a second tool member. The first movable operating component is coupled to move the first tool member. The second movable operating component is coupled to move the second tool member. Movement of the manual control input causes the first tool member and the second tool member to move apart from one another. Movement of the manual control input causes the compared value to be within the defined value range. Movement of one or both of the first tool member and the second tool member by a force originating outside the surgical system causes the compared value to be outside of the defined value range. The actuator system includes a first actuator powertrain and a second actuator powertrain. The first actuator powertrain is operable to drive the first movable operating component of the instrument. The second actuator powertrain is operable to drive the second movable operating component of the instrument.

In some embodiments, commanding the actuator system to operate in the instrument release mode includes driving the first actuator powertrain to cause a first dampening torque in the first movable operating component and driving the second actuator powertrain to cause a second dampening torque in the second movable operating component. The first dampening torque is less than a torque applied to the first movable component by movement of the manual control input. The second dampening torque is less than a torque applied to the second movable component by movement of the manual control input.

In some embodiments, the actions include determining if an instrument operatively coupled to the actuator system is one of a first instrument type or a second instrument type different from the first instrument type. The actions can include commanding the actuator system to operate in the instrument release mode only on a condition in which the instrument type is the first instrument type and on the condition in which the compared value is within a defined value range. The defined value range is defined to be a first instrument defined value range for a first instrument type. The defined value range is defined to be a second instrument defined value range for a second instrument type different from the first instrument type. The second instrument defined value range is different from the first instrument defined value range.

In some embodiments, the actuator system includes a first actuator powertrain and a second actuator powertrain. The first actuator powertrain is operable to drive the first movable operating component of the instrument. The second actuator powertrain is operable to drive the second movable operating component of the instrument. The first kinematic parameter value is associated with back-drive of the first movable operating component. The second kinematic parameter value is associated with back-drive of the second movable operating component.

In accordance with other embodiments, a surgical system includes an actuator system and a control system operatively coupled to the actuator system. The actuator system is operable to drive a movable operating component. The control system includes a memory and instructions stored in the memory. The instructions cause the control system to perform actions including commanding the actuator system to operate in a teleoperated input following control mode. The instructions include receiving one or more kinematic parameter values of the movable operating component. The instructions include causing the control system to continue to operate the actuator system in the first control mode on a first condition in which the one or more kinematic parameter values of the movable operating component is outside a defined range. The instructions include causing the control system to operate the actuator system in an instrument release mode on a second condition in which the one or more kinematic parameter values of the movable operating component is within the defined range. In some embodiments, the first control mode can be a locked control mode. In some embodiments, the first control mode can be a teleoperated input following control mode. In some embodiments, the second control mode is an instrument release mode.

In some embodiments, the surgical system includes a sensor and a manual control input. The one or more kinematic parameter values of the movable operating component are based on information from the sensor and is associated with a state change of the manual control input. The manual control input is operable to drive the movable operating component. The sensor is a switch that is actuated in response to movement of the manual control input.

In some embodiments, the one or more kinematic parameter values is part of a plurality of kinematic parameter values of the movable operating component. The actions include receiving the plurality of kinematic parameter values. The actions include determining if the plurality of kinematic parameter values are associated with surgical instrument end effector movement driven by a manual input device. The defined one or more kinematic parameter is a repeating pattern. The plurality of kinematic parameter values correspond to the repeating pattern. The surgical system includes a manual control input. The manual control input is operable to drive the movable operating component. In some embodiments, the one or more kinematic parameter values are associated with a kinematic state change of the manual control input. In some embodiments, The one or more kinematic parameter values are associated with a kinematic state change of the movable operating component driven by the manual control input. In some embodiments, the one or more kinematic parameter values are associated with position, orientation, torque, speed, linear velocity, or angular velocity of the movable operating component driven by the manual control input. The one or more kinematic parameter values include a repeating pattern associated with the position, orientation, torque, speed, linear velocity, or angular velocity of the movable operating component.

In some embodiments, the movable operating component is a first movable operating component. The surgical system includes a second movable operating component. The one or more kinematic parameter values includes a first plurality of kinematic parameter values of the first movable operating component and a second plurality of kinematic parameter values of the second movable operating component. The first plurality of kinematic parameter values and the second plurality of kinematic parameter values are associated with a kinematic state change of a manual control input operable to drive the first movable operating component and the second movable operating component. In some embodiments, the first condition includes the first plurality of kinematic parameter values or the second plurality of kinematic parameter values being outside the defined range. The second condition includes the first plurality of kinematic parameter values and the second plurality of kinematic parameter values being inside the defined range value. The first condition includes the first plurality of kinematic parameter values and the second plurality of kinematic parameter values being outside the defined range. The second condition includes the first plurality of kinematic parameter values or the second plurality of kinematic parameter values being inside the defined range value.

In some embodiments the actions include determining the defined range by comparing the first plurality of kinematic parameter values and the second plurality of kinematic parameter values. The comparing the first plurality of kinematic parameter values and the second plurality of kinematic parameter values to determine the defined range includes performing a calculation. The calculation includes taking an average between the first plurality of kinematic parameter values and the second plurality of kinematic parameter values with each of the first plurality of kinematic parameter values corresponding with each of the second plurality of kinematic parameter as a function of time. The calculation includes taking a difference between the first plurality of kinematic parameter values and the second plurality of kinematic parameter values with each of the first plurality of kinematic parameter values corresponding with each of the second plurality of kinematic parameter as a function of time.

In some embodiments, movement of the manual control input simultaneously moves the first movable operating component and the second movable operating component. The surgical system includes a surgical instrument that includes the first movable operating component, the second movable operating component, and the manual control input. The surgical instrument includes a first tool member and a second tool member. The first movable operating component moves the first tool member. The second movable operating component moves the second tool member. Simultaneous movement of the first movable operating component and the second movable operating component due to rotation of the manual control input causes the first tool member and the second tool member to move apart from one another. Movement of the manual control input causes the first plurality of kinematic parameter values to be within the defined range of compared values. Movement of a first tool member or a second tool member at the distal end causes the first plurality of kinematic parameter values to be outside of the defined range of compared values.

In some embodiments, the actuator system includes a first actuator powertrain and a second actuator powertrain. The first actuator powertrain is operable to drive the first movable operating component of the instrument. The second actuator powertrain is operable to drive the second movable operating component of the instrument. The first kinematic parameter is associated with back-drive of the first movable operating component. The second kinematic parameter is associated with back-drive of the second movable operating component.

In accordance with other embodiments, a surgical system includes an actuator system and a control system operatively coupled to the actuator system. A first set of one or more values is associated with a kinematic parameter of a first movable operating component of a surgical instrument operatively coupled to the actuator system. A first condition is defined in which the first set of one or more values is not equal to a first defined value or is outside a first defined range of values. A second condition is defined in which the first set of one or more values is equal to the first defined value or is within the first defined range of values. The control system includes a memory system and instructions stored in the memory system. The instructions cause the control system to perform actions including operating the actuator system in a teleoperated input following control mode. The instructions include continuing to operate the actuator system in the first control mode during the first condition. The instructions include commanding the actuator system to stop operating in the first control mode and to operate in an instrument release mode on the second condition.

In some embodiments, the first movable operating component of the surgical instrument is operatively coupled to a manual control input and is operably coupled to move one or more end effector tool members of the surgical instrument. The first set of one or more values is based on a kinematic state change of the manual control input. The kinematic state of the manual control input is a change in position or orientation of the manual control input. The first movable operating component of the surgical instrument is a component of an end effector tool member drivetrain of the surgical instrument. The first set of one or more values is based on a kinematic state change associated with the first movable operating component The kinematic state change of the first movable operating component is associated with movement of a manual control input operable to drive the first movable operating component. The first set of one or more values is associated with a change in position, orientation, linear speed, rotational speed, linear velocity, rotational velocity of, or a change in force or torque associated with, the first movable operating component. The first set of one or more values includes a repeating pattern associated with a change in position, orientation, linear speed, rotational speed, linear velocity, rotational velocity of, or a change in force or torque associated with, the first movable operating component.

In some embodiments, a second set of one or more values is associated with a kinematic parameter of a second movable operating component of the surgical instrument. A third condition is defined in which the first set of one or more values is not equal to the first defined value or is outside the first defined range of values, or the second set of one or more values is not equal to the second defined value or is outside the second defined range of values. A fourth condition is defined in which both the set of one or more values is equal to the defined value or is within the defined range of values, and the second set of one or more values is equal to the second defined value or is within the second defined range of values. The actions include continuing to operate the actuator system in the first control mode during the third condition. The actions include commanding the actuator system to stop operating in the first control mode and to operate in the instrument release mode on the fourth condition.

In some embodiments, the first movable operating component of the surgical instrument is a component of a first end effector tool member drivetrain of the surgical instrument. The second movable operating component of the surgical instrument is a component of a second end effector tool member drivetrain of the surgical instrument. The first set of one or more values is based on a kinematic state change of the first movable operating component. The second set of one or more values is based on a kinematic state change of the second movable operating component. The kinematic state changes of the first movable operating component and the second movable operating component are associated with movement of a manual control input of the surgical instrument operable to drive the first movable operating component and a second movable drive component.

In some embodiments, the surgical system includes the surgical instrument. The surgical instrument includes the manual control input, a first end effector tool member driven by the first end effector tool member drive train, and a second end effector tool member driven by the second end effector tool member. Movement of the manual control input drives the first end effector tool member and the second end effector tool member away from each other. A first individual kinematic parameter value is associated with the first movable component and a second individual kinematic parameter value is associated with the second movable component. The actions include determining a first compared value derived from both the first individual kinematic parameter value and the second individual kinematic parameter value.

In some embodiments, the actions include determining a second compared value derived from both the individual kinematic parameter value and the second individual kinematic parameter value. The first set of one or more values includes the first compared value. The first defined range of values is a first defined range of compared values. The second set of one or more values includes the second compared value. The second defined range of values is a second defined range of compared values. The third condition is one in which the first compared value is outside the first defined range of compared values. The fourth condition is one in which the second compared value is within the second defined range of compared values. The first compared value is represents correspondence between the first individual kinematic parameter value and the second individual kinematic parameter value. The second compared value represents a difference between the first individual kinematic parameter value and the second individual kinematic parameter value. The first compared value is derived from the individual kinematic parameter value and the second individual kinematic parameter value is an average value of the first individual kinematic parameter value and the second individual kinematic parameter value.

In some embodiments, the surgical system includes the surgical instrument. The surgical system includes the surgical instrument. The surgical instrument includes a manual control input operably coupled to the first movable component. Movement of the manual control input drives the first movable component. The surgical system includes the surgical instrument. The surgical instrument includes a manual control input operably coupled to the first movable component and the second movable component. Movement of the manual control input drives the first movable component and the second movable component. The first set of one or more values is associated with back-drive of the first movable operating component. The second set of one or more values is associated with back-drive of the second movable operating component. The first set of one or more values is associated with back-drive of the first movable operating component.

In accordance with other embodiments, a method of control for a surgical system includes providing a controller operatively coupled to an actuator system. The actuator system is operable to drive a first movable operating component and a second movable operating component. The method including commanding the actuator system to operate in a first control mode. The method including receiving a first signal containing a first kinematic parameter value of the first movable operating component. The method includes receiving a second signal containing a second kinematic parameter value of the second movable operating component. The method includes determining a compared value derived from both the first kinematic parameter value and the second kinematic parameter value. On a condition in which the compared value is within a defined value range, the method includes causing the control system to operate the actuator system in an instrument release mode.

In accordance with other embodiments, a method of control for a surgical system includes providing the surgical system. The surgical system includes a controller operatively coupled to an actuator system. The actuator system is operable to drive a movable operating component. A first set of one or more values is associated with a kinematic parameter of a first movable operating component. The movable operating component is operatively coupled to the actuator system. The method includes operating the actuator system in a first control mode. The method includes continuing to operate the actuator system in the first control mode during the first condition in which the first set of one or more values is not equal to a first defined value or is outside a first defined range of values. The method includes commanding the actuator system to stop operating in the first control mode and to operate in an instrument release mode on the second condition in which the first set of one or more values is equal to the first defined value or is within the first defined range of values.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5 is a schematic illustration of a controller for use with a minimally invasive teleoperated surgery system according to an embodiment.

FIG. 6A is a diagrammatic illustration of a portion of a medical device for manual tool adjustment with an antagonistic drive according to an embodiment.

FIG. 6B is a diagrammatic illustration of a portion of a medical device for manual tool adjustment with a self-antagonistic drive according to an embodiment.

FIG. 7 is a flow chart for control of the surgical system of FIGS. 6A and 6B in response to using a manual user input according to an embodiment.

FIG. 8A is a graph of a first signal and a second signal received from a first drive motor and a second drive motor, respectively, in the surgical system of FIG. 6A according to an embodiment, FIG. 8B is a graph of the common mode of the signals in FIG. 8A relative to a first range, and FIG. 8C is a graph of the differential mode of the signals of 8A relative to a second range.

FIG. 8D is a graph of a first signal and a second signal received from a first drive motor and a second drive motor, respectively, in the surgical system of FIG. 6A according to an embodiment. FIG. 8E is a graph of the common mode of the signals in FIG. 8D relative to a first range, FIG. 8F is a graph of the differential mode of the signals of 8D relative to a second range.

FIG. 8G is a graph of a first signal and a second signal received from a first drive motor and a second drive motor, respectively, in the surgical system of FIG. 6A according to an embodiment, FIG. 8H is a graph of the common mode of the signals in FIG. 8D relative to a first range, and FIG. 81 is a graph of the differential mode of the signals of 8A relative to a second range.

FIG. 9 is a diagrammatic illustration of a portion of a medical device for manual tool adjustment according to an embodiment.

FIG. 10 is a flow chart for control of the surgical system of FIG. 9 in response to using a manual user input according to an embodiment.

FIG. 11 is a representative graph of a signal received from a first sensor in a surgical system representative of the surgical system of FIG. 9 compared to a graph of a defined signal based on the use of a manual input according to an embodiment.

FIG. 12 is a diagrammatic illustration of a portion of a medical device for manual tool adjustment according to an embodiment.

FIG. 13 is a flow chart for control of the surgical system of FIG. 12 in response to using a manual user input according to an embodiment.

FIG. 14 is a perspective view of a portion of a medical device for manual tool adjustment according to an embodiment.

FIG. 15 is a perspective view of a mechanical structure of the medical device of FIG. 14.

FIG. 16 is an exploded view of a portion of the medical device of FIG. 14.

FIG. 17 is a top perspective view of cable routing of the medical device of FIG. 14.

FIG. 18 is a perspective view of a tool drive structure of the medical device of FIG. 14.

FIGS. 19A and 19B are views of a capstan of the medical device of FIG. 14, with FIG. 19A being a side view in a first state and FIG. 19B being a side view in a second state.

FIGS. 20A and 20B are views of a drive disc of the medical device of FIG. 14, with FIG. 20A being a side view and FIG. 20B being a perspective view.

FIGS. 21A-C are views of a manual drive input member of the manual drive mechanism of FIG. 14, with FIG. 21A being a side view, FIG. 21B being a top view, and FIG. 21C being a perspective view.

FIGS. 22A-C are views of a manual drive coupling member of the manual drive mechanism of FIG. 14, with FIG. 22A being a side view, FIG. 22B being a top view in a first state, and FIG. 22C being a top view in a second state.

FIGS. 23A-C are views of a support bracket, with FIG. 23A being a top view, FIG. 23B being a bottom view, and FIG. 23C being a perspective view.

FIGS. 24A and 24B are perspective views of a manual drive mechanism of the medical device of FIGS. 14-15 with components removed for simplicity, with FIG. 24A in a first state and FIG. 24B in a second state.

FIGS. 25A and 25B are bottom views of portions of the manual drive mechanism of the medical device of FIGS. 14-15 with components removed for simplicity, with FIG. 24A in a first state and FIG. 24B in a second state.

FIG. 26 is a flow chart schematic illustrating control and feedback forces within the surgical system of FIG. 14 according to an embodiment.

FIG. 27 is a flow chart for control of the surgical system of FIG. 14 in response to using a manual user input according to an embodiment.

FIG. 28A is a graph of a first signal and a second signal received from a first drive motor and a second drive motor, respectively, in the surgical system of FIG. 14 in response to the use of a manual input in accordance with one operational example, FIG. 28B is a graph of the common mode of the signals in FIG. 28A, and FIG. 28C is a graph of the differential mode of the signals of 28A.

FIG. 28D is a graph of a first signal and a second signal received from a first drive motor and a second drive motor, respectively, in the surgical system of FIG. 14 in response to an end effector disturbance in accordance with one operational example, FIG. 28E is a graph of the common mode of the signals in FIG. 28D, and FIG. 28F is a graph of the differential mode of the signals of 28D.

FIG. 29A is a perspective view of a portion of a medical device for manual tool adjustment according to an embodiment and FIG. 29B is a perspective view of a manual input from the medical device of FIG. 29A.

FIG. 30 is a perspective view of a portion of a medical device for manual tool adjustment according to an embodiment.

### DETAILED DESCRIPTION

The embodiments described herein can advantageously be used in a wide variety of grasping, cutting, and manipulating operations associated with minimally invasive surgery. In some embodiments, an end effector of the medical device can move with reference to the main body of the instrument in three mechanical degrees of freedom (DOFs), e.g., pitch, yaw, and roll (shaft roll). There may also be one or more mechanical DOFs in the end effector itself, e.g., two jaws, each rotating with reference to a clevis (2 DOFs) and a distal clevis that rotates with reference to a proximal clevis (one DOF).

The medical devices of the present application enable motion in three degrees of freedom (e.g., about a pitch axis, a yaw axis, and a grip axis) using multiple cables. In some embodiments, four cables are used, thereby reducing the total number of cables required, reducing the space required within the shaft and wrist, reducing overall cost, and enabling further miniaturization of the wrist and shaft assemblies to promote MIS procedures. In some embodiments, six cables are used. It is appreciated that the various embodiments provided herein are adaptable to other systems with more or fewer cables based on the disclosure provided herein.

Moreover, the instruments described herein include a manual drive mechanism that provide a manual input into the medical device such that the end effector and tools thereof can be actuated via the manual input. Furthermore, the medical device automatically alternates operation modes such as alternating from an active input following mode, to an instrument locked mode, to an instrument release mode in response to use of the manual input. Other modes are contemplated as well. Automatic switching between modes removes the burden and the knowledge requirement for having the surgeon trigger the different modes from the user input control device and makes it easier to release a jammed tool during surgery. Having the manual input device engage and the medical device change modes automatically when desired removes undesirable distractions and reduces the chances of incorrect operation of the medical device.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end (i.e., the end manipulated by the user or coupled to the actuation shaft) would be the proximal end of the medical device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements such as location) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial positions and orientations. The combination of a body's position and orientation defines the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc., but do not preclude the presence or addition of one or more other features, steps, operations, elements, components, or groups.

As used in this specification and the appended claims, the word “member” refers to a constituent portion of a larger structure or mechanism. A “member” can refer to an individual contiguous structure or multiple connected structures such as a mechanism.

Unless indicated otherwise, the terms apparatus, medical device, medical instrument, and variants thereof can be interchangeably used.

Aspects of the invention are described primarily in terms of an implementation using a da Vinci® surgical system, commercialized by Intuitive Surgical, Inc. of Sunnyvale, California. Examples of such surgical systems are the da Vinci Xi® surgical system (Model IS4000), da Vinci X® Surgical System (Model IS4200), and the da Vinci Si® surgical system (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® surgical systems (e.g., the Model IS4000, the Model IS3000, the Model IS2000, the Model IS1200, the Model SP1099) are merely presented as examples, and they are not to be considered as limiting the scope of the inventive aspects disclosed herein. As applicable, inventive aspects may be embodied and implemented in both relatively smaller, hand-held, hand-operated devices that are not mechanically grounded in a world reference frame and relatively larger systems that have additional mechanical support that is grounded in a world reference frame.

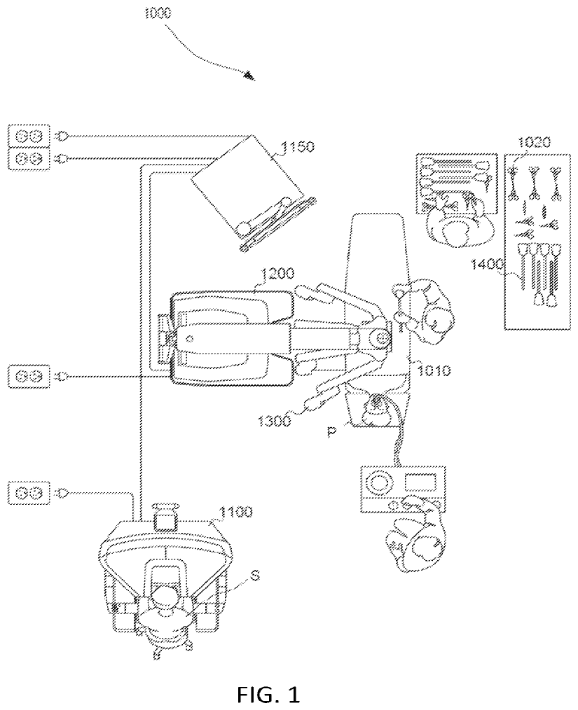

FIG. 1 is a plan view illustration of a teleoperated surgical system 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by a surgeon or other skilled clinician S during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

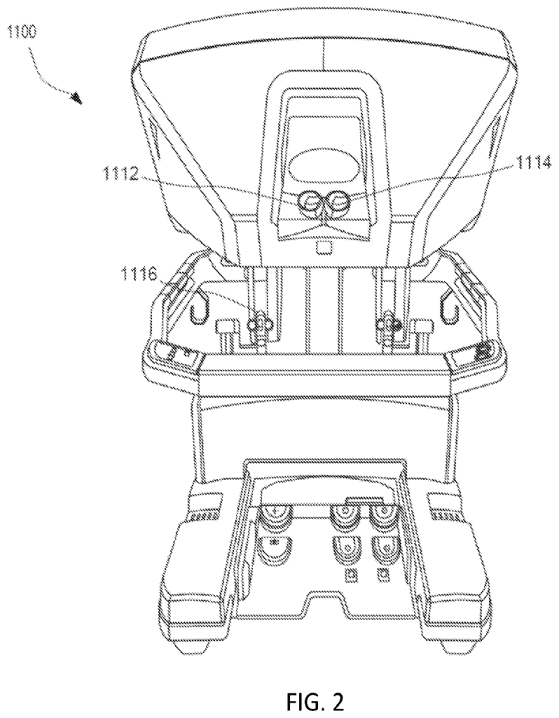

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

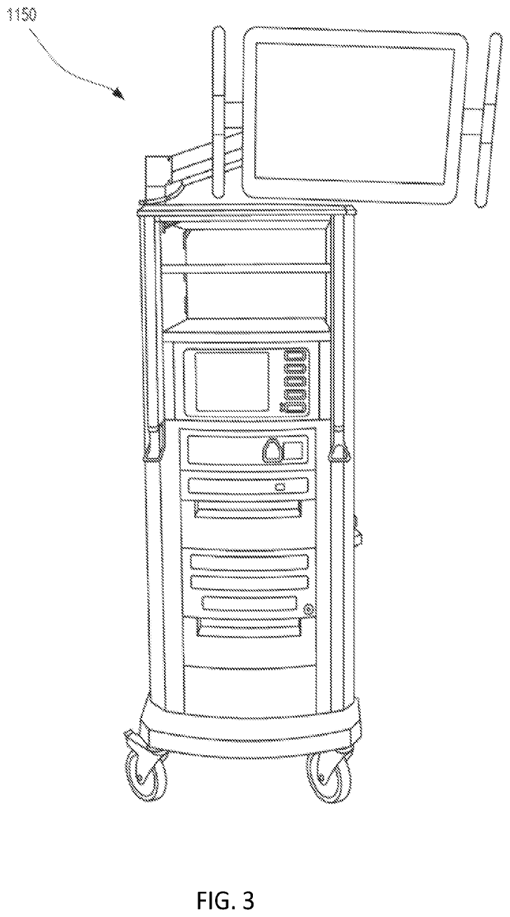

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

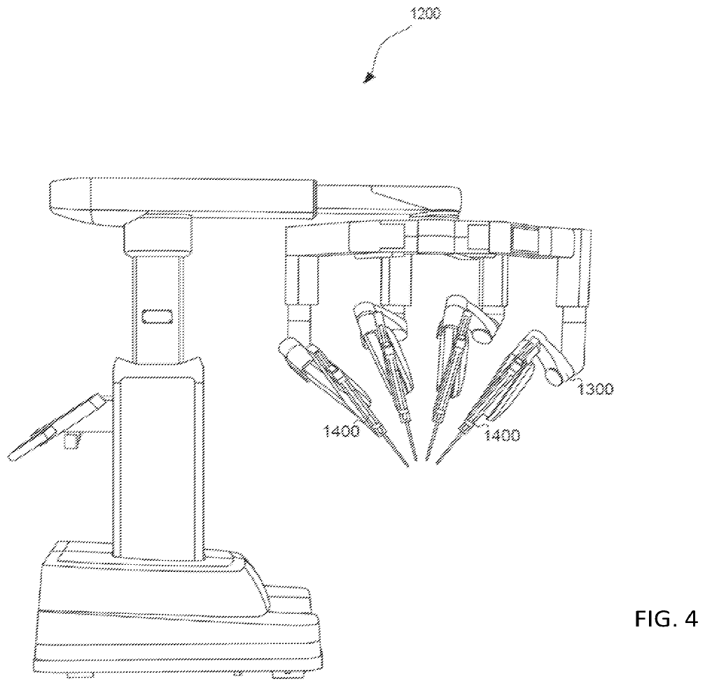

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

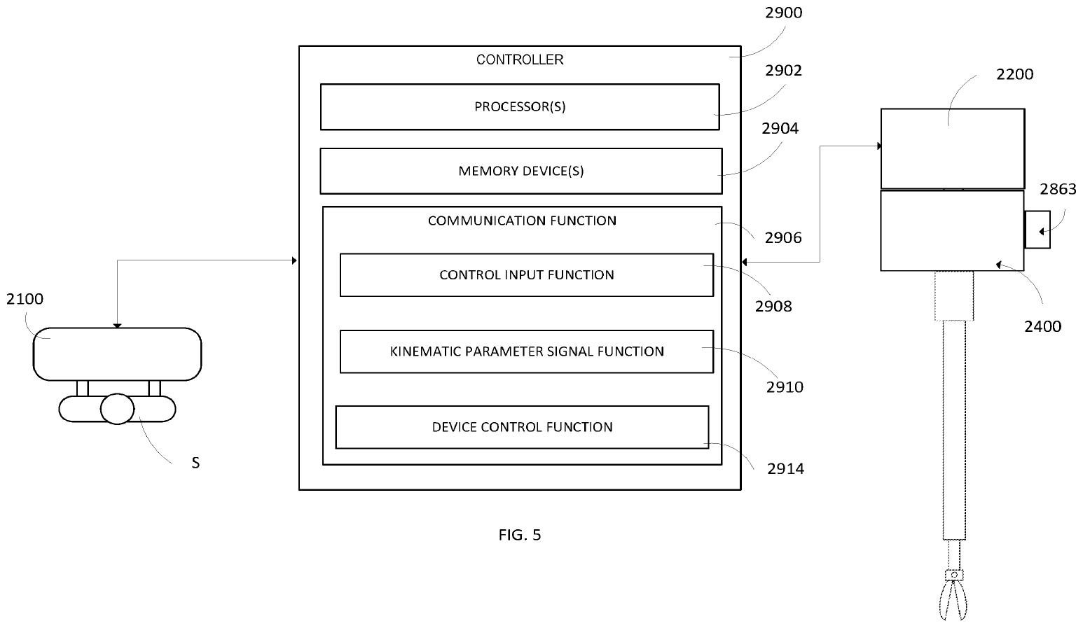

FIG. 5 is a schematic illustration of a surgical controller 2900 in communication with a control unit 2100, a manipulator unit 2200, and an instrument 2400. The surgical controller 2900 implements the methods, processes, and systems for the utilization of the control unit 2100, the manipulator unit 2200, and the instrument 2400 along with the features, functions, and operations thereof. The surgical controller 2900 can be included in any of the systems and can implement methods associated with any of the instruments described herein, such as the methods and processes discussed in more detail below such as 3010, 4010, 5010, 8002, and 8050. Similar to the systems discussed above with respect to MIRS 1000, the manipulator unit 2200 can manipulate at least one removably coupled instrument 2400 through a minimally invasive incision in the body or natural orifice of the patient while the surgeon S views the surgical site and controls movement of the instrument 2400 through control unit 2100 with the assistance of the surgical controller 2900. In addition to and as a part of producing signals and commands associated with the movement of the instrument 2400, the surgical controller 2900 detects current operating conditions of the medical instrument 2400. One example of such monitoring of the operating conditions includes the monitoring of rejection and detection conditions for instrument (e.g., 3400, 4400, 5400, 6400, or 7400). More specifically, the rejection and detection conditions relate to instrument interferences during surgical procedures such as the jamming of an end effector on tissue. Another example of such operating conditions includes the monitoring of the use of a manual input device 2863 on instrument 2400. In response to the detection of certain conditions, the surgical controller 2900 also automatically changes system functionality modes for the surgical system (e.g., 1000). For example, under certain conditions, the surgical controller 2900 allows or causes the system to function under a teleoperated input following control mode. In another example, under certain conditions (e.g., when the end effector movement has unexpectedly been restricted (e.g., has been jammed or caught on tissue), the surgical controller 2900 allows or causes the system to enter into an instrument locked mode. In another example, under certain conditions (e.g., when the manual input device 2863 has been actuated), the surgical controller 2900 allows or causes the system to function under an instrument release mode. These modes are discussed in more detail below.

As depicted, the controller 2900 includes one or more processor(s) 2902 and associated memory device(s) 2904 configured to perform a variety of computer-implemented functions (e.g., performing the methods, steps, calculations and the like and storing relevant data as disclosed herein). Additionally, in some embodiments, the controller 2900 includes a communication module 2906 to facilitate communications between the controller 2900 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 2904 may generally comprise memory element(s) including, but not limited to, computer readable medium (e.g., random access memory (RAM)), computer readable nonvolatile medium (e.g., a flash memory), a floppy disk, a compact disc read only memory (CD ROM), a magneto optical disk (MOD), a digital versatile disc (DVD) and/or other suitable memory elements. Such memory device(s) 2904 may generally be configured to store suitable computer readable instructions that, when implemented by the processor(s) 2902, configure the controller 2900 to perform various functions.

The communication function 2906 may include a control input function 2908, a kinematic parameter signal function 2910, and a device control function 2914. The control input function 2908 is configured to receive control inputs from the operator/surgeon S, such as via the input device of the user control unit 2100. The device control function 2914 is configured to send input signals to the manipulator unit 2200 or instrument 2400. These input signals are used to drive the operational functionality of the manipulator unit 2200 which can in turn drive the operational functionality of the instrument 2400. The controller 2900 can process signals from the control input function 2908 and forward operational signals to the manipulator unit 2200 or instrument 2400 for control of the instrument 2400 during operation.

As used herein, a kinematic parameter includes, as examples, position (e.g., location, orientation, or other special constraint), speed, acceleration, torque, or any other characteristic that reflects information about the changing configuration of movable mechanical components. A kinematic parameter value includes information that can be contained within a signal the reflects the kinematic parameters of movable components within the system.

The kinematic parameter signal function 2910 is configured to receive information from the manipulator unit 2200 or the instrument 2400. In some embodiments, the manipulator unit 2200 can include electromechanical actuators. The kinematic parameter signal function 2910 can receive signals from one or more of these electromechanical actuators (e.g., a position of the electromechanical actuator as a function of time). Processing these signals with the controller 2900 yields information related to the instrument 2400, such as whether or not a manual input device 2863 is being used on the instrument. Accordingly, the kinematic parameter signal function 2910 is configured to receive a signal such that the controller 2900 can process that signal to indicate use of the manual input device 2863. Upon a determination that the manual input device 2863 is in use, the device control function can send control signals to the manipulator unit 2200 or instrument 2400 to control of the instrument 2400 as appropriate under the observed conditions. For example, if the manual input device 2863 is not being used, the controller allows continued use of the system or allows the system to remain in other modes including for example, instrument locked mode or a teleoperated input following control mode. If a determination is made that the manual input device 2863 is in use, the controller switches modes to an instrument release mode, allowing for the user to operate the manual input device 2863.

While the controller 2900 is discussed above with reference to manipulator unit 2200 and instrument 2400 merely as an example, it is appreciated that this disclosure is also applicable to other manipulators and instruments as discussed herein, e g., instrument 3400, instrument 4400, instrument 5400, instrument 6400, or instrument 7400.

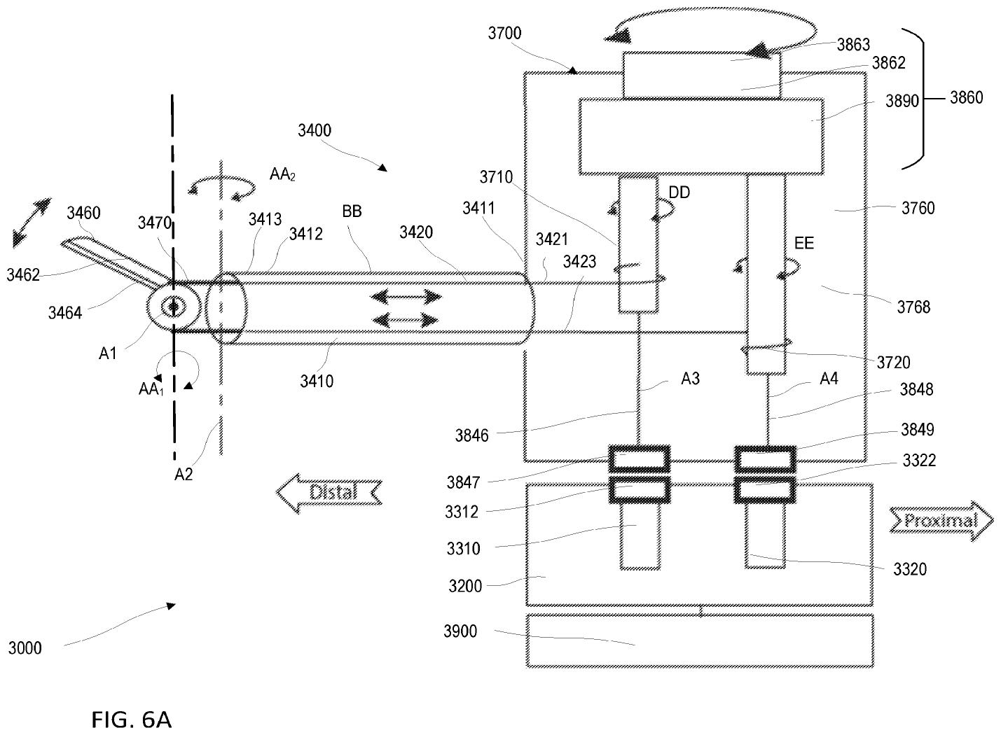

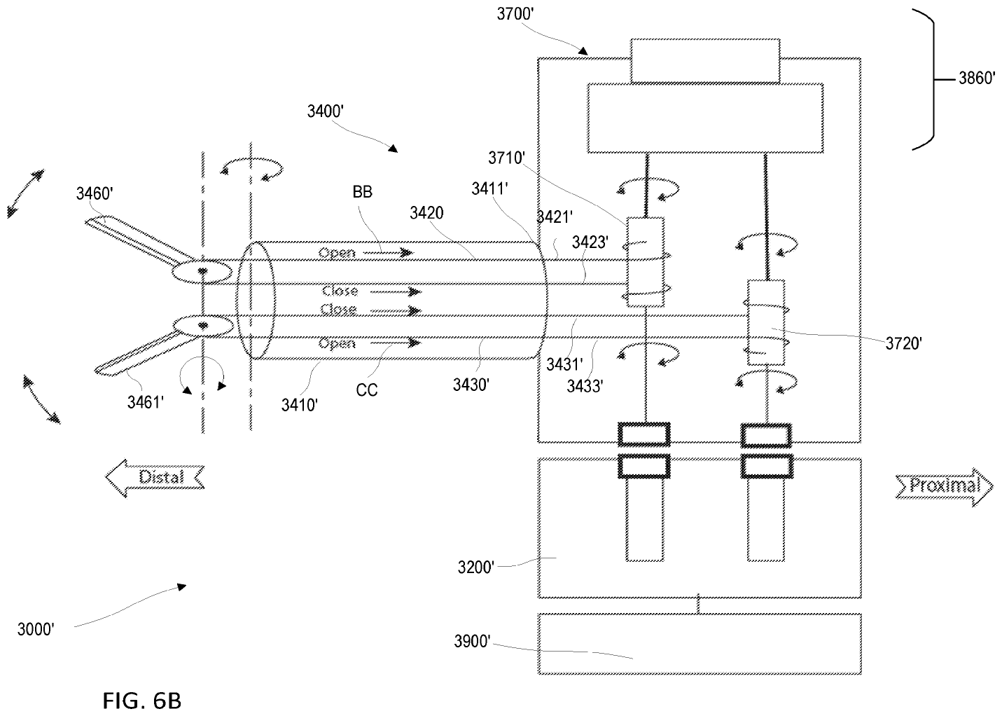

FIG. 6A is a schematic illustration of a portion of a surgical system 3000 according to an embodiment. The surgical system 3000 includes a surgical instrument 3400, a manipulator unit 3200, and a controller 3900. The manipulator unit 3200 (which functions as an actuator system) is in communication with the controller 3900 such that the controller 3900 can send and receive signals from the manipulator unit 3200. For example, the controller 3900 can receive input control signals from the input control unit (such as user control unit 1100 in FIG. 1 or user control unit 2100 in FIG. 5) based on surgeon input (receivable for example at the control input function 2908 of controller 2900 in FIG. 5). The controller 3900 then sends control signals to the manipulator unit 3200 (as an example, the device control function 2914 of controller 2900 sends the control signals as illustrated in FIG. 5) which, in turn, drives the surgical instrument 3400. In this manner the manipulator unit 3200 (and any other manipulator units described herein) functions as an actuator system that actuates the surgical instrument 3400. The surgical instrument includes a mechanical structure 3700 having a manual drive mechanism 3860. As discussed in more detail below the manual drive mechanism 3860 drives a powertrain that includes a first tool drive member 3710, input member 3846, and input disk 3847 and a powertrain that includes a second tool drive member 3720, input member 3848, and input disk 3849. These powertrain components are also variously referred to as movable components. As shown in FIG. 6A, the manual drive mechanism 3860 includes mechanical components to drive the two powertrains together based on a single actuation of the manual input device 3863. The first powertrain can also be driven by electromechanical drive 3310 and the second powertrain can also be driven by electromechanical drive 3320. Drive disk 3312 can engage with input disk 3847 and drive disk 3322 can engage with input disk 3849. Due to the engagement between the disks, the manual drive mechanism 3860 is also sufficiently mechanically coupled with electromechanical drive 3310 and electromechanical drive 3320 to drive (or cause some movement of) electromechanical drive 3310 and electromechanical drive 3320. Because of the mechanical coupling between the electromechanical drives 3310 and 3320 and the respective powertrains in the instrument 3400, the electromechanical drives 3310 and 3320 can produce signals that reflect one or more kinematic parameters of the movable components (e.g., components of the powertrains) in the surgical instrument 3400. This allows the controller 3900 to recognize use of the manual drive 3863 and change operation modes to accommodate the use of the manual drive 3863.

As shown in FIG. 6A, in some embodiments, the medical device 3400 includes a shaft 3410, a tension member 3420, an end effector 3460, and the mechanical structure 3700. The mechanical structure 3700 functions to receive one or more motor or manual input forces or torques and mechanically transmit the received forces or torques to move the end effector 3460. For example, as described above the manipulator unit 3200 can include one or more electric motors (which function as an electromechanical drive, e.g., the electromechanical drive 3310) to provide an input to the mechanical structure 3700, which in turn transmits the input via the tension member 3420 to control the end effector 3460). Specifically, the mechanical structure includes a chassis 3768, a first tool drive member 3710, a second tool drive member 3720, and a manual drive mechanism 3860. The chassis 3768 provides the structural support for mounting or supporting and aligning the components of the mechanical structure 3700. For example, openings, protrusions, mounting brackets and the like can be defined in or on chassis 3768. In some embodiments, the chassis 3768 can include multiple portions, such as an upper chassis and a lower chassis. In some embodiments, a housing 3760 can optionally enclose at least a portion of the chassis 3768.

The first tool drive member 3710 is mounted to the mechanical structure 3700 (e.g., within the housing 3760) via a first tool drive member support member (not shown). For example, the first tool drive member support member can be a mount, shaft, or any other suitable support structure to secure the first tool drive member 3710 to the mechanical structure 3700. The first tool drive member 3710 includes (or is coupled to) a first input member 3846 and an input disk 3847. The first motor drive input member 3846 can be connected to and receive mechanical input from the electromechanical drive 3310. The second tool drive member 3720 is mounted to the mechanical structure 3700 (e.g., within the housing 3760) via a second tool drive member support member (not shown). For example, the second tool drive member support member can be a mount, shaft, or any other suitable support structure to secure the second tool drive member 3720 to the mechanical structure 3700. The second tool drive member 3720 includes (or is coupled to) a second input member 3848 and a drive disk 3849. The first tool drive member 3710 can be operable to be rotated about an axis A3 in a direction DD, as shown in FIG. 6A. The second tool drive member 3720 can be operable to be rotated about an axis A4. In some embodiments, the axis A4 is parallel to the axis A3.

The manual drive mechanism 3860 is connected to and drives the first tool drive member 3710) and the second tool drive member 3720. Thus, the first tool drive member 3710 can be driven by each of the manual drive mechanism 3860 and the first input member 3846. Similarly, the second tool drive member 3720 can be driven by each of the manual drive mechanism 3860 and the second input member 3848. Similarly stated, each tool drive member 3710, 3720 can be driven by an electromechanical drive (e.g., 3310, 3320) and a manual drive mechanism 3860. As discussed in more detail below, the tool drive members 3710, 3720 are connected to and manipulate an end effector 3460. Thus, the end effector 3460 can be manipulated by either a drive motor forming a part of the manipulator unit or the manual drive mechanism 3860).

The manual drive mechanism 3860 includes manual input device 3863, a manual drive input member 3862, and a manual drive coupling member 3890. The manual drive input member 3862 is mechanically connected to the manual input device 3863. The manual input device 3863 includes a portion that is exposed to the exterior of the medical device 3400. The user can engage the manual input device 3863 and manipulate the manual drive mechanism 3860, thereby manipulating the end effector 3460. The exposed portion manual input device 3863 can include any suitable structure for receiving the user's input force. For example, the manual input device 3863 can include a rotatable wheel, a rotatable knob, a push button, a slide, or other suitable mechanical structures that receive the user's input force and allows the manual drive mechanism 3860 to translate the user's input motion to an input on the tool drive members 3710, 3720 and thereby manipulate the end effector 3460.

The manual drive coupling member 3890 is connected to the manual drive input member 3862. The manual drive coupling member 3890 transmits the user's force from the manual drive input member 3862 to both the first tool drive member 3710 and the second tool drive member 3720. Because the first tool drive member 3710 and the second tool drive member 3720 can operate independently of one another (e.g., they can rotate independently, sometimes in the same direction, sometimes in opposite directions of one another, and sometimes one can be stationary while the other rotates), the manual drive coupling member 3890 also limits interference between one or more of the combinations of the first tool drive member 3710, the second tool drive member 3720, and the manual drive input member 3862. For example, the manual drive coupling member 3890 includes selectable engage-ability between the manual drive input member 3862 and one or both of the first tool drive member 3710 and the second tool drive member 3720. Such selectable engage-ability allows input forces to be transmitted between the manual drive input member 3862 and the first tool drive member 3710 and the second tool drive member 3720 in response to the manual drive coupling being in a first state but limiting or preventing forces to be transmitted between the manual drive input member 3862 and the first tool drive member 3710 and the second tool drive member 3720 in response to the manual drive coupling being in a second state.

In another example, the manual drive coupling member 3890 allows the input force to be transmitted from the manual drive input member 3862 toward the first tool drive member 3710 and the second tool drive member 3720, but not from the first tool drive member 3710 and the second tool drive member 3720 toward the manual drive input member 3862. Said another way, in some embodiments, the manual drive coupling member 3890 does not allow movement of either of the first tool drive member 3710 or the second tool drive member 3720 to cause movement of the manual drive input member 3862.

In another example, the manual drive coupling member 3890 allows input forces to be transmitted from the manual drive input member 3862 to the first tool drive member 3710 and the second tool drive member 3720. Additionally, the manual drive coupling member 3890 allows input forces to be transmitted from the first tool drive member 3710 and the second tool drive member 3720 to the manual drive input member 3862. The manual drive coupling member 3890, however, limits input forces at the first tool drive member 3710 from being transmitted to the second tool drive member 3720 through the manual drive coupling member 3890. The manual drive coupling member 3890 also limits input forces at the second tool drive member 3720) from being transmitted to the first tool drive member 3710 through the manual drive coupling member 3890.

The manual drive coupling member 3890 can include any suitable structure or components to perform the functions described herein. For example, in some embodiments, the manual drive coupling member 3890 can include a gear member or set of gears that can be engaged and disengaged from either (or both) of the first tool drive member 3710 and the second tool drive member 3720. In other embodiments clutches, tension members, hydraulics, slidable linkages, or any other suitable mechanism for transmitting force from the user to the tool drive members some of which are discussed herein and others that a person of ordinary skill in the art would apply based on the disclosure provided herein.

The tension member 3420 includes a first proximal portion 3421, a second proximal portion 3423 and a distal portion 3422. The first proximal portion 3421 and the second proximal portion 3423 are each coupled to the mechanical structure 3700, and the distal portion 3422 is coupled to the end effector 3460. The shaft 3410 includes a proximal end portion 3411 and a distal end portion 3412 and defines a passageway 3413 that extends lengthwise through the shaft between the proximal and distal end portions. In accordance with various embodiments, the tension member includes any member suitable tension member for transmitting force between the tool drive members 3710, 3720 and the end effector. For example, the tension member can include one or more of a cable, band, strap, string, wire, tube, rod, etc. The tool drive members 3710, 3720 include one or more of capstans, winches, spools, or other suitable devices for containing, controlling, taking up, and dispensing the tension member 3420.

The end effector 3460 is rotatably coupled to the distal end portion 3412 of the shaft 3410 and includes at least one tool member 3462. The medical device 3400 is configured such that movement of the first proximal portion 3421 and the second proximal portion 3423 of the tension member 3420 produces movement of the tool member 3462 about a first axis of rotation A1 (which functions as the yaw axis; the term yaw is arbitrary), in a direction of arrows AA1. In some embodiments, the medical device 3400 can include a wrist assembly including one or more links that couples the end effector 3460 to the distal end portion 3412 of the shaft 3410. In such an embodiment, movement of the first proximal portion 3421 and the second proximal portion 3423 of the tension member 3420 can also produce movement of the wrist assembly about a second axis of rotation A2 (which functions as the pitch axis) or both movement of the wrist assembly and the end effector 3460. See, for example, U.S. provisional application No. 63/233,904 entitled “Surgical Instrument Cable Control and Routing Structures” filed on Aug. 17, 2021, which is incorporated herein by reference in its entirety.

The tool member 3462 includes a contact portion 3464 and a drive pulley 3470. The contact portion 3464 is configured to engage or manipulate a target tissue during a surgical procedure. For example, in some embodiments, the contact portion 3464 can include an engagement surface that functions as a gripper, cutter, tissue manipulator, or the like. In this manner, the contact portion 3464 of the tool member 3462 can be actuated to engage or manipulate a target tissue during a surgical procedure. The tool member 3462 (or any of the tool members described herein) can be any suitable medical tool member. Moreover, although only one tool member 3462 is shown, in other embodiments, the medical device 3400 can include two or more moving tool members that cooperatively perform gripping or shearing functions.

The tension member 3420 is routed from the mechanical structure 3700 to the end effector 3460 and then back to mechanical structure 3700, and each individual end of the tension member 3420 is coupled to either the first tool drive member 3710 or the second tool drive member 3720 of the mechanical structure 3700. More specifically, the first proximal portion 3421 of the tension member 3420 is coupled to the first tool drive member and the second proximal portion 3423 is coupled to the second tool drive member 3720 of the mechanical structure 3700. In other words, the two ends of a single tension member (e.g., 3420) are coupled to and actuated by two separate tool drive members of the mechanical structure 3700.

More specifically, the two ends of the tension member 3420 that are associated with opposing directions of a single degree of freedom are connected to two independent tool drive members 3710 and 3720. This arrangement, which is generally referred to as an antagonist drive system, allows for independent control of the movement of (e.g., pulling in or paving out) each of the ends of the tension member 3420. The mechanical structure 3700 produces movement of the tension member 3420, which operates to produce the desired articulation movements (pitch, yaw, or grip) at the end effector 3460. Accordingly, as described herein, the mechanical structure 3700 includes components and controls to move the first proximal portion 3421 of the tension member 3420 via the first tool drive member 3710 in a first direction (e.g., a proximal direction) and to move the second proximal portion 3423 of the tension member 3420 via the second tool drive member 3720 in a second opposite direction (e.g., a distal direction). The mechanical structure 3700 can also move both the first proximal portion of the tension member 3420 and the second proximal portion of the tension member 3420 in the same direction. In this manner, the mechanical structure 3700 can maintain the desired tension within the tension members to produce the desired movements at the end effector 3460.

In other embodiments, any of the medical devices described herein can have the two ends of the tension member wrapped about a single tool drive member as illustrated as an example in FIG. 6B. This alternative arrangement, which is generally referred to as a self-antagonist drive system, operates the two ends of the tension member using a single drive motor. The surgical system 3000′ includes a surgical instrument 3400′, the manipulator unit 3200′, and the controller 3900′. The surgical instrument 3400′ is similar to the surgical instrument 3000 described above with reference to FIG. 6A but is a self-antagonist drive system having two ends of each tension member (e.g., 3420′ and 3410′) driven by a single drive motor. Specifically, the surgical instrument 3400′ includes two tension members 3420′ and 3430″. Both proximal ends 3421′ and 3423 of tension member 3420′ are wrapped around tool drive member 3710′. Both proximal ends 3431′ and 3433′ of tension member 3430′ are wrapped around tool drive member 3720′. Tension member 3420′ mechanically couples tool drive member 3710′ to tool 3460′. Tension member 3430′ mechanically couples tool drive member 3720′ to tool 3461′. As each of the tool drive members rotate one way one portion of the respective tension member pays out while the other portion of the tension member pulls in creating self-antagonistic drives.

In addition, in some alternative embodiments, the tension member includes two tension member segments, with each tension member segment having a distal end portion that is coupled to the end effector and a proximal end portion wrapped about a tool drive member-either separate tool drive members as in the antagonist drive arrangement or a single common tool drive member in the self-antagonist drive arrangement. Descriptions herein referring to the use of a single tension member incorporate the similar use of two separate tension member segments.

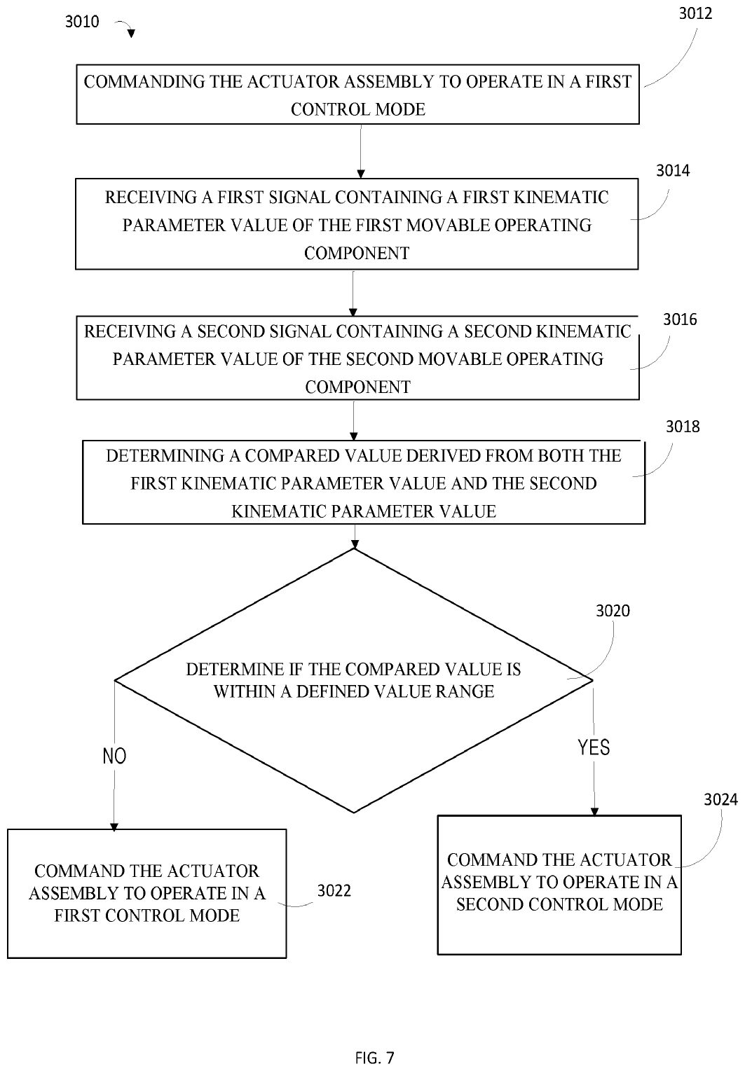

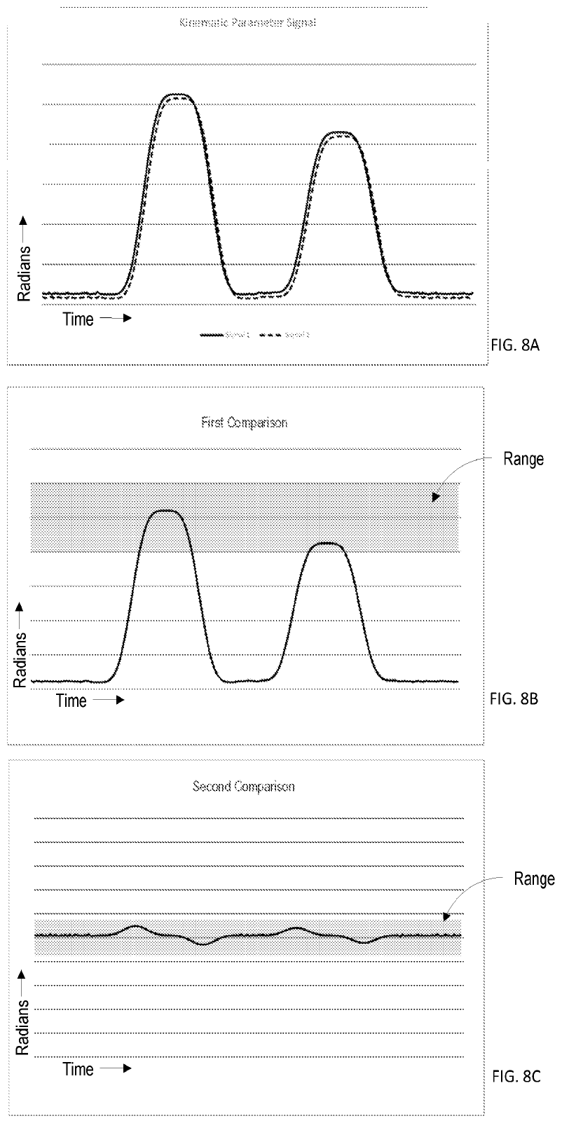

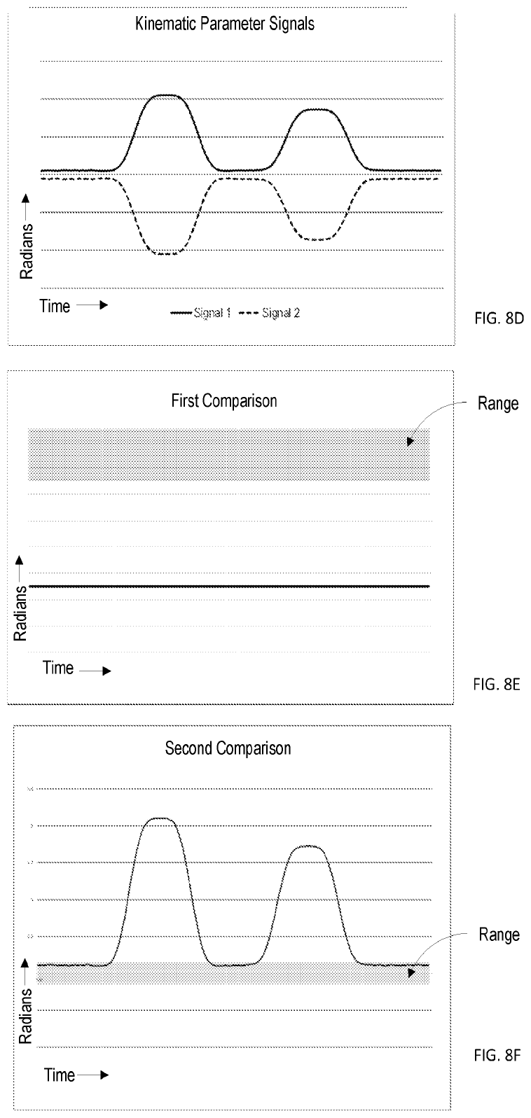

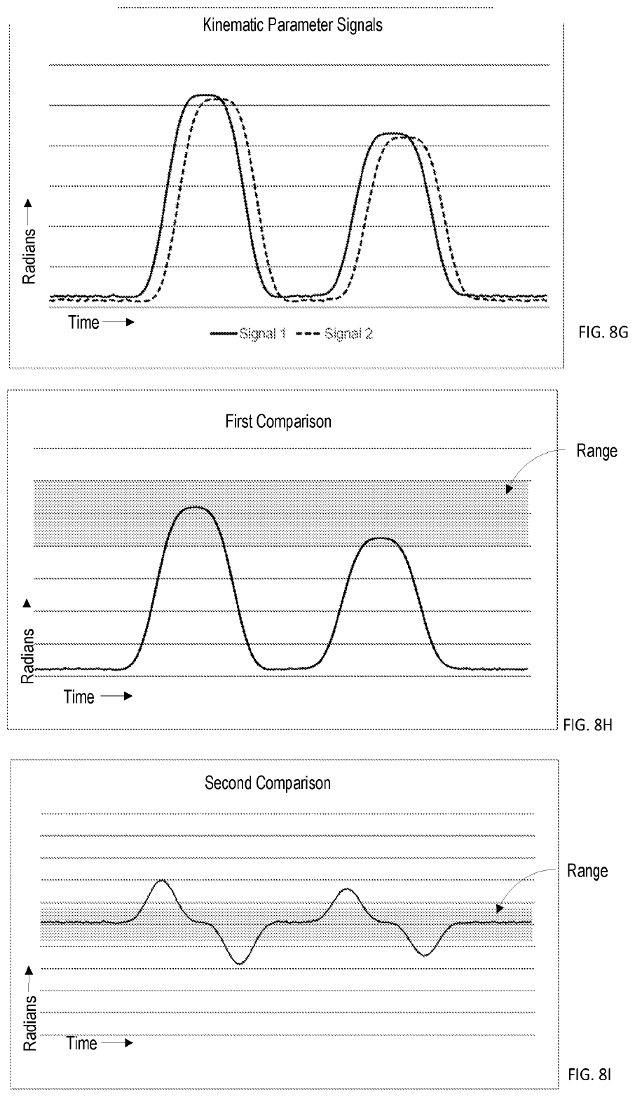

FIG. 7 is a flow chart of a control method 3010 for a surgical system according to an embodiment. The method 3010 can, in an embodiment, be performed via the surgical system as described with reference to any of the systems shown in FIGS. 1-6B, or any other systems herein. However, it should be appreciated that in various embodiments, aspects of the method 3010 may be accomplished via additional embodiments of the surgical system or components thereof, such as instrument 8400. FIGS. 8A-I show examples of signals and signal processing for determining control of the surgical system 3000 (or any of the surgical systems described herein). The method 3010 may be implemented, at least in part, via the controller 3900 shown in FIGS. 6A and 6B (similarly described in FIG. 5 with reference to controller 2900). Instruction for operating the controller and the execution of the methods and processes 3010 can be stored in associated memory device(s) which are configured to perform the variety of computer implemented functions (e.g., performing the methods, steps, calculations and the like and storing relevant data as disclosed herein).

As depicted at 3012, the controller 3900 operates the actuator system (e.g., the manipulator unit 3200) in a variety of modes. These modes can include a teleoperated input following control mode, a limited teleoperated input following control mode, an instrument locked control mode, an instrument release control mode, along with other suitable operational control modes. References are made herein to first control modes and second control modes. In accordance with the embodiments, discussed herein, the system can switch between these control modes according to various algorithmic constraints. In some embodiments, the first control mode can be a locked control mode. In some embodiments, the first control mode can be a teleoperated input following control mode. In some embodiments, the first control mode can be a limited teleoperated input following control mode. In some embodiments, the second control mode is an instrument release mode.

In some embodiments, instrument locked control mode can be instituted in response to the system encountering an error. The error can be due to an unresponsive end effector due to a jam or other unexpected restriction in movement. In some embodiments, the instrument locked control mode can be instituted in response to the surgeon actively engaging the instrument locked control mode. The instrument locked control mode can lock inner and outer degrees of freedom of the various joints of the surgical instrument to limit unintended movement.

In some embodiments the teleoperated input following control mode can include various different modes of operation that allow the surgeon to guide the instrument 3400 from the control unit (e.g., control unit 2100 illustrated in FIG. 5 and control unit 1100 illustrated in FIGS. 1 and 2). At least one of these various different modes of operation provides control of the instrument 3400 based on input by the surgeon. Input at the control unit allows the surgeon to guide the instrument 3400. Under standard or typical conditions of the surgical system, the surgeon is allowed to provide input to perform surgery on the patient with the instrument 3400. The teleoperated input following control mode is an operation mode of the surgical system 3000 that contrasts with, for example, modes in which the surgical system 3000 is operating under an error state in which the surgical system 3000 is not performing as directed by the surgeon from the control unit. In some embodiments, the surgical system 3000 can include a step to operate the system in the teleoperated input following control mode prior to operating the system in the instrument locked mode.

In some embodiments the limited teleoperated input following control mode can include various different modes of operation that allow the surgeon to guide the instrument 3400 from the control unit (e.g., control unit 2100 illustrated in FIG. 5 and control unit 1100 illustrated in FIGS. 1 and 2). This movement can be limited by reducing torque or locking certain joints to restrict their degrees of freedom (dofs.). In some embodiments, the instrument can be operated in the limited teleoperated input following control mode after the system has operated in the instrument release mode. This can help protect the manual input mechanism from damage by locking dofs, that the manual input mechanism controls.

As depicted at 3012, the controller 3900 initial command to operate in first control mode. If the initial command has already been processed, the system can continue to operate in the first control mode (e.g., instrument locked mode or teleoperated input following control mode) or in some embodiments, it can operate in an alternative control mode (e.g., limited teleoperated input following control mode). At 3012, the controller 3900 commands the actuator system to operate in the first control mode, allowing the surgeon to proceed with a typical surgical procedure.

In accordance with some embodiments, the surgical system 3000 monitors the functionality and activity of one or more components of the surgical system 3000. For example, the surgical system 3000 can monitor kinematic parameters of components in the manipulator unit 3200 or the instrument 3400 (or both). These kinematic parameters can be monitored by receiving signals from sensors or other components that include values which reflect individual kinematic parameters of the various mechanical components of the manipulator unit 3200 or the instrument 3400 (or both).

As depicted at 3014, the controller 3900 receives a signal containing a kinematic parameter value (as an example, the kinematic parameter signal function 2910 of controller 2900 receives the kinematic parameter values as illustrated in FIG. 5). The kinematic parameter value can be related to or indicative of a kinematic parameter of a first movable operating component. For example, as described below, the kinematic parameter can be an angular orientation of the drive disk 3312. In such an example, the kinematic parameter value can be a voltage that corresponds to the angular orientation (in radians) from a reference position. As depicted at 3016, the controller 3900 receives another signal containing another kinematic parameter value. This second kinematic parameter value includes information related to the second movable operating component. For example, as described below, the kinematic parameter can be an angular position of the drive disk 3322, and the kinematic parameter value can be a voltage that corresponds to the angular position (in radians) from a reference position. In some embodiments, individual values from the signal can be utilized. In some embodiments, multiple values from each signal can be utilized. In some embodiments, a time-limited segment of the signal can be utilized. In some embodiments, the entire transmitted signal can be utilized.

The movable operating components can be located in the manipulator unit 3200 or the instrument 3400. The source of the signal can be located in the manipulator unit 3200 or the instrument 3400. In some embodiments, either one or both of the movable operating components and the source of the signal can be located in the same surgical system component, e.g., the manipulator unit 3200. In other embodiments, the source of the signal and one or both of the movable operating components can be in different surgical system components, e.g., the signal source can be in the manipulator unit 3200 while the movable operating component can be in the instrument 3400. For example, the signal source can be the electromechanical drive 3310 with the kinematic parameter value contained within the signal source pertaining to a kinematic parameter of tool drive member 3710 located in the mechanical structure 3700 of the instrument 3400. In an example in which the electromechanical drive 3310 is an electric motor, current draw, voltage, armature position, angular velocity, back EMF, or other electric motor information and characteristics can all be measured and sent to controller 3900 as a signal. These measurements can be used to derive, for example, the speed, the position, or the torque applied to components in the surgical instrument 3400 such as an input disk or a capstan or any other mechanism used to drive an end effector. Thus, information about the movable operating component in the instrument 3400 is able to be conveyed to the controller 3900 by signals from the electromechanical drive in the manipulator unit 3200 that drives the movable operating component. Eliminating sensors from the instrument 3400 allows for fewer parts and an easier-to-manufacture instrument 3400 that is overall more inexpensive. With instrument 3400 more inexpensive, surgeons have greater flexibility in choosing to discard the used instrument.

As depicted at 3018, a compared value is determined. This compared value is based on a calculation derived from both the kinematic parameter values relevant to the separate movable operating components. By evaluating the kinematic parameter value from one movable component with reference to a separate kinematic parameter value from another movable component, a separate compared value is obtained that can be used to determine if the two movable components are being driven in sync (e.g., by a connected mechanism such as the manual input 3863) or separately (e.g., by separately connected mechanisms such as the different tool members within an end effector). As indicated previously, the manual input mechanism 3860 can drive both movable components 3710 and 3720 together. Similar changes in each of the kinematic parameter values would indicate simultaneous changes in the separate movable components. The simultaneous changes would likely be caused by a mechanism that engages both movable components together, like the manual input mechanism 3860, whereas feedback from the end effector 3460 (e.g., a back drive movement caused by one or more tool members of the end effector) would not drive the two movable components simultaneously and would therefore show disparate changes in the kinematic parameter values of the movable components. In one example, the compared value is defined by taking an average of the two values and utilizing that average as the compared value. In another example, the compared value is defined by taking the difference between the first kinematic parameter value and the second kinematic parameter value. The smaller the difference between the two values is, the more likely that the two movable components are moving in sync.

While the kinematic parameter value has been described as being singular, it should be understood that multiple values can be used from each signal and the kinematic parameter value can be just one of the multiple values. In some embodiments, a larger portion of the values contained in each of the signals can be used. This value set (also referred to as a plurality of values) from each source are compared as a function of time to form the compared value, compared signal, or a compared value string. In such examples, the compared value string can be calculated as the common mode of the two signals as a function of time, or, in some embodiments, the compared value can be calculated as the differential mode of the two signals as a function of time. In some embodiments, both the common mode and the differential mode can be assessed to determine the likelihood of synchronized changes or disparate changes to the two movable components. In other embodiments, different calculations and methods of signal analysis can be used to determine if the signals are sufficiently similar to determine that the movable components are being driven together be a mechanism in common or if the signals are sufficiently dissimilar to determine that the movable components are being driven separately such as by separate mechanisms (e.g., end effector feedback). Examples of other calculations usable as signal analysis could include differentiating the signals to determine if the different signals share similar changes in curve slope. Such an analysis might be irrespective of magnitude of measurements but instead focused on commonality in the rate of change between the kinematic parameters of the two movable components. Other suitable calculations to determine similarities and differences in the signals are also usable.

In some embodiments, the calculations, analysis, or processing of the kinematic parameter values, signals or value sets can be performed by the kinematic parameter signal function within the controller 3900. An example of such function and controller is the kinematic parameter signal function 2910 of controller 2900 as illustrated in FIG. 5.

As depicted at 3020, the controller 3900 compares the compared value (e.g., the differential mode of the signals) to a defined value range and determines if the compared value is within the defined range. For example, if the compared value is the differential mode of the signals, the value range can include a range centered around 0. If the two signals are identical, then the differential mode will be 0. Due to tolerances in the mechanical structure of the different movable components and noise (e.g., movement of the end effectors), the signals associated with the different powertrains may be different even though they are both being driven in a certain instance by the manual input. As such, a range around 0 can still indicate use of the manual input. In one embodiment, the range can be about 20% of the total signal amplitude above and below zero. For example, in a rotary mechanism if the total movement is 0.5 radians, then the value range might be from −0.1 to 0.1 radians. In another embodiment, the range can be about 10% of the total signal amplitude above and below zero. For example, in a rotary mechanism if the movement is 0.5 radians, then the value range might be from −0.05 to 0.05 radians. If the compared value is outside of this range, then a determination can be made that the manual input is not being used.

In another example, if the compared value is the common mode of the signals, the value range can be centered around a range of an expected maximum amplitude of the signal. If the two signals are substantially identical, then the common mode will match the curve of either signal. Due to tolerances in the mechanical structure of the different movable components and noise (e.g., movement of the end effectors), the signals associated with the different powertrains may be different even though they are both being driven in a certain instance by the manual input. As such, evaluating the common mode against a range around the maximum amplitude of the signal can still indicate use of the manual input. In one embodiment, the range can be about 20% of the total signal amplitude below the max amplitude of the signal. For example, in a rotary mechanism if the total movement is 0.5 radians, then the value range might be from 0.4 to 0.6 radians (or plus/minus 0.1 radians). In another embodiment, the range can be about 10% of the total signal amplitude below the max amplitude of the signal. For example, in a rotary mechanism if the movement is 0.5 radians, then the value range might be from 0.45 to 0.55 radians. In some embodiments, the range can be open ended for example, a minimum threshold can be set (e.g., 45 radians) and anything in excess of that minimum threshold would satisfy the condition.

To further illustrate the method, FIGS. 8A-8I are examples of various sets of values as a function of time representing the kinematic parameter of a first movable component (solid line) and the kinematic parameter of a second movable component (dashed line) for three different operational scenarios. FIG. 8A illustrates an example first signal (solid line; representing the kinematic parameter of the first movable component) and second signal (dashed line; representing the kinematic parameter of the second movable component). FIG. 8B illustrates the common mode of the two signals of FIG. 8A and FIG. 8C illustrates the differential mode of the signals of FIG. 8A. Here, the common mode signal is within the range shown in shading. Additionally, the curve of FIG. 8A matches the shape and characteristics of the curve of FIG. 8B (i.e., the peaks have a similar magnitude and occur at similar points in time). As such, the controller 3900 would make a determination that the manual input is in use and, in some embodiments, the method would include commanding the actuator system to operate in an instrument release mode, at 3024. Similarly, as shown in FIG. 8C, the differential signal is within the range shown in shading. As such, the controller 3900 would make a determination that the manual input is in use, and, in some embodiments, the method would include commanding the actuator system to operate in an instrument release mode, at 3024.

FIG. 8D illustrates a second set of signals at a second operational scenario. Specifically, a first signal representing the kinematic parameter of the first movable component is shown as a solid line and a second signal representing the kinematic parameter of the second movable component is shown as a dashed line. Here, the curves mirror images of one another. FIG. 8E shows a common mode of the two signals and FIG. 8F shows a differential mode of the two signals. The entirety of the common mode is out of the defined range. As such, the controller 3900 would make a determination that the manual input is not use. In some embodiments, the method would include commanding the actuator system to operate in a first control mode, at 3022. Referring to FIG. 8F, significant portions of the differential mode extend outside of the defined range. As such, the controller 3900 would make a determination that the manual input is not in use. In some embodiments, the method would include commanding the actuator system to operate in a first control mode, at 3024.

In some embodiments, both the common mode and the differential mode must satisfy the condition (e.g., fall within the defined range) to determine that there is use of the manual user input. For example, as described for the first example of FIGS. 8A-8C both the common mode and the differential mode were within the defined range. Similarly, in the second example of FIGS. 8D-8F, both the common mode and the differential mode were outside of the defined range. In other embodiments, if either the common mode or the differential mode satisfies the condition (e.g., falls within the defined range) then is the controller can determine that there is use of the manual user input. In yet other embodiments, if either the common mode or the differential mode does not satisfy the condition (e.g., falls outside the defined range) then is the controller can determine that there is no use of the manual user input.

FIG. 8G illustrates a third example, with a first and second signal. Here the signals look similar but are significantly shifted in time. Specifically, a first signal representing the kinematic parameter of the first movable component is shown as a solid line and a second signal representing the kinematic parameter of the second movable component is shown as a dashed line. FIG. 8H shows a common mode of the two signals and FIG. 8I shows a differential mode of the two signals. Here, the common mode is more out of the defined range than it is in the first example (see FIG. 8B), but it is still none-the-less within range. As such, the controller 3900 could make a determination that the manual input is in use. In some embodiments, the method would include commanding the actuator system to operate in a first control mode, at 3022. Referring to FIG. 8I, here portions of the differential mode extend out of the range. As such, the controller 3900 would make a determination that the manual input is not in use. In some embodiments, the method would include commanding the actuator system to operate in a first control mode, at 3024. Because there are contradictory indications between the common mode and differential mode a decision regarding risk tolerance can be made. In some embodiments, the risk tolerance favors inclusive indication of manual input use, then the system can react as though manual input has been used even though the differential mode indicates it has not. In some embodiments, the risk tolerance favors under inclusion of indications of manual input use, then the system can react as though manual input has not been used, even though the common mode has indicated that it has been used. In some embodiments, the system can operate as an absolute system requiring an indication that both the common mode and the differential mode indicate use. In such an embodiment, the system can treat this as no manual input use because at least some of the differential mode signal is out of the range. In some embodiments, the ranges can be tuned to modulate the algorithms inclusivity based on risk tolerance. For example, low a differential mode range and high common mode threshold is more risk averse and the opposite for each range would be more inclusive.

While these examples are provided in terms of position and, more specifically, angular orientation, it is appreciated that other kinematic parameters are contemplated herein and other mechanisms such as linear sliding mechanisms are contemplated herein as well.

Depending on the conditions of the compared value relative to the defined value range, the controller 3900 can execute different instructions. For example, in response to a condition in which the compared value is outside the defined value range, as depicted at 3022, the control system continues to operate the actuator system in a first control mode. In response to a condition in which the compared value is within the defined value range, as depicted at 3024, the control system operates the actuator system in an instrument release mode.

In accordance with some embodiments, the instrument release mode includes surgical system 3000 operational modes that limit the ability of the user input unit to drive the instrument 3400. In particular, the controller 3900 would limit the ability of either electromechanical actuator 3310 or 3320 to overpower the use of the manual input mechanism 3860. Generally, the first actuator powertrain is operable to drive the first movable operating component of the instrument (e.g., 3846, 3847, 3710, etc.). However, in accordance with some embodiments, in instrument release mode the actuation applied by the electromechanical actuator to the first powertrain is limited to merely applying a dampening torque in the first movable operating component. Similarly, the actuation applied by the electromechanical actuator to the second powertrain is limited to merely applying a dampening torque in the second movable operating component of the instrument (e.g., 3848, 3849, 3720, etc.). The first dampening torque is less than a torque applied to the first movable component by movement of the manual control input. Similarly, the second dampening torque is less than a torque applied to the second movable component by movement of the manual control input. In accordance with some embodiments, the dampening torque is the minimal sufficient torque to hold the surgical instrument in place and less than the torque applied by the manual input device.

In accordance with some embodiments, latched kinematic parameter values are obtained. The latched values can be used as reference values for subsequent kinematic parameter values included in any subsequently acquired value sets received by the controller. Once latched, the latched kinematic parameter values can be used as a reference to the subsequently acquired values such that changes in the kinematic parameters of the system can be observed. In some embodiments, the latch is reset on condition that the compared value is outside of the defined range. In some embodiments, the latch is reset on condition that the compared value is within the defined range. In such embodiments, the controller 3900 places the system in instrument release mode. The controller can additionally or alternatively log the use of the manual input device 3860. In some embodiments, the system can return to normal operation (e.g., teleoperated input following control mode or instrument lock mode) after the jam on the end effector is cleared by using the manual input device 3860. In such embodiments, after resetting or continuing with the instrument control mode (e.g., teleoperated input following control following, instrument lock or other), the algorithm can begin again by setting the latch. In other embodiments, the system may require that the instrument 3400 be replaced after the jam on the end effector is cleared. In some embodiments, the instrument can be put into a limited teleoperated following control mode if the user attempts to return to teleoperated following control mode after the manual release has been used. In the limited teleoperated following control mode, the system locks certain degrees of freedom (e.g., the ones coupled to the manual release mechanism) while still allowing teleoperation to prevent damage to the manual release mechanism.

In accordance with some embodiments, a variety of instrument types can be installed on the manipulator unit 3200. For example, one instrument 3400 can include clamping end effector tools. Another instrument can include cutting end effector tools. Another instrument can include tissue manipulation end effector tools. Any suitable tool is contemplated herein. The different instrument types can have different range values. As such, the controller 3900 can detect the instrument type and apply the correct range value for the instrument. In some embodiments, the method includes determining if an instrument operatively coupled to the actuator system is one of a first instrument type or a second instrument type different from the first instrument type. In some embodiments, the controller 3900 then commands the actuator system to operate in the instrument release mode only on a condition in which the instrument type is the first instrument type and on the condition in which the compared value is within a defined value range for the first instrument type. A second instrument type can subsequently or alternatively be loaded on the manipulator device. The second instrument type is different from the first instrument type. The controller 3900 then commands the actuator system to operate in the instrument release mode only on a condition in which the compared value is within a defined value range for the second instrument type. In some embodiments, the instrument defined value range is different between the first instrument defined value range and the second instrument defined value range.

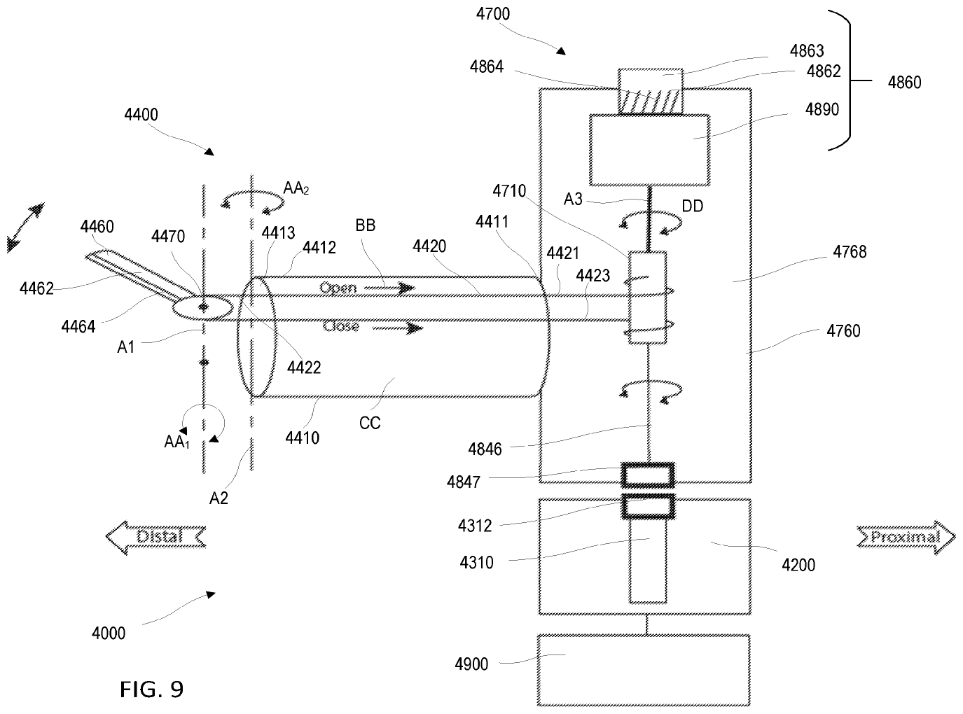

FIG. 9 is a schematic illustration of a portion of a surgical system 4000 according to an embodiment. The surgical system 4000 includes a surgical instrument 4400, a manipulator unit 4200, and a controller 4900. The manipulator unit 4200 (which functions as an actuator system) is in communication with the controller 4900 such that the controller 4900 can send and receive signals from the manipulator unit 4200. For example, the controller 4900 can receive input control signals from the input control unit (such as user control unit 1100 in FIG. 1 or user control unit 2100 in FIG. 5) based on surgeon input (receivable for example at the control input function 2908 of controller 2900 in FIG. 5). The controller 4900 then sends control signals to the manipulator unit 4200 (as an example, the device control function 2914 of controller 2900 sends the control signals as illustrated in FIG. 5) which, in turn, drives the surgical instrument 4400. In this manner the manipulator unit 4200 (and any other manipulator units described herein) functions as an actuator system that actuates the surgical instrument 4400. The surgical instrument includes a mechanical structure 4700 having a manual drive mechanism 4860. As discussed in more detail below the manual drive mechanism 4860 drives a powertrain that includes one or more of a tool drive member 4710, input member 4846, and input disk 4847. While shown in FIG. 9 with a single powertrain, multiple power trains can be included (see e.g., FIGS. 6A, 6B, and 14-25B). These powertrain components are also variously referred to as movable components. As shown in FIG. 9, the manual drive mechanism 4860 includes mechanical components to drive the powertrain by external forces from the user by a single actuation of the manual input device 4863. The first powertrain can also be driven by electromechanical drive 4310. Drive disk 4312 can engage with input disk 4847. Due to the engagement between the disks, the manual drive mechanism 4860 is also sufficiently mechanically coupled with electromechanical drive 4310 to drive (or cause some movement of) electromechanical drive 4310. Because of the mechanical coupling between the electromechanical drive 4310, via the powertrain in the instrument 4400, the electromechanical drive 4310 can produce a signal that reflect one or more kinematic parameters of the movable component (e.g., 4710, 4846, 4846, etc.) in the surgical instrument 4400. This allows the controller 4900 to recognize use of the manual drive 4863 and change operation modes to accommodate the use of the manual drive 4863.

As shown in FIG. 9, in some embodiments, the medical device 4400 includes a shaft 4410, a tension member 4420, an end effector 4460, and the mechanical structure 4700. The mechanical structure 4700 functions to receive one or more motor or manual input forces or torques and mechanically transmit the received forces or torques to move the end effector 4460. For example, as described above the manipulator unit 4200 can include one or more electric motors (which function as an electromechanical drive, e.g., the electromechanical drive 4310) to provide an input to the mechanical structure 4700, which in turn transmits the input via the tension member 4420 to control the end effector 4460. Specifically, the mechanical structure includes a chassis 4768, a tool drive member 4710 and a manual drive mechanism 4860. The chassis 4768 provides the structural support for mounting or supporting and aligning the components of the mechanical structure 4700. For example, openings, protrusions, mounting brackets and the like can be defined in or on chassis 4768. In some embodiments, the chassis 4768 can include multiple portions, such as an upper chassis and a lower chassis. In some embodiments, a housing 4760 can optionally enclose at least a portion of the chassis 4768.

The tool drive member 4710 is mounted to the mechanical structure 4700 (e.g., within the housing 4760) via a first tool drive member support member (not shown). For example, the first tool drive member support member can be a mount, shaft, or any other suitable support structure to secure the tool drive member 4710 to the mechanical structure 4700. The tool drive member 4710 includes (or is coupled to) a first input member 4846 and an input disk 4847. The first input member 4846 can be connected to and receive mechanical input from the electromechanical drive 4310. The tool drive member 4710 can be operable to be rotated about an axis A3 in a direction DD, as shown in FIG. 9.

The manual drive mechanism 4860 includes manual input device 4863, a manual drive input member 4862, a signature generating mechanism 4864, and a manual drive coupling member 4890. The manual drive input member 4862 is mechanically connected to the manual input device 4863. The manual input device 4863 includes a portion that is exposed to the exterior of the medical device 4400. The user can engage the manual input device 4863 and manipulate the manual drive mechanism 4860, thereby manipulating the end effector 4460. The exposed portion of the manual input device 4863 can include any suitable structure for receiving the user's input force. For example, the manual input device 4863 can include a rotatable wheel, a rotatable knob, a push button, a slide, or other suitable mechanical structures that receive the user's input force and allows the manual drive mechanism 4860 to translate the user's input motion to an input on the tool drive members 4710 and thereby manipulate the end effector 4460.

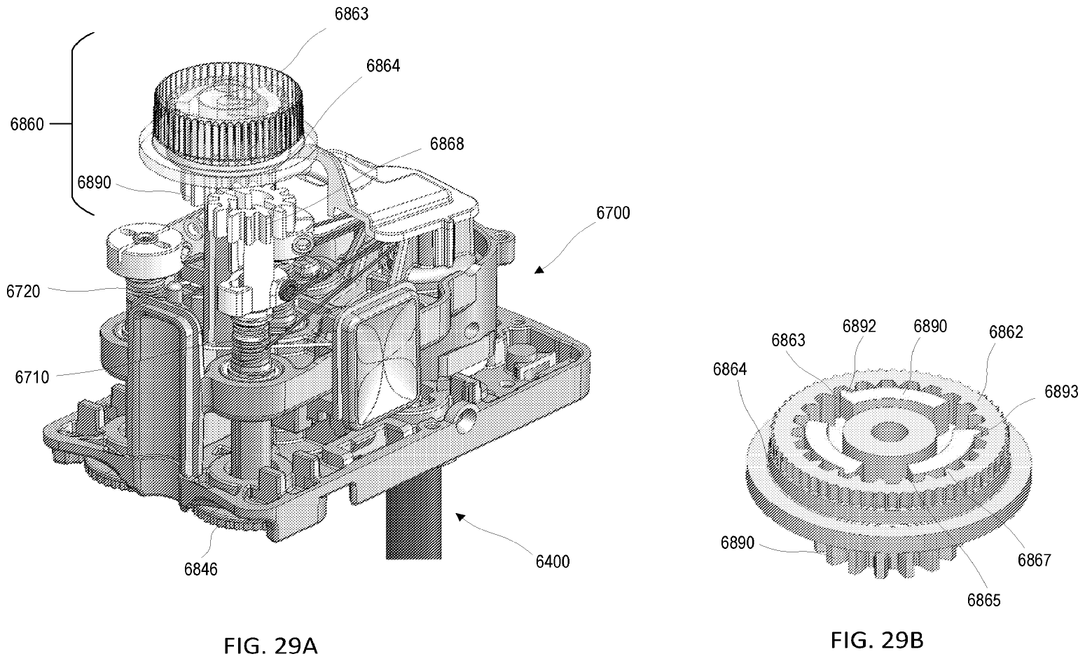

The manual drive mechanism 4860 also can produce a signature detectable via the electromechanically drive 4310 via the signature generating mechanism 4864. As discussed in more detail below; the signature generating mechanism 4864 provides a distinctive signal to the sensor readable by the controller 4900 to indicate that the manual drive mechanism 4860 is in use. To do this, the signature generating mechanism 4864 produces a distinctive output of a kinematic parameter that is unlikely to be reproduced by feedback from the end effector 4460. For example, (as discussed in greater detail with respect to FIG. 29), the manual drive mechanism 4860 can include a clutch mechanism. The clutch mechanism can cause the torque on or the position of the tool drive member 4710 to vary in a defined or otherwise known manner. For example, by allowing the clutch to increase torque and then slip, resulting torque or position spikes can occur in tool drive member 4710. These resulting torque or position spikes can then communicate to controller 4900 by receiving kinematic parameter values from the sensor.

The manual drive coupling member 4890 is connected to the manual drive input member 4862. The manual drive coupling member 4890 transmits the user's force from the manual drive input member 4862 to at least one tool drive member 4710 (in other embodiments the coupling member can transmit the user's force to a second tool drive member see FIGS. 6A, 6B and 14-25B). In some embodiments, the manual drive coupling member 4890 allows the input force to be transmitted from the manual drive input member 4862 toward the tool drive member 4710, but not from the tool drive member 4710 toward the manual drive input member 4862. Said another way, in some embodiments, the manual drive coupling member 4890 does not allow movement of the tool drive member 4710 to cause movement of the manual drive input member 4862.

The manual drive coupling member 4890 can include any suitable structure or components to perform the functions described herein. For example, in some embodiments, the manual drive coupling member 4890 can include a gear member or set of gears that can be engaged and disengaged from the tool drive member 4710. In other embodiments clutches, tension members, hydraulics, slidable linkages, or any other suitable mechanism for transmitting force from the user to the tool drive members some of which are discussed herein and others that a person of ordinary skill in the art would apply based on the disclosure provided herein.

The tension member 4420 includes a first proximal portion 4421, a second proximal portion 4423 and a distal portion 4422. The first proximal portion 4421 and the second proximal portion 4423 are each coupled to the mechanical structure 4700, and the distal portion 4422 is coupled to the end effector 4460. The shaft 4410 includes a proximal end portion 4411 and a distal end portion 4412 and defines a passageway 4413 that extends lengthwise through the shaft between the proximal and distal end portions. In accordance with various embodiments, the tension member includes any member suitable tension member for transmitting force between the tool drive member 4710 and the end effector. For example, the tension member can include one or more of a cable, band, strap, string, wire, tube, rod, etc. The tool drive member 4710 includes one or more of capstans, winches, spools, or other suitable devices for containing, controlling, taking up, and dispensing the tension member 4420.

As shown in FIG. 9, the tool drive member and the tension member can be mechanically connected in a self-antagonistic drive arrangement. However, it is appreciated that in other embodiments, the tool drive member and the tension member can be mechanically connected in an antagonistic drive arrangement along with an additional tool drive member (compare to FIG. 6A and FIG. 6B). However, for clarity, the self-antagonistic drive is discussed herein.

The end effector 4460 is rotatably coupled to the distal end portion 4412 of the shaft 4410 and includes at least one tool member 4462. The medical device 4400 is configured such that movement of the first proximal portion 4421 and the second proximal portion 4423 of the tension member 4420 produces movement of the tool member 4462 about a first axis of rotation A1 (which functions as the yaw axis; the term yaw is arbitrary), in a direction of arrows AA1. In some embodiments, the medical device 4400 can include a wrist assembly including one or more links that couples the end effector 4460 to the distal end portion 4412 of the shaft 4410. In such an embodiment, movement of the first proximal portion 4421 and the second proximal portion 4423 of the tension member 4420 can also produce movement of the wrist assembly about a second axis of rotation A2 (which functions as the pitch axis) or both movement of the wrist assembly and the end effector 4460. See, for example, U.S. provisional application No. 63/233,904 incorporated by reference above.

The tool member 4462 includes a contact portion 4464 and a drive pulley 4470. The contact portion 4464 is configured to engage or manipulate a target tissue during a surgical procedure. For example, in some embodiments, the contact portion 4464 can include an engagement surface that functions as a gripper, cutter, tissue manipulator, or the like. In this manner, the contact portion 4464 of the tool member 4462 can be actuated to engage or manipulate a target tissue during a surgical procedure. The tool member 4462 (or any of the tool members described herein) can be any suitable medical tool member. Moreover, although only one tool member 4462 is shown, in other embodiments, the medical device 4400 can include two or more moving tool members that cooperatively perform gripping or shearing functions.

As shown in FIG. 9, in a self-antagonistic arrangement, the tension member 4420 is routed from the mechanical structure 4700 to the end effector 4460 and then back to mechanical structure 4700, and each individual end of the tension member 4420 is coupled to the tool drive member 4710 (in an antagonistic arrangement each end would couple to different tool drive members) of the mechanical structure 4700.

The two ends of the tension member 4420 that are associated with opposing directions of a single degree of freedom of the tool member 4462 are connected to the same tool drive member 4710. This arrangement allows for singular control of the movement of (e.g., pulling in or paying out) each of the ends of the tension member 4420 by the same tool drive member. The mechanical structure 4700 produces movement of the tension member 4420, which operates to produce the desired articulation movements (pitch, yaw, or grip) at the end effector 4460. Accordingly, as described herein, the mechanical structure 4700 includes components and controls to move the first proximal portion 4421 of the tension member 4420 via the tool drive member 4710 in a first direction (e.g., a proximal direction) and a second opposite direction (e.g., a distal direction).

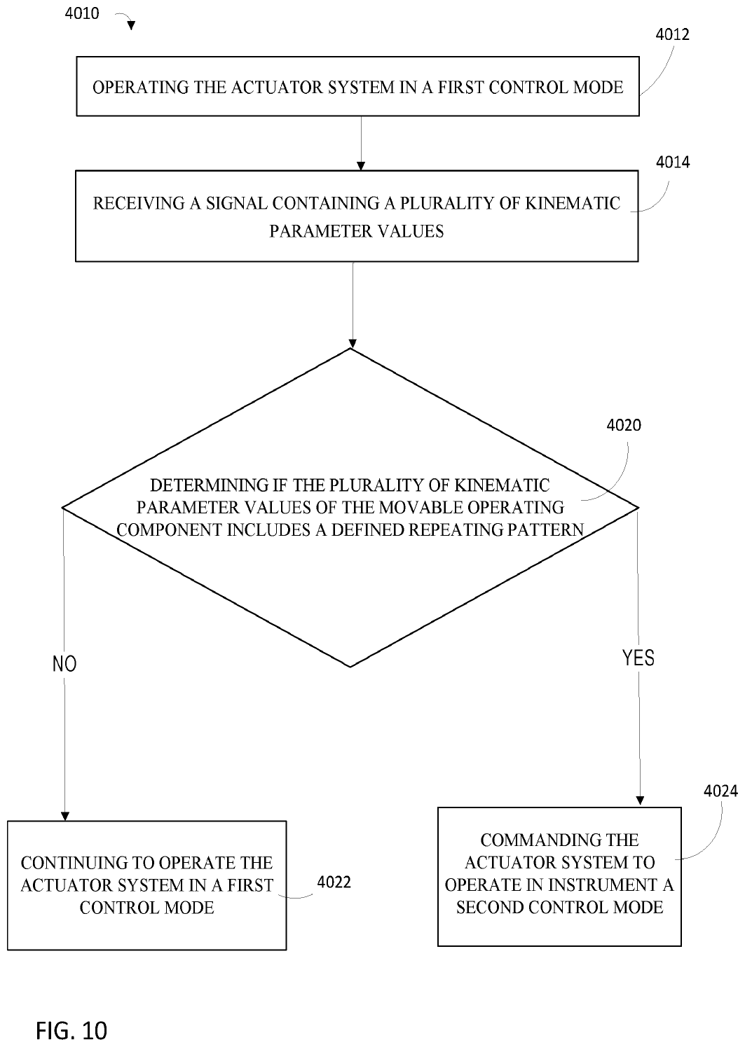

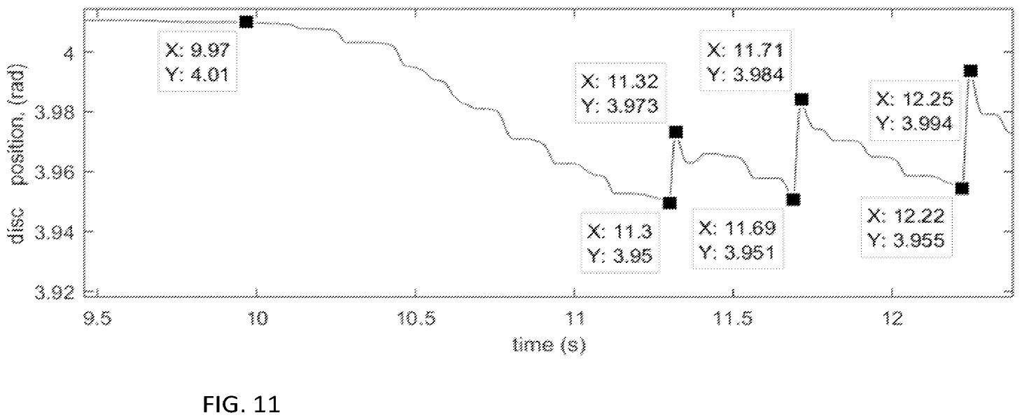

FIG. 10 is a flow chart of a control method 4010 for a surgical system according to an embodiment. The method 4010 can, in an embodiment, be performed via the surgical system as described with reference to any of the systems shown in FIGS. 1-5 and 9, or any other systems herein. However, it should be appreciated that in various embodiments, aspects of the method 4010 may be accomplished via additional embodiments of the surgical system or components thereof, such as instrument 6400 (shown in FIG. 29). FIG. 11 shows an example of a signal and signal processing for determining control of the surgical system 4000 (or any of the surgical systems described herein). The method 4010 may be implemented, at least in part, via the controller 4900 shown in FIG. 9 (similarly described in FIG. 5 with reference to controller 2900). Instruction for operating the controller and the execution of the methods and processes 4010 can be stored in associated memory device(s) which are configured to perform the variety of computer implemented functions (e.g., performing the methods, steps, calculations and the like and storing relevant data as disclosed herein.

As depicted at 4012, the controller 4900 operates the actuator system (e.g., the manipulator unit 4200) in a variety of modes. These modes can include a teleoperated input following control mode, a limited teleoperated input following control mode, an instrument locked control mode, an instrument release control mode, along with other suitable operational control modes. References are made herein to first control modes and second control modes. In accordance with the embodiments, discussed herein, the system can switch between these control modes according to various algorithmic constraints. In some embodiments, the first control mode can be a locked control mode. In some embodiments, the first control mode can be a teleoperated input following control mode. In some embodiments, the first control mode can be a limited teleoperated input following control mode. In some embodiments, the second control mode is an instrument release mode.

In some embodiments, instrument locked control mode can be instituted in response to the system encountering an error. The error can be due to an unresponsive end effector due to a jam or other unexpected restriction in movement. In some embodiments, the instrument locked control mode can be instituted in response to the surgeon actively engaging the instrument locked control mode. The instrument locked control mode can lock inner and outer degrees of freedom of the various joints of the surgical instrument to limit unintended movement.

In some embodiments the teleoperated input following control mode can include various different modes of operation that allow the surgeon to guide the instrument 4400 from the control unit (e.g., control unit 2100 illustrated in FIG. 5 and control unit 1100 illustrated in FIGS. 1 and 2). At least one of these various different modes of operation provides control of the instrument 4400 based on input by the surgeon. Input at the control unit allows the surgeon to guide the instrument 4400. Under standard or typical conditions of the surgical system, the surgeon is allowed to provide input to perform surgery on the patient with the instrument 4400. The teleoperated input following control mode is an operation mode of the surgical system 4000 that contrasts with, for example, modes in which the surgical system 4000 is operating under an error state in which the surgical system 4000 is not performing as directed by the surgeon from the control unit. In some embodiments, the surgical system 4000 can include a step to operate the system in the teleoperated input following control mode prior to operating the system in the instrument locked mode.

In some embodiments the limited teleoperated input following control mode can include various different modes of operation that allow the surgeon to guide the instrument 4400 from the control unit (e.g., control unit 2100 illustrated in FIG. 5 and control unit 1100 illustrated in FIGS. 1 and 2). This movement can be limited by reducing torque or locking certain joints to restrict their degrees of freedom (dofs.). In some embodiments, the instrument can be operated in the limited teleoperated input following control mode after the system has operated in the instrument release mode. This can help protect the manual input mechanism from damage by locking dofs. that the manual input mechanism controls.

As depicted at 4012, the controller 4900 initial command to operate in first control mode. If the initial command has already been processed, the system can continue to operate in the first control mode (e.g., instrument locked mode or teleoperated input following control mode) or in some embodiments, it can operate in an alternative control mode (e.g., limited teleoperated input following control mode). At 4012, the controller 4900 commands the actuator system to operate in the first control mode, allowing the surgeon to proceed with the surgical procedure.

In accordance with some embodiments, the surgical system 4000 monitors the functionality and activity of one or more components of the surgical system 4000. For example, the surgical system 4000 can monitor kinematic parameters of components in the manipulator unit 4200 or the instrument 4400 (or both). These kinematic parameters can be monitored by receiving signals from sensors or other components that include values which reflect individual kinematic parameters of the various mechanical components of the manipulator unit 4200 or the instrument 4400 (or both).

As depicted at 4014, the controller 4900 receives a signal containing a kinematic parameter value set. The kinematic parameter value set can be related to or indicative of a kinematic parameter of a first movable operating component. For example, as described below, the kinematic parameter can be an angular orientation of the drive disk 4312. In such an example, the kinematic parameter value set can be a voltage that corresponds to the angular orientation (in radians) from a reference position. In contrast to the embodiment, described with respect to FIGS. 6A-8I, some embodiments (as illustrated in FIGS. 9-11) utilize a single signal source to identify usage of the manual input 4860. In some embodiments, individual values from the signal can be utilized. In some embodiments, multiple values from the signal can be utilized. In some embodiments, a time-limited segment of the signal can be utilized. In some embodiments, the entire transmitted signal can be utilized.

While the surgical system 4000 can include multiple movable operating components, fewer than all of the movable operating components can be used to determine manual input 4860 use. In the embodiment shown in FIGS. 9-11, the kinematic parameter of a single movable operating component can be utilized to determine whether the manual input 4860 is in use. The movable operating component can be located in the manipulator unit 4200 or the instrument 4400. The source of the signal can be located in the manipulator unit 4200 or the instrument 4400. In some embodiments, either one or both of the movable operating component and the source of the signal can be located in the same surgical system component, e.g., the manipulator unit 4200. In other embodiments, the source of the signal and the movable operating component can be in different surgical system components, e.g., the signal source can be in the manipulator unit 4200 while the movable operating component can be in the instrument 4400. For example, the signal source can be the electromechanical drive 4310 with the kinematic parameter value set contained within the signal source pertaining to a kinematic parameter of tool drive member 4710 located in the mechanical structure 4700 of the instrument 4400. In an example in which the electromechanical drive 4310 is an electric motor, current draw, voltage, armature position, angular velocity, back EMF, or other electric motor information and characteristics can all be measured and sent to controller 4900 as a signal. These measurements can be used to derive, for example, the speed, the position, or the torque applied to components in the surgical instrument 4400 such as an input disk or a capstan or any other mechanism used to drive an end effector. Thus, information about the movable operating component in the instrument 4400 is able to be conveyed to the controller 4900 by signals from the electromechanical drive in the manipulator unit 4200 that drives the movable operating component. Eliminating sensors from the instrument 4400 allows for fewer parts and an easier-to-manufacture instrument 4400 that is overall more inexpensive. With instrument 4400 more inexpensive, surgeons have greater flexibility in choosing to discard the used instrument.

In accordance with various embodiments, the kinematic parameter value set includes a plurality of values. In some embodiments, a larger portion of the values contained in the signal can be used. In some embodiments, a smaller portion of the values contained in the signal can be used. These value sets (also referred to as a plurality of values) from the sensor can be evaluated as a function of time.

In some embodiments, the kinematic parameter value set can be evaluated. In some embodiments, the calculations, analysis, or processing of the kinematic parameter values, signals or value sets can be performed by the kinematic parameter signal function within the controller 4900. An example of such function and controller is the kinematic parameter signal function 2910 of controller 2900 as illustrated in FIG. 5.

In embodiments in which the kinematic parameter of a single movable component is used to determine manual input from the manual input mechanism 4860, the manual input mechanism can provide a signature or identifying information such that the kinematic parameter value set carries that identifying information to the controller 4900. In this way, use of the manual input mechanism 4860 can be distinguished from feedback from the end effector 4460 (e.g., a back drive movement caused by one or more tool members of the end effector) since the end effector 4460 feedback would not return the same identifying information. In one example, the identifying information is an identifiable pattern of a kinematic parameter that the end effector 4460 would be unlikely to return. In some embodiments, different analytical methods can be used to determine if the kinematic parameter value set includes the identifying information sufficiently to determine that the movable components are being driven by the manual input mechanism 4860 or in the absence of the identifying information it is determined that the movable components are being driven separately such as by a separate mechanism (e.g., end effector feedback). Examples of identifying information could include a repeating pattern. As indicated above, the manual input mechanism 4860 can include a signature generating mechanism 4864. The signature generating mechanism 4864 can produce a repeating pattern that can be detected at the sensor associated with the electromagnetic drive 4310.

As depicted at 4020, the controller 4900 determines if the kinematic parameter value set includes the signature (e.g., the repeating pattern). In accordance with various embodiments, the determination of the presence of the signature (e.g., the repeating pattern) can be based on a defined value range. For example, if a significant change in angular orientation (in radians) occurs over a short enough period of time in a repeated manner along a sufficiently consistent period it can be determined that the end effector did not cause the repeated change and instead it was caused by the manual input mechanism 4860. For example, if the movable component changes angular orientation by at least 0.02 radians at least three times in a row in less than 0.2 seconds each time, the pattern is determined to be present and the condition for putting the system in instrument release mode is satisfied. In some embodiments, the radians can be repeatedly changed by anywhere from 0.01 radians to 1 radian. In other embodiments, the radians can be repeatedly changed by anywhere from 0.01 radians to 0.1 radians. In other embodiments, the radians can be repeatedly changed by anywhere from 0.02 radians to 0.05 radians. In some embodiments, the changes in kinematic parameter (e.g., angular orientation in radians) can occur greater than two times in a row. In some embodiments, the changes occur 3-5 times in a row. In some embodiments, each change occurs in less than 1 second. In some embodiments, each change occurs in less than 0.5 seconds. While the examples discussed herein describe changes in angular orientation, other kinematic parameter changes are also contemplated herein. For example, in non-rotary systems translational changes can be patterned as used as a signature. In another example, changes in torque can be patterned and used as a signature. Non-pattern signatures are also contemplated herein. For example, a specific linear increase in speed (i.e., translational or angular change in position), a specific linear increase in torque, or similar changes. In another example of a non-pattern signature, non-repeating but defined changes in the kinematic parameter can be used. For example, the angular position could change by varying defined amounts such as 0.1 radians then 0.05 radians then 0.07 radians. Any suitable signature that is definable and distinguishes over incidental movement cause be the end effector is contemplated herein. Due to tolerances in the mechanical structure of the different movable components and noise (e.g., movement of the end effectors), the signature is different enough to identify use of the manual input mechanism 4860 despite also being driven in part by incidental impact between the end effector and tissue while also using the manual input mechanism 4860.

To further illustrate the method, FIG. 11 is an example of a set of values as a function of time representing the kinematic parameter of a movable component (solid line). FIG. 11 illustrates an example first signal (solid line; representing the kinematic parameter of the movable component). As shown in FIG. 11, a series of three spikes in disc position change (shown in radians) occurs between 11.3 and 11.32 seconds, 11.69 and 11.71 seconds, and 12.22 and 12.25 seconds. When assessed against a pattern that includes at least 0.02 radian spikes in less than 0.04 second intervals and repeated on a period of between 0.2 seconds and 0.6 seconds, the pattern shown in FIG. 11 would cause the controller 4900 to make a determination that the manual input is in use. This would cause the controller 4900 to stop the first control mode and start the instrument release mode.

While these examples are provided in terms of position and, more specifically, radial position, it is appreciated that other kinematic parameters are contemplated herein and other mechanisms such as linear sliding mechanisms are contemplated herein as well.

Depending on the conditions of the compared value relative to the defined value range, the controller 4900 can execute different instructions. For example, in response to a condition in which the compared value is outside the defined value range, as depicted at 4022, the control system continues to operate the actuator system in a first control mode. In response to a condition in which the compared value is within the defined value range, as depicted at 4024, the control system operates the actuator system in an instrument release mode.

In accordance with some embodiments, the instrument release mode includes surgical system 4000 operational modes that limit the ability of the user input unit to drive the instrument 4400. In particular, the controller 4900 would limit the ability of electromechanical actuator 4310, along with any other electromechanical actuators, to overpower the use of the manual input mechanism 4860. Generally, the first actuator powertrain is operable to drive the first movable operating component of the instrument. However, in accordance with some embodiments, in instrument release mode, the actuation applied by the electromechanical actuator to the first powertrain is limited to merely applying a dampening torque in the first movable operating component. Similarly, the actuation applied by any additional electromechanical actuator to any additional powertrain is limited to merely applying a dampening torque in the additional movable operating components of the instrument. The first dampening torque is less than a torque applied to the first movable component by movement of the manual control input. Similarly, the second dampening torque is less than a torque applied to the second movable component by movement of the manual control input. In accordance with some embodiments, the dampening torque is the minimal sufficient torque to hold the surgical instrument in place and less than the torque applied by the manual input device.

In accordance with some embodiments, a latched kinematic parameter value set is obtained. The latched value set can be used as a reference value set for subsequent kinematic parameter values included in any subsequently acquired value sets received by the controller. Once latched, the latched kinematic parameter values can be used as a reference to the subsequently acquired values such that changes in the kinematic parameters of the system can be observed. In some embodiments, the latch is reset on condition that the compared value is outside of the defined range. In some embodiments, the latch is reset on condition that the compared value is within the defined range. In such embodiments, the controller 4900 places the system in instrument release mode. The controller can additionally or alternatively log the use of the manual input device 4860. In some embodiments, the system can return to normal operation (e.g., teleoperated input following control mode or instrument locked control mode) after the jam on the end effector is cleared by using the manual input device 4860. In such embodiments, after the mode has be changed the algorithm can begin again and the latch is resent. In other embodiments, the system may require that the instrument 4400 be replaced after the jam on the end effector is cleared. In some embodiments, the instrument can be put into a limited teleoperated following control mode if the user attempts to return to teleoperated following control mode after the manual release has been used. In the limited teleoperated following control mode, the system locks certain degrees of freedom (e.g., the ones coupled to the manual release mechanism) while still allowing teleoperation to prevent damage to the manual release mechanism.

In accordance with some embodiments, a variety of instrument types can be installed on the manipulator unit 4200. For example, one instrument 4400 can include clamping end effector tools. Another instrument can include cutting end effector tools. Another instrument can include tissue manipulation end effector tools. Any suitable tool is contemplated herein. The different instrument types can have different range values. As such, the controller 4900 can detect the instrument type and apply the correct range value for the instrument. In some embodiments, the method includes determining if an instrument operatively coupled to the actuator system is one of a first instrument type or a second instrument type different from the first instrument type. In some embodiments, the controller 4900 then commands the actuator system to operate in the instrument release mode only on a condition in which the instrument type is the first instrument type and on the condition in which the compared value is within a defined value range for the first instrument type. A second instrument type can subsequently or alternatively be loaded on the manipulator device. The second instrument type is different from the first instrument type. The controller 4900 then commands the actuator system to operate in the instrument release mode only on a condition in which the compared value is within a defined value range for the second instrument type. In some embodiments, the instrument defined value range is different between the first instrument defined value range and the second instrument defined value range.

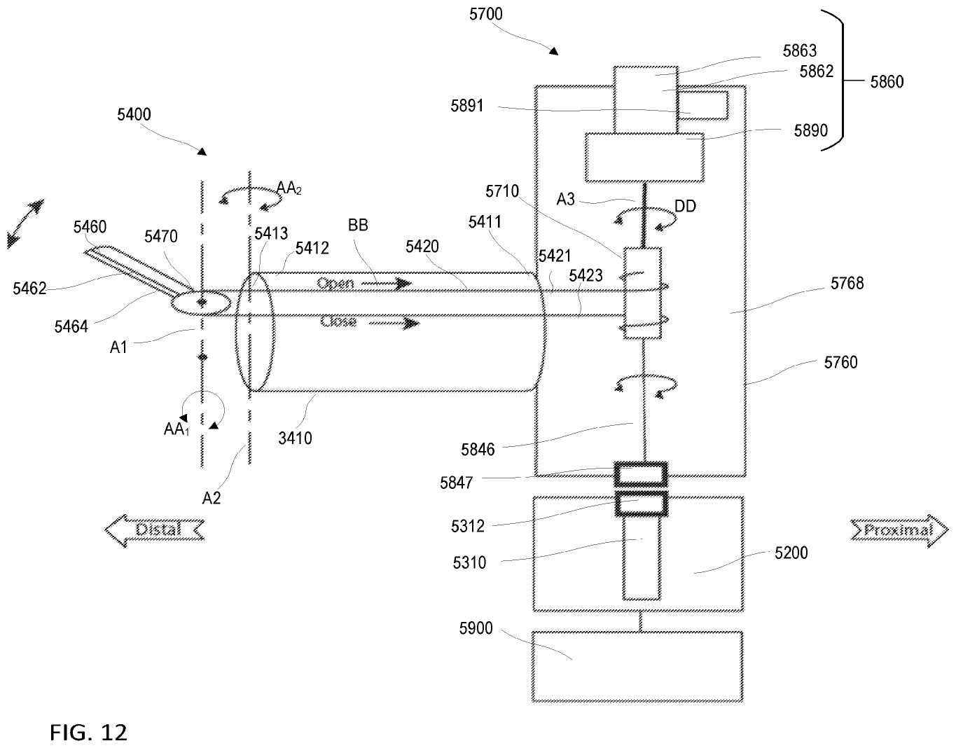

FIG. 12 is a schematic illustration of a portion of a surgical system 5000 according to an embodiment. The surgical system 5000 includes a surgical instrument 5400, a manipulator unit 5200, and a controller 5900. The manipulator unit 5200 (which functions as an actuator system) is in communication with the controller 5900 such that the controller 5900 can send and receive signals from the manipulator unit 5200. For example, the controller 5900 can receive input control signals from the input control unit (such as user control unit 1100 in FIG. 1 or user control unit 2100 in FIG. 5) based on surgeon input (receivable, for example, at the control input function 2908 of controller 2900 in FIG. 5). The controller 5900 then sends control signals to the manipulator unit 5200 (as an example, the device control function 2914 of controller 2900 sends the control signals as illustrated in FIG. 5) which, in turn, drives the surgical instrument 5400. In this manner the manipulator unit 5200 (and any other manipulator units described herein) functions as an actuator system that actuates the surgical instrument 5400. The surgical instrument includes a mechanical structure 5700 having a manual drive mechanism 5860. As discussed in more detail below the manual drive mechanism 5860 drives a powertrain that includes one or more of a tool drive member 5710, input member 5846, and input disk 5847. While shown in FIG. 12 with a single powertrain, multiple power trains can be included (see e.g., FIGS. 6A, 6B, and 14-25B). These powertrain components are also variously referred to as movable components. As shown in FIG. 12, the manual drive mechanism 5860 includes mechanical components to drive the powertrain by external forces from the user by a single actuation of the manual input device 5863. The first powertrain can also be driven by electromechanical drive 5310. Drive disk 5312 can engage with input disk 5847. Due to the engagement between the disks, the manual drive mechanism 5860 is also sufficiently mechanically coupled with electromechanical drive 5310 to drive (or cause some movement of) electromechanical drive 5310. Because of the mechanical coupling between the electromechanical drive 5310, via the powertrain in the instrument 5400, the electromechanical drive 5310 can produce a signal that reflect one or more kinematic parameters of the movable component (e.g., 5710, 5846, 5846, etc.) in the surgical instrument 5400. This allows the controller 5900 to recognize use of the manual drive 5863 and change operation modes to accommodate the use of the manual drive 5863.

As shown in FIG. 12, in some embodiments, the medical device 5400 includes a shaft 5410, a tension member 5420, an end effector 5460, and the mechanical structure 5700. The mechanical structure 5700 functions to receive one or more motor or manual input forces or torques and mechanically transmit the received forces or torques to move the end effector 5460. For example, as described above the manipulator unit 5200 can include one or more electric motors (which function as an electromechanical drive, e.g., the electromechanical drive 5310) to provide an input to the mechanical structure 5700, which in turn transmits the input via the tension member 5420 to control the end effector 5460. Specifically, the mechanical structure includes a chassis 5768, a tool drive member 5710 and a manual drive mechanism 5860. The chassis 5768 provides the structural support for mounting or supporting and aligning the components of the mechanical structure 5700. For example, openings, protrusions, mounting brackets and the like can be defined in or on chassis 5768. In some embodiments, the chassis 5768 can include multiple portions, such as an upper chassis and a lower chassis. In some embodiments, a housing 5760 can optionally enclose at least a portion of the chassis 5768.

The tool drive member 5710 is mounted to the mechanical structure 5700 (e.g., within the housing 5760) via a first tool drive member support member (not shown). For example, the first tool drive member support member can be a mount, shaft, or any other suitable support structure to secure the tool drive member 5710 to the mechanical structure 5700. The tool drive member 5710 includes (or is coupled to) a first input member 5846 and an input disk 5847. The first input member 5846 can be connected to and receive mechanical input from the electromechanical drive 5310. The tool drive member 5710 can be operable to be rotated about an axis A3 in a direction DD, as shown in FIG. 12.

The manual drive mechanism 5860 includes manual input device 5863, a manual drive input member 5862, a sensor 5891, and a manual drive coupling member 5890. The manual drive input member 5862 is mechanically connected to the manual input device 5863. The manual input device 5863 includes a portion that is exposed to the exterior of the medical device 5400. The user can engage the manual input device 5863 and manipulate the manual drive mechanism 5860, thereby manipulating the end effector 5460. The exposed portion of the manual input device 5863 can include any suitable structure for receiving the user's input force. For example, the manual input device 5863 can include a rotatable wheel, a rotatable knob, a push button, a slide, or other suitable mechanical structures that receive the user's input force and allows the manual drive mechanism 5860 to translate the user's input motion to an input on the tool drive members 5710 and thereby manipulate the end effector 5460.

The sensor 5891 of the manual drive mechanism 5860 is configured to detect a kinematic parameter of the manual drive mechanism 5860. For example, the sensor 5891 detects a change in position (e.g., angular orientation, transitional movement, etc.). The sensor can be any suitable device for detecting the kinematic parameter change of the manual drive mechanism 5860. For example, the sensor can be a switch that is triggered by a change in angular orientation of the manual drive mechanism. In other examples, the sensor can be a speed sensor, torque sensor, or similar suitable device.

The manual drive coupling member 5890 is connected to the manual drive input member 5862. The manual drive coupling member 5890 transmits the user's force from the manual drive input member 5862 to at least one tool drive member 5710 (in other embodiments the coupling member can transmit the user's force to a second tool drive member see FIGS. 6A, 6B and 14-25B). In some embodiments, the manual drive coupling member 5890 allows the input force to be transmitted from the manual drive input member 5862 toward the tool drive member 5710, but not from the tool drive member 5710 toward the manual drive input member 5862. Said another way, in some embodiments, the manual drive coupling member 5890 does not allow movement of the tool drive member 5710 to cause movement of the manual drive input member 5862.

The manual drive coupling member 5890 can include any suitable structure or components to perform the functions described herein. For example, in some embodiments, the manual drive coupling member 5890 can include a gear member or set of gears that can be engaged and disengaged from the tool drive member 5710. In other embodiments clutches, tension members, hydraulics, slidable linkages, or any other suitable mechanism for transmitting force from the user to the tool drive members some of which are discussed herein and others that a person of ordinary skill in the art would apply based on the disclosure provided herein.

The tension member 5420 includes a first proximal portion 5421, a second proximal portion 5423 and a distal portion 5422. The first proximal portion 5421 and the second proximal portion 5423 are each coupled to the mechanical structure 5700, and the distal portion 5422 is coupled to the end effector 5460. The shaft 5410 includes a proximal end portion 5411 and a distal end portion 5412 and defines a passageway 5413 that extends lengthwise through the shaft between the proximal and distal end portions. In accordance with various embodiments, the tension member includes any member suitable tension member for transmitting force between the tool drive member 5710 and the end effector. For example, the tension member can include one or more of a cable, band, strap, string, wire, tube, rod, etc. The tool drive member 5710 includes one or more of capstans, winches, spools, or other suitable devices for containing, controlling, taking up, and dispensing the tension member 5420).

As shown in FIG. 12, the tool drive member and the tension member can be mechanically connected in a self-antagonistic drive arrangement. However, it is appreciated that in other embodiments, the tool drive member and the tension member can be mechanically connected in an antagonistic drive arrangement along with an additional tool drive member (compare to FIG. 6A and FIG. 6B). However, for clarity, the self-antagonistic drive is discussed herein.

The end effector 5460 is rotatably coupled to the distal end portion 5412 of the shaft 5410 and includes at least one tool member 5462. The medical device 5400 is configured such that movement of the first proximal portion 5421 and the second proximal portion 5423 of the tension member 5420 produces movement of the tool member 5462 about a first axis of rotation A1 (which functions as the yaw axis; the term yaw is arbitrary), in a direction of arrows AA1. In some embodiments, the medical device 5400 can include a wrist assembly including one or more links that couples the end effector 5460 to the distal end portion 5412 of the shaft 5410. In such an embodiment, movement of the first proximal portion 5421 and the second proximal portion 5423 of the tension member 5420 can also produce movement of the wrist assembly about a second axis of rotation A2 (which functions as the pitch axis) or both movement of the wrist assembly and the end effector 5460. See, for example, U.S. provisional application No. 63/233,904 incorporated by reference above.

The tool member 5462 includes a contact portion 5464 and a drive pulley 5470. The contact portion 5464 is configured to engage or manipulate a target tissue during a surgical procedure. For example, in some embodiments, the contact portion 5464 can include an engagement surface that functions as a gripper, cutter, tissue manipulator, or the like. In this manner, the contact portion 5464 of the tool member 5462 can be actuated to engage or manipulate a target tissue during a surgical procedure. The tool member 5462 (or any of the tool members described herein) can be any suitable medical tool member. Moreover, although only one tool member 5462 is shown, in other embodiments, the medical device 5400 can include two or more moving tool members that cooperatively perform gripping or shearing functions.

As shown in FIG. 12, in a self-antagonistic arrangement, the tension member 5420 is routed from the mechanical structure 5700 to the end effector 5460 and then back to mechanical structure 5700, and each individual end of the tension member 5420 is coupled to the tool drive member 5710 (in an antagonistic arrangement each end would couple to different tool drive members) of the mechanical structure 5700.

The two ends of the tension member 5420 that are associated with opposing directions of a single degree of freedom of the tool member 5462 are connected to the same tool drive member 5710. This arrangement allows for singular control of the movement of (e.g., pulling in or paying out) each of the ends of the tension member 5420 by the same tool drive member. The mechanical structure 5700 produces movement of the tension member 5420, which operates to produce the desired articulation movements (pitch, yaw, or grip) at the end effector 5460. Accordingly, as described herein, the mechanical structure 5700 includes components and controls to move the first proximal portion 5421 of the tension member 5420 via the tool drive member 5710 in a first direction (e.g., a proximal direction) and a second opposite direction (e.g., a distal direction).

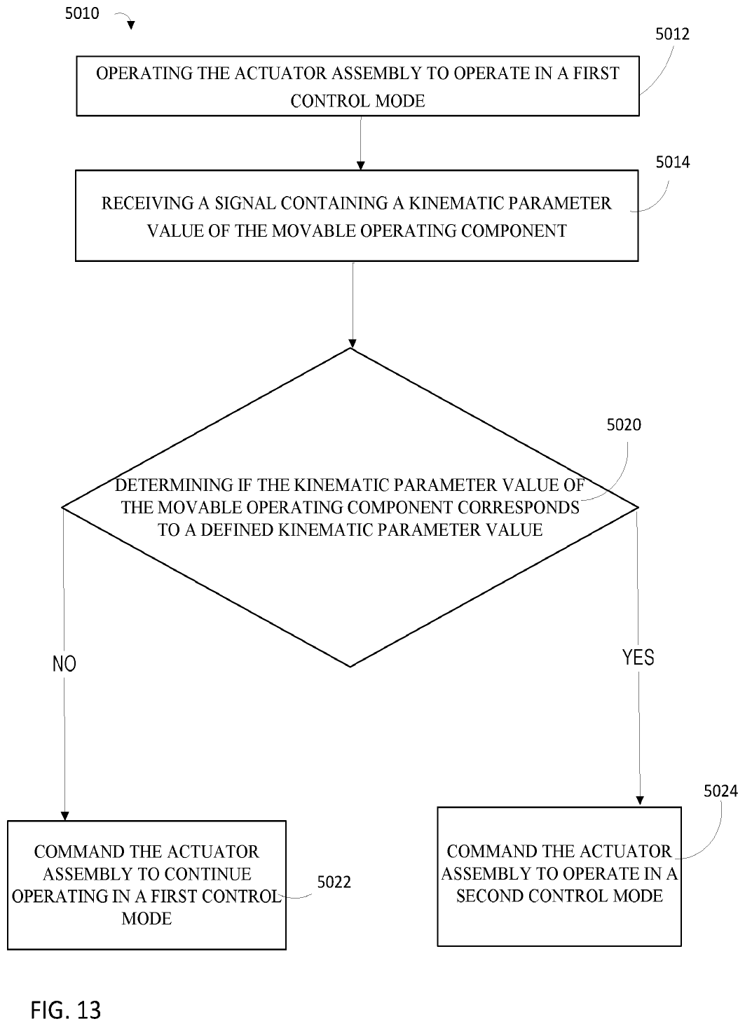

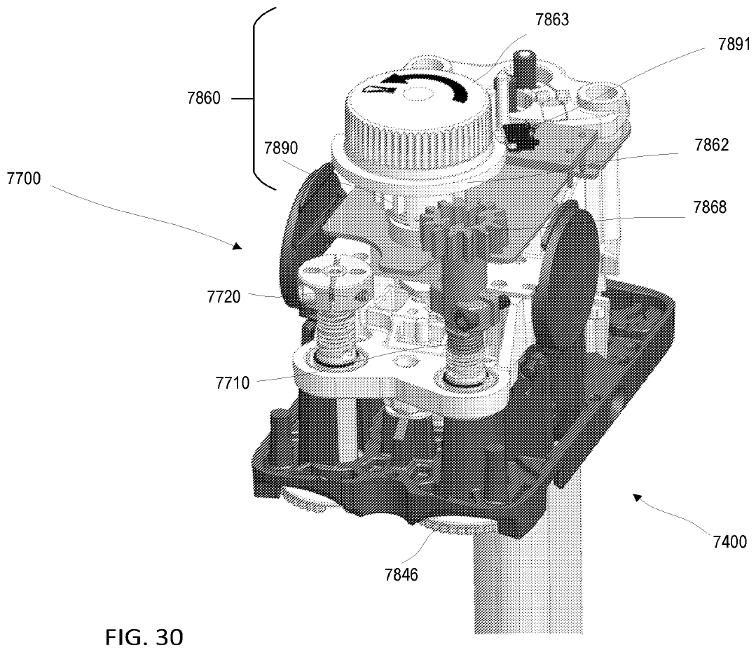

FIG. 13 is a flow chart of a control method 5010 for a surgical system according to an embodiment. The method 5010 can, in an embodiment, be performed via the surgical system as described with reference to any of the systems shown in FIGS. 1-5 and 12, or any other systems herein. However, it should be appreciated that in various embodiments, aspects of the method 5010 may be accomplished via additional embodiments of the surgical system or components thereof, such as instrument 6400 (shown in FIG. 30). The method 5010 may be implemented, at least in part, via the controller 5900 shown in FIG. 12 (similarly described in FIG. 5 with reference to controller 2900). Instruction for operating the controller and the execution of the methods and processes 5010 can be stored in associated memory device(s) which are configured to perform the variety of computer implemented functions (e.g., performing the methods, steps, calculations and the like and storing relevant data as disclosed herein.

As depicted at 5012, the controller 5900 operates the actuator system (e.g., the manipulator unit 5200) in a variety of modes. These modes can include a teleoperated input following control mode, a limited teleoperated input following control mode, an instrument locked control mode, an instrument release control mode, along with other suitable operational control modes. References are made herein to first control modes and second control modes. In accordance with the embodiments, discussed herein, the system can switch between these control modes according to various algorithmic constraints. In some embodiments, the first control mode can be a locked control mode. In some embodiments, the first control mode can be a teleoperated input following control mode. In some embodiments, the first control mode can be a limited teleoperated input following control mode. In some embodiments, the second control mode is an instrument release mode.

In some embodiments, instrument locked control mode can be instituted in response to the system encountering an error. The error can be due to an unresponsive end effector due to a jam or other unexpected restriction in movement. In some embodiments, the instrument locked control mode can be instituted in response to the surgeon actively engaging the instrument locked control mode. The instrument locked control mode can lock inner and outer degrees of freedom of the various joints of the surgical instrument to limit unintended movement.

In some embodiments the teleoperated input following control mode can include various different modes of operation that allow the surgeon to guide the instrument 5400 from the control unit (e.g., control unit 2100 illustrated in FIG. 5 and control unit 1100 illustrated in FIGS. 1 and 2). At least one of these various different modes of operation provides control of the instrument 5400 based on input by the surgeon. Input at the control unit allows the surgeon to guide the instrument 5400. Under standard or typical conditions of the surgical system, the surgeon is allowed to provide input to perform surgery on the patient with the instrument 5400. The teleoperated input following control mode is an operation mode of the surgical system 5000 that contrasts with, for example, modes in which the surgical system 5000 is operating under an error state in which the surgical system 5000 is not performing as directed by the surgeon from the control unit. In some embodiments, the surgical system 5000 can include a step to operate the system in the teleoperated input following control mode prior to operating the system in the instrument locked mode.

In some embodiments the limited teleoperated input following control mode can include various different modes of operation that allow the surgeon to guide the instrument 5400 from the control unit (e.g., control unit 2100 illustrated in FIG. 5 and control unit 1100 illustrated in FIGS. 1 and 2). This movement can be limited by reducing torque or locking certain joints to restrict their degrees of freedom (dofs.). In some embodiments, the instrument can be operated in the limited teleoperated input following control mode after the system has operated in the instrument release mode. This can help protect the manual input mechanism from damage by locking dofs. that the manual input mechanism controls.

As depicted at 5012, the controller 5900 initial command to operate in first control mode. If the initial command has already been processed, the system can continue to operate in the first control mode (e.g., instrument locked mode or teleoperated input following control mode) or in some embodiments, it can operate in an alternative control mode (e.g., limited teleoperated input following control mode). At 5012, the controller 5900 commands the actuator system to operate in the first control mode, allowing the surgeon to proceed with the surgical procedure.

In accordance with some embodiments, the surgical system 5000 monitors the functionality and activity of one or more components of the surgical system 5000. For example, the surgical system 5000 can monitor kinematic parameters of components in the instrument 5400. These kinematic parameters can be monitored by receiving signals from a sensor 5891. As depicted at 5014, the controller 5900 receives a signal containing a kinematic parameter value. The kinematic parameter value can be related to or indicative of a kinematic parameter of the manual input mechanism 5860. For example, as described below, the kinematic parameter can be an angular orientation of the manual input mechanism 5860. In such an example, the kinematic parameter value can be a signal that corresponds to a change in angular orientation from a starting position. In some embodiments, individual values from the signal can be utilized. For example, the values can be digital. In one embodiment, the presence of any signal can indicate the manual input mechanism 5860 is active and the absence of any signal can indicate manual input mechanism is inactive. In another embodiment, the absence of any signal can indicate the manual input mechanism 5860 is active and the presence of any signal can indicate manual input mechanism is inactive.

As depicted at 5020, the controller 5900 determines if the kinematic parameter value indicates use of the manual input mechanism 5860. Depending on the value received from the sensor 5891, the controller 5900 can execute different instructions. For example, in response to a condition in which the value non-usage of the manual input mechanism 5860, as depicted at 5022, the control system continues to operate the actuator system in a first control mode. In response to a condition in which the value indicates usage of the manual input mechanism 5860, as depicted at 5024, the control system operates the actuator system in an instrument release mode.

In some embodiments, the kinematic parameter value can be received at and processed by the kinematic parameter signal function within the controller 5900. An example of such function and controller is the kinematic parameter signal function 2910 of controller 2900 as illustrated in FIG. 5.

In accordance with some embodiments, the instrument release mode includes surgical system 5000 operational modes that limit the ability of the user input unit to drive the instrument 5400. In particular, the controller 5900 would limit the ability of electromechanical actuator 5310, along with any other electromechanical actuators, to overpower the use of the manual input mechanism 5860. Generally, the first actuator powertrain is operable to drive the first movable operating component of the instrument. However, in accordance with some embodiments, in instrument release mode, the actuation applied by the electromechanical actuator to the first powertrain is limited to merely applying a dampening torque in the first movable operating component. Similarly, the actuation applied by any additional electromechanical actuator to any additional powertrain is limited to merely applying a dampening torque in the additional movable operating components of the instrument. The first dampening torque is less than a torque applied to the first movable component by movement of the manual control input. Similarly, the second dampening torque is less than a torque applied to the second movable component by movement of the manual control input. In accordance with some embodiments, the dampening torque is the minimal sufficient torque to hold the surgical instrument in place and less than the torque applied by the manual input device.

In accordance with some embodiments, a latched kinematic parameter value is obtained. The latched value can be used as a reference value for a subsequent kinematic parameter value received by the controller. Once latched, the latched kinematic parameter value can be used as a reference to the subsequently acquired value such that changes in the kinematic parameters of the system can be observed. In some embodiments, the latch is reset on condition that the value is indicates non-usage (e.g., does not match or is outside of a defined range). In some embodiments, the latch is reset on condition that the value indicates usage (e.g., matches or is within a defined range. In such embodiments, the controller 5900 places the system in instrument release mode. The controller can additionally or alternatively log the use of the manual input device 5860. In some embodiments, the system can return to normal operation (e.g., teleoperated input following mode or instrument locked control mode) after the jam on the end effector is cleared by using the manual input device 5860. In such embodiments, the latch is resent in response to returning to the first control mode. In other embodiments, the system may require that the instrument 5400 be replaced after the jam on the end effector is cleared. In some embodiments, the instrument can be put into a limited teleoperated following control mode if the user attempts to return to teleoperated following control mode after the manual release has been used. In the limited teleoperated following control mode, the system locks certain degrees of freedom (e.g., the ones coupled to the manual release mechanism) while still allowing teleoperation to prevent damage to the manual release mechanism.

In accordance with some embodiments, a variety of instrument types can be installed on the manipulator unit 5200. For example, one instrument 5400 can include clamping end effector tools. Another instrument can include cutting end effector tools. Another instrument can include tissue manipulation end effector tools. Any suitable tool is contemplated herein. The different instrument types can have different range values. As such, the controller 5900 can detect the instrument type and apply the correct value range for the instrument. In some embodiments, the method includes determining if an instrument operatively coupled to the actuator system is one of a first instrument type or a second instrument type different from the first instrument type. In some embodiments, the controller 5900 then commands the actuator system to operate in the instrument release mode only on a condition in which the instrument type is the first instrument type and on the condition in which the compared value is within a defined value range for the first instrument type. A second instrument type can subsequently or alternatively be loaded on the manipulator device. The second instrument type is different from the first instrument type. The controller 5900 then commands the actuator system to operate in the instrument release mode only on a condition in which the compared value is within a defined value range for the second instrument type. In some embodiments, the instrument defined value range is different between the first instrument defined value range and the second instrument defined value range.

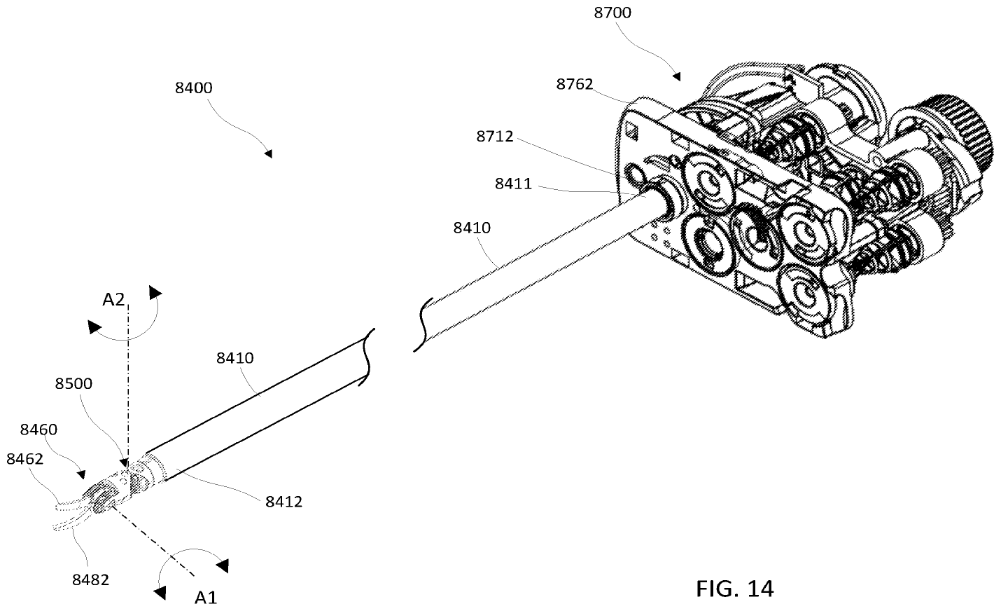

FIGS. 14-25B are various views of a medical instrument 8400, according to an embodiment. In some embodiments, the medical device 8400 or any of the components therein are optionally parts of a surgical system (e.g., system 1000) that performs surgical procedures, and which can include a manipulator unit, a series of kinematic linkages, a series of cannulas, or the like. As shown in FIG. 14, the medical device 8400 includes a proximal mechanical structure 8700, a shaft 8410, a distal wrist assembly, and a distal end effector 8460. In accordance with various embodiments the medical instrument 8400 includes a manual input mechanism 8860 suitable to release an end effector (e.g., 8462). The medical instrument manual input mechanism is also coupled to the mechanical structure 8700 so as to allow indication of its use through the combine actuation of two tool drive members (e.g., capstan 8710 and 8720 discussed below). The description provided with respect to FIGS. 14-25B is one embodiment of a medical instrument 8400 that indicates use of a manual input mechanism 8860 to the controller of the instrument.

The shaft 8410 can be any suitable elongated shaft that couples the wrist assembly to the mechanical structure 8700. Specifically, the shaft 8410 includes a proximal end 8411 that is coupled to the mechanical structure 8700, and a distal end 8412 that is coupled to the wrist assembly (e.g., a proximal link of the wrist assembly).

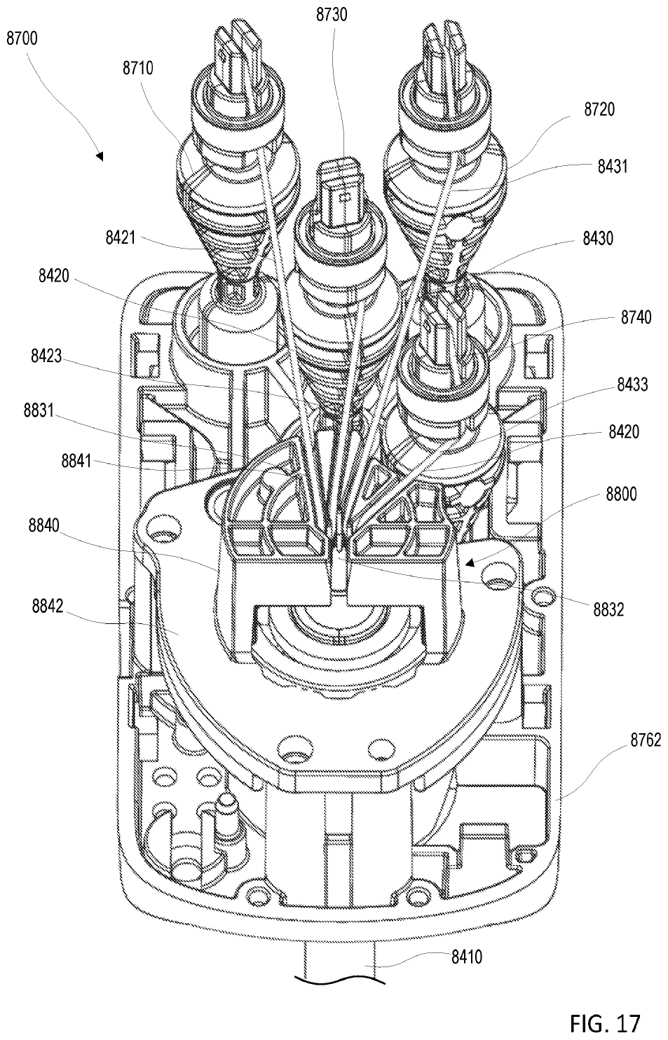

As shown in FIG. 17, the medical device 8400 also includes a first cable 8420 and a second cable 8430 that couple the proximal mechanical structure 8700 to the distal wrist assembly and end effector 8460. The medical device 8400 is configured such that movement of the first cable 8420 and second cable 8430 produces rotation of the end effector 8460 about a first axis of rotation A1 (which functions as the yaw axis; the term yaw is arbitrary) of the wrist assembly about a second axis of rotation A2 (which functions as the pitch axis; the term pitch is arbitrary), a cutting rotation of the tool members of the end effector 8460 about the first axis of rotation, or any combination of these movements. Additional examples and disclosures of the actuation of the end effector with relevant axes, e.g., first axis A1 and second axis A2, are further disclosed in U.S. provisional application No. 63/233,904 entitled “Surgical Instrument Cable Control and Routing Structures” filed on Aug. 17, 2021, which is incorporated herein by reference in its entirety. Changing the pitch or yaw of the medical device 8400 can be performed by manipulating the cables in a similar manner as that described above for the instruments 2400, 3400, 4400, and 5400. Thus, the specific movement of each of the cables to accomplish the desired motion is not described below.

The first cable 8420 includes a first proximal portion 8421, a second proximal portion 8423, and a distal portion (not shown). The second cable 8430 includes a first proximal portion 8431, a second proximal portion 8433, and a distal portion (not shown). As described in more detail below, the first proximal portion 8421 is coupled to a first capstan 8710 and the second proximal portion 8423 is coupled to a third capstan 8730. The distal portion of the first cable 8420 is coupled to a first tool member 8462. Thus, movement of the first capstan 8710 and the third capstan 8730 can move the proximal end portions of the first cable 8420 to move the first tool member 8462. The first proximal portion 8431 is coupled to a second capstan 8720) and the second proximal portion 8423 is coupled to a fourth capstan 8740. The distal portion of the second cable 8430 is coupled to a second tool member 8482. Thus, movement of the second capstan 8720 and the fourth capstan 8740 can move the proximal end portions of the second cable 8430 to move the second tool member 8482.

The end effector 8460 can be operatively coupled to the mechanical structure 8700 such that the tool members 8462 and 8482 rotate about the first axis of rotation A1. For example, a drive pulley (not shown) of the first tool member 8462 is coupled to the distal end of the first cable 8420 such that a tension force exerted by the first cable 8420 produces a rotation torque about the first axis A1. Similarly, a drive pulley (not shown) of the second tool member 8482 is coupled to the distal end of the second cable such that a tension force exerted by the second cable produces a rotation torque about the first rotation axis A1. In this manner, the tool member 8462 and the tool member 8482 can be actuated to engage or manipulate a target tissue during a surgical procedure.

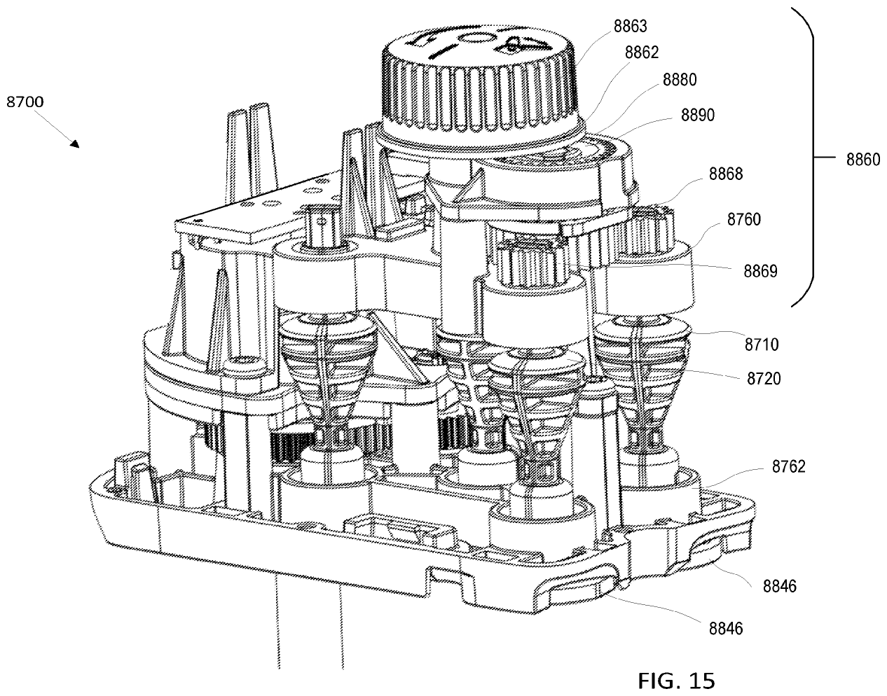

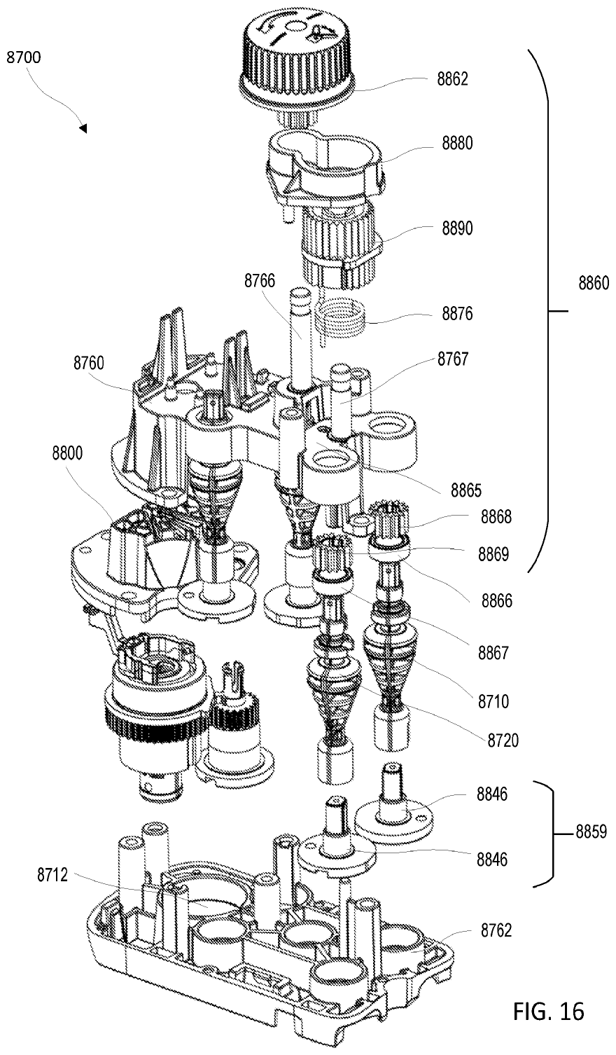

For actuation of the end effector 8460, the proximal mechanical structure 8700 includes motor drive structure 8859 and a manual drive mechanism 8860 as shown in FIGS. 15 and 16. Additionally, the mechanical structure 8700, as shown in FIGS. 15-18, includes an upper chassis 8760, a lower chassis 8762, the first capstan 8710, the second capstan 8720, the third capstan 8730, the fourth capstan 8740, and a cable guide 8800. The manual drive mechanism 8860 includes a manual drive input member 8862, a manual drive coupling member 8890, a first capstan gear 8868 (which functions as a first manual drive input), and a second capstan gear 8869 (which functions as a second manual drive input). Additionally, the manual drive mechanism 8860 can also include a biasing member 8876 and bracket 8880. Both the motor drive structure 8859 and the manual drive mechanism 8860 are connected to and drive the first capstan 8710 and the second capstan 8720. As discussed in more detail below; the capstans 8710, 8720, 8730, 8740 are connected to and actuate, via the motor drive structure 8859 or the manual drive mechanism 8860, the first cable 8420 and the second cable 8430.

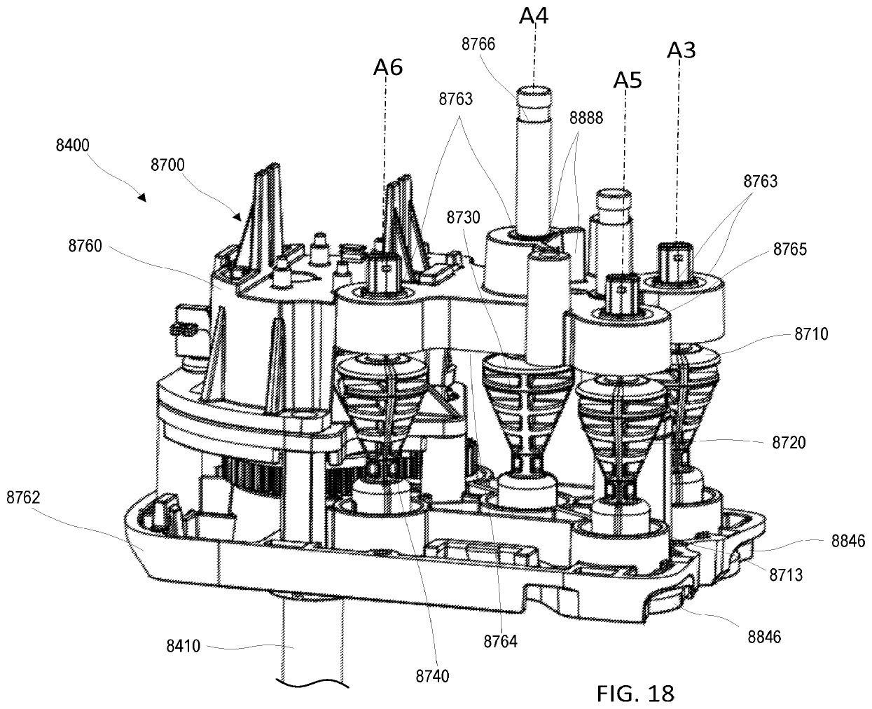

In some embodiments, the upper chassis 8760 and the lower chassis 8762 may partially enclose or fully enclose other components of mechanical structure 8700. In some embodiments, a housing cover (not shown) encloses the mechanical structure 8700, including the upper chassis 8760 and the lower chassis 8762. The lower chassis 8762 and the upper chassis 8760 provide structural support for mounting and aligning components in the mechanical structure 8700. For example, the lower chassis 8762 includes a shaft opening 8712 (see FIGS. 14 and 16), within which the proximal end 8411 of the shaft 8410 is mounted. The lower chassis 8762 further includes one or more bearing surfaces or openings 8713, within which the capstans (e.g., the first capstan 8710 and the second capstan 8720) are mounted and rotatably supported. The upper chassis 8760 also includes openings 8763 in a bottom 8764, within which an upper portion of the capstans are mounted as described in more detail below. The openings 8763 of the upper chassis 8760 are axially aligned with the openings 8713 of the lower chassis 8762 to support the capstans. As shown in FIGS. 16 and 18, the upper chassis 8760 includes a first spindle 8766 and a second spindle 8767. The first spindle 8766 extends from the structure that includes the end stops 8888 (described in more detail below) and receives the manual drive input member 8862. Thus, in use, when the user rotates the manual drive input member 8862, the manual drive input gear 8864 rotates about the first spindle 8766. The second spindle 8767 extends from the top surface 8865 and receives the manual drive coupling member 8890. Thus, in use when the manual drive input gear 8864 rotates about the first spindle 8766, its engagement with the manual drive coupling member 8890 (described below) causes the manual drive coupling member 8890 to rotate about the second spindle 8767.

In addition to providing mounting support for the internal components of the mechanical structure 8700, the lower chassis 8762 can include external features (e.g., recesses, clips, etc.) that interface with a docking port of a drive device (not shown). The drive device can be, for example, a handheld system or a computer-assisted teleoperated system that can receive the medical device 8400 and manipulate the medical device 8400 to perform various surgical operations. The drive device can include one or more motors to drive capstans of the mechanical structure 8700. In other embodiments, the drive device can be an assembly that can receive and manipulate the medical device 8400 to perform various operations.

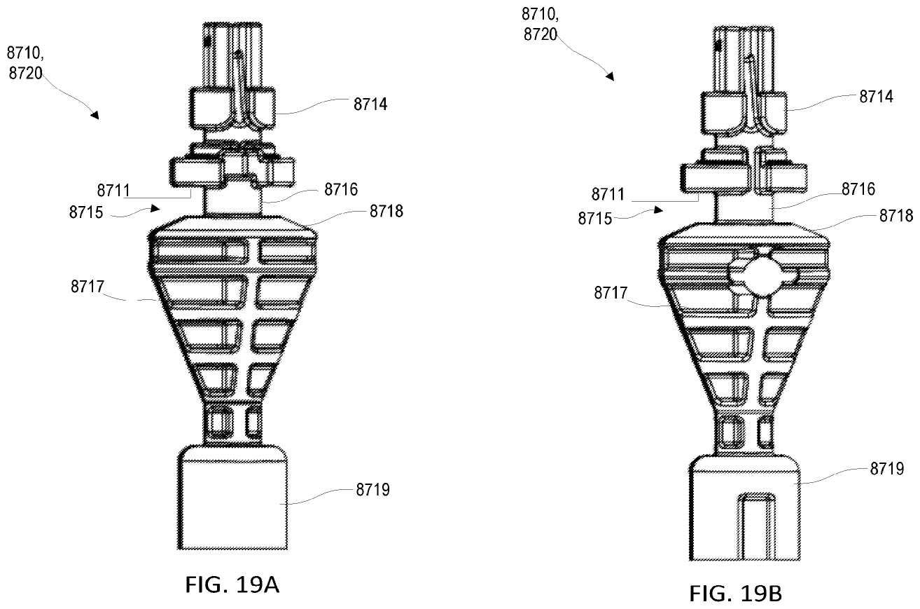

As shown in FIGS. 19A and 19B, the first capstan 8710 includes an upper portion 8714, a lower portion 8717, and a spool 8715 between the upper portion 8714 and the lower portion 8717. The upper portion 8714 functions as an anchor portion to secure the first cable 8420 to the capstan 8710. In some embodiments, the upper portion 8714 can include a specific configuration to allow for a cable to be coupled to the capstan without the use of external mechanisms (e.g., crimp joints, adhesive, knots) to maintain the coupling of the cable to the capstan 8710. Such configuration can include, for example, grooves and recesses within which the cable can be wrapped, as shown and described in U.S. provisional application No. 63/233,904 entitled “Surgical Instrument Cable Control and Routing Structures” filed on Aug. 17, 2021, which is incorporated herein by reference in its entirety. In other embodiments, however, the upper portion can include recesses or channels that receive a crimp or know to secure the cable therein. The spool 8715 includes a cable wrap surface 8716 (which functions as a drive surface) and a side wall 8718. The first cable 8420 is coupled to the first capstan 8710 such that a proximal end portion of the first cable 8420 wraps about the cable wrap surface 8716 of the first capstan 8710. While other capstans are shown, they are not discussed in detail herein.

The lower portion 8717 of the first capstan 8710 is supported by the lower chassis 8762, and the upper portion 8714 of the first capstan 8710 is supported within the opening 8763 defined in the bottom 8764 of the upper chassis 8760 (see, e.g., FIG. 18). In some embodiments, the bottom 8764 of the upper chassis 8760 has a continuous planar surface in which the openings 8763 are defined. In some embodiments, the bottom 8764 of the upper chassis has portions with surfaces in which the openings 8763 are defined, such as by the bottom of a support web structure of bracing material in the upper chassis. In some embodiments, a bottom 8711 of the upper portion 8714 (see, e.g., FIGS. 19A and 19B) of the first capstan 8710 is within the opening 8763 such that it is between the bottom 8764 of the upper chassis 8760 and a top surface 8765 of the upper chassis 8760. In other words, the entire upper portion 8714 of the first capstan 8710 is within the opening 8763. In some embodiments, the bottom 8711 of the upper portion 8714 of the first capstan 8710 is positioned flush with the bottom 8764 of the upper chassis 8760. The side wall 8718 of the spool 8715 slopes away from the bottom 8764 of the upper chassis 8760. The second capstan 8720, along with other capstans, can be structured the same as the first capstan 8710 and can be supported by the lower chassis 8762 and the upper chassis 8760 in the same manner, and are therefore not described in detail here.

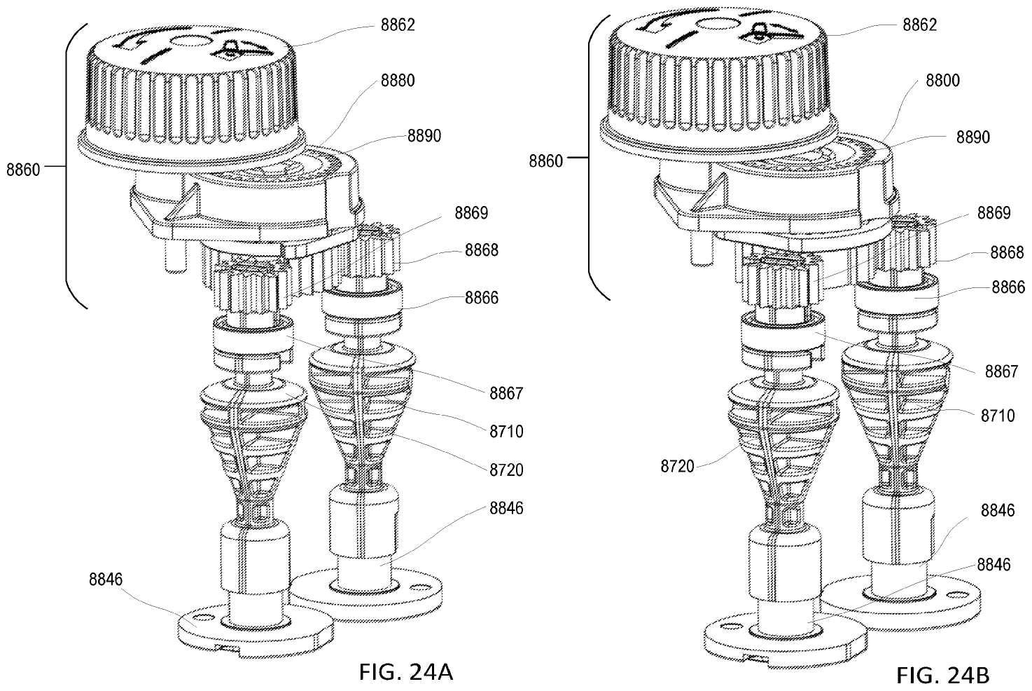

As described above, the upper portion 8714 of each of the capstans 8710, 8720 is rotatably supported within a corresponding opening 8763 (see FIG. 18) of the upper chassis 8760. More specifically, bearings 8866, 8867 are coupled to the upper portion 8714 of the capstans, and the bearings 8866, 8867 are supported within the opening 8763. The bearings 8866, 8867 can be, for example, a rolling-element bearing, such as a ball or needle bearing. As shown in FIGS. 24A and 24B, the upper portion 8714 also provides a protrusion about which the first capstan gear 8868 is coupled. Similarly, the upper portion of the second capstan 8720 provides a protrusion about which the second capstan gear 8869 is coupled.

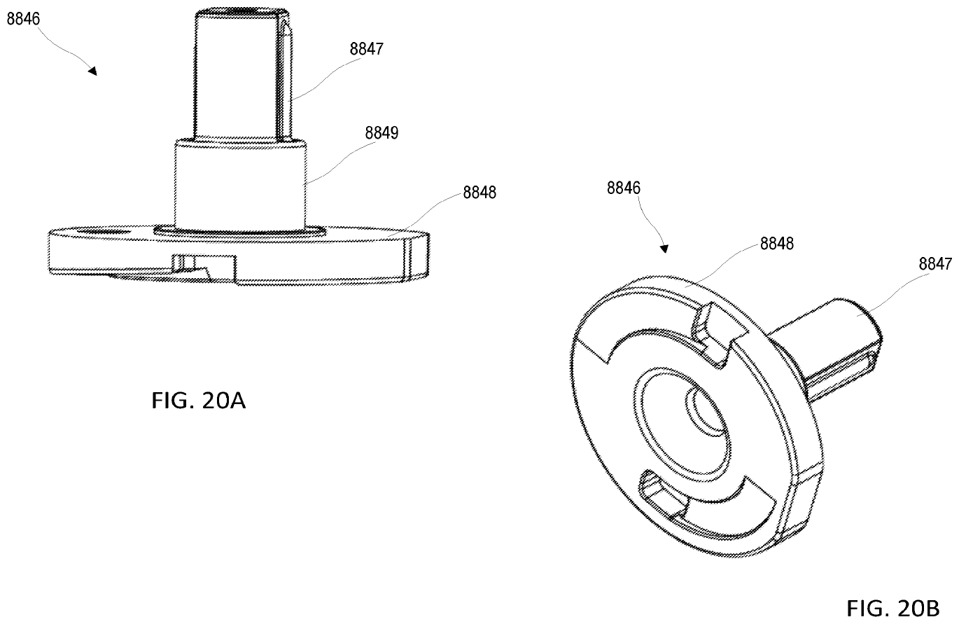

In addition, in this embodiment, the lower portion 8717 of each of the capstans 8710, 8720 is supported by the lower chassis 8762 via bearings. In some embodiments, the drive discs 8846 can include a bearing surface 8849 that interfaces with journal bearings (not shown) within the lower chassis 8762. As shown in FIGS. 20A and 20B, the drive discs 8846 include a neck 8847, a coupling portion 8848, and a bearing surface 8849. The neck 8847 is received within an opening (not shown) in the bottom end portion 8719 of the capstans 8710, 8720. The coupling portion 8848 can be coupled to the lower chassis 8762, for example, within the openings 8713. The end surface of the drive portion 8848 is exposed from under the mechanical structure 8700 and can be mated with a corresponding drive disc in a manipulator. Thus, motors can be operationally coupled to rotate the capstans via the drive discs 8846. The bearing surface 8849 interfaces with a journal bearing pressed within the lower chassis 8762. Any suitable drive disk can be used with the various embodiments disclosed herein. For example, U.S. Pat. No. 10,247,911 entitled “Instrument Sterile Adapter Drive Features”, issued on Apr. 30, 2021, which is hereby incorporated by reference in its entirety, discloses additional systems and features of drive disks that can be used with the embodiments herein.

Each of capstans 8710, 8720, 8730, 8740 can be driven by one or more corresponding motors (not shown) in the drive device (e.g., the manipulator unit 1200) via the motor drive structure 8859 (which includes the drive discs). For example, as shown in FIG. 18, the first capstan 8710 can be driven to rotate about a first capstan axis A3, the second capstan 8720 can be driven to rotate about a second capstan axis A5, the third capstan 8730 can be driven to rotate about a third capstan axis A4, and the fourth capstan 8740 can be driven to rotate about a fourth capstan axis A6.

As shown in FIG. 17, the first proximal portion 8421 of the first cable 8420 is coupled to the first capstan 8710 and extends to a cable guide 8800 within the mechanical structure 8700, where it is rerouted through an interior passageway of the shaft 8410 (not shown in FIG. 17) and extends to the wrist assembly 8500 (not shown in FIG. 17), and to the end effector 8460 (not shown in FIG. 17). A distal portion of the first cable 8420 is coupled to the end effector 8460 (i.e., the first tool member 8462), and then the first cable 8420 extends proximally back through the interior passageway of the shaft 8410, proximally back through the cable guide 8800 and to the third capstan 8730, where a second proximal portion 8423 of the first cable 8420 is coupled to the third capstan 8730. Similarly, the second cable 8430 is also routed between the mechanical structure 8700 and the end effector 8460. More specifically, the proximal end 8431 of the second cable 8430 is coupled to the third capstan 8730 and extends to the cable guide 8800, where it is rerouted through the interior passageway of the shaft 8410 and extends to the wrist assembly 8500 and to the end effector 8460. A distal portion of the second cable 8430 is coupled to the end effector 8460 (i.e., the second tool member 8482), and then the second cable 8430 extends back through the interior passageway of the shaft 8410, through the cable guide 8800, and to the fourth capstan 8740, where the second proximal portion 8433 of the second cable 8430 is coupled to the fourth capstan 8740. Thus, the two proximal end portions of the cable 8420 are coupled to and actuated by two separate capstans (capstans 8710 and 8730) of the mechanical structure 8700. Likewise, the two proximal end portions of the second cable 8430 are coupled to and actuated by two separate capstans (capstans 8720 and 8740)

More specifically, the two ends of the first cable 8420 that are associated with opposing directions of a single degree of freedom are connected to two independent drive capstans 8710 and 8730, and the two ends of the second cable 8430 that are associated with opposing directions of a single degree of freedom are connected to two independent drive capstans 8720 and 8740. This arrangement, which is generally referred to as an antagonist drive system, allows for independent control of the movement of (e.g., pulling in or paying out) each of the ends of the cables. The mechanical structure 8700 produces movement of the first cable 8420 and the second cable 8430, which operates to produce the desired articulation movements (pitch, yaw, cutting or gripping) at the end effector 8460. Accordingly, as described herein, the mechanical structure 8700 includes components and controls to move a first portion 8421 of the first cable 8420 via the first capstan 8710 in a first direction (e.g., a proximal direction) and to move a second portion 8423 of the first cable 8420 via the third capstan 8730 in a second opposite direction (e.g., a distal direction). The mechanical structure 8700 can also move both the first portion 8421 of the first cable 8420 and the second portion 8423 of the first cable 8420 in the same direction. The mechanical structure 8700 also includes components and controls to move a first portion 8431 of the second cable 8430 via the second capstan 8720 in a first direction (e.g., a proximal direction) and to move a second portion 8433 of the second cable via the fourth capstan 8740 in a second opposite direction (e.g., a distal direction). The mechanical structure 8700 can also move both the first portion of the second cable and the second portion of the second cable in the same direction. In this manner, the mechanical structure 8700 can maintain the desired tension within the cables to produce the desired movements at the end effector 8460.

As shown in FIG. 17, the cable guide 8800 includes an upper portion 8840 and a lower portion 8842. The lower portion 8842 is mounted to a component within the mechanical structure 8700, such as the lower chassis 8762. The upper portion 8840 includes multiple guide grooves 8831 on a top guide surface 8841. The guide grooves 8831 extend along the top guide surface 8841 to openings 8832 that are defined in the top surface 8841. As shown in FIG. 17, the cables 8420, 8430 are routed along the top surface 8841 within the guide grooves 8831 and through the openings 8832 to be routed to the interior passageway of the shaft 8410.

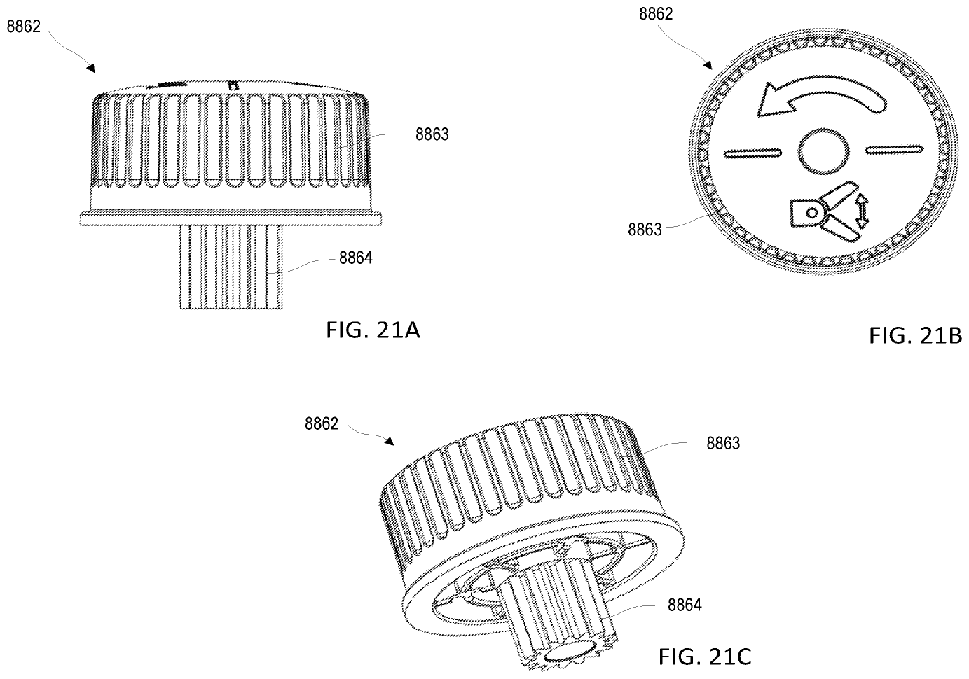

The manual drive mechanism 8860 includes a manual drive input member 8862, a manual drive coupling member 8890, the first capstan gear 8868, the second capstan gear 8869, a biasing member 8876 and a support bracket 8880. As shown in FIGS. 21A-C, the manual drive input member 8862 includes a manual input device 8863 and a manual drive input gear 8864. The manual input device 8863 includes a surface that is exposed to the exterior of the medical device 8400. The user can engage the manual input device 8863 and manipulate the manual drive mechanism 8860, thereby manipulating the end effector 8460. The exposed portion of the manual input device 8863 can include knurls, ridges, or other traction features on the surfaces suitable for the user to apply a torque to the manual drive input member 8862 via the manual input device 8863. As shown in FIGS. 21A-C, the manual input device 8863 includes a rotatable knob that receives the user's input force and allows the manual drive mechanism 8860 to convert the user's input force to a torque on the first capstan 8710 and the second capstan 8720 and thereby manipulate the end effector 8460 via cables 8420 and 8430. Specifically, as described herein, the input force can be converted by the manual drive mechanism to open the jaws of the end effector in the event of a fault, loss of power, or other instance where manual control is desired. As shown in FIG. 21B, the manual input device 8863 also includes indicia (i.e., the arrow and the depiction of jaws opening) to guide the user in the operation of the manual drive mechanism 8860. The manual drive input gear 8864 is connected to and receives any torque applied to the manual input device 8863. The manual drive input gear 8864 includes gear teeth suitable to engage with an adjacent gear (specifically, the manual-drive-side coupling gear 8872).

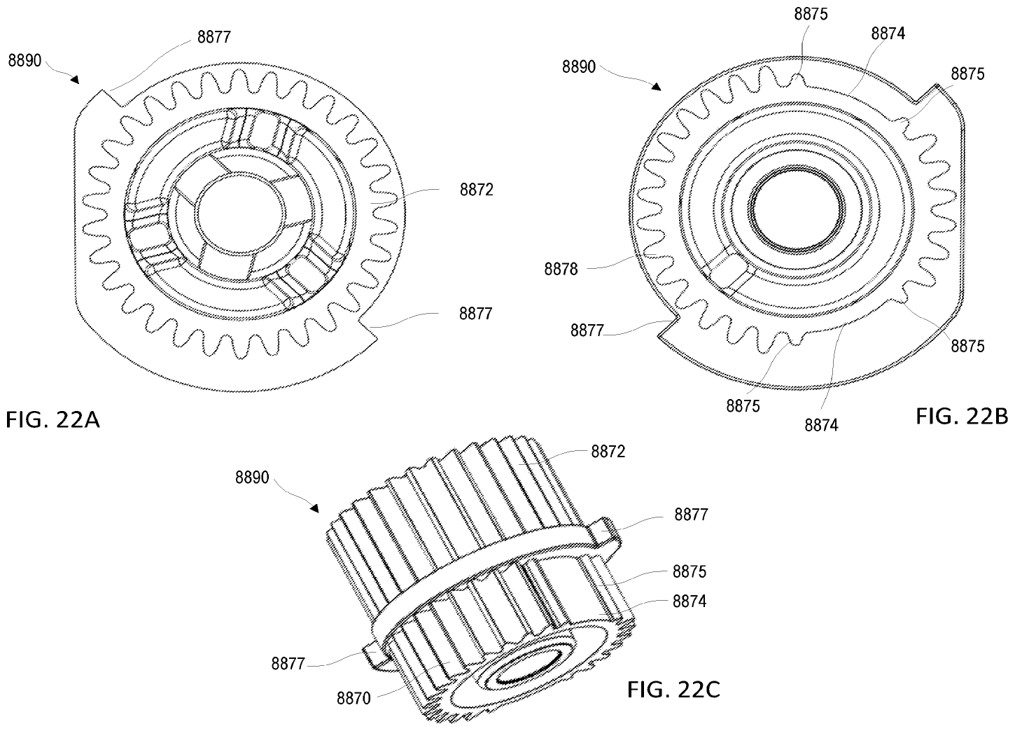

The manual drive coupling member 8890 allows for selective engageability between the manual drive input member 8862 and the capstans 8710, 8720. As shown in FIGS. 22A-C, the manual drive coupling member 8890 includes a tool-drive-side coupling gear 8870, a manual-drive-side coupling gear 8872, and a central portion therebetween that includes two end stops 8877. The tool-drive-side coupling gear 8870 is a gear having teeth suitable to engage the first capstan gear 8868 and the second capstan gear 8869. The tool-drive-side coupling gear 8870 includes two non-engagement portions 8874, two engagement portions 8878, and a gear tooth gradient portion 8875. The non-engagement portions 8874 are portions of the tool-drive-side coupling gear 8870 without engagement teeth. Thus, when the non-engagement portions 8874 of the tool-drive-side coupling gear 8870 is positioned adjacent to (i.e., aligned with) the teeth of the first capstan gear 8868 or the second capstan gear 8869, no torque is transferred between the gears. The engagement portions 8878 are portions of the tool-drive-side coupling gear 8870 with engagement teeth. Thus, when the engagement portions 8878 of the tool-drive-side coupling gear 8870 are positioned adjacent to (i.e., meshed with) the teeth of the first capstan gear 8868 or the second capstan gear 8869, torque is transferred between the gears. The gear tooth gradient portions 8875 is the portion of the gear between the non-engagement portion 8874 and the engagement portion 8878. In this section of the gear, the teeth are smaller than the teeth in the engagement portion 8878 and configured to form a lead-in, allowing for gear engagement as the tool-drive-side coupling gear 8870 transitions from the non-engagement portion 8874 to the engagement portion 8878.

The manual-drive-side coupling gear 8872 is a gear positioned along the same axis as the tool-drive-side coupling gear 8870. The manual-drive-side coupling gear 8872 includes engagement members (e.g., teeth) suitable to engage the manual drive input gear 8864. While FIGS. 22A-C show the tool-drive-side coupling gear 8870 as having the non-engagement portions 8874, in some embodiments the manual-drive-side coupling gear 8872 could alternatively include one or more non-engagement portions 8874. The end stops 8877 are configured to limit the range of rotation of the manual drive coupling member 8890. As shown in FIGS. 22A-C, the manual drive coupling member 8890 includes opposing end stops 8877. Each end stop 8877 can include a contact surface suitable to engage a separate structure (e.g., the end stop 8888 of the upper chassis 8760), thereby limiting the range of rotation of the manual drive coupling member 8890.

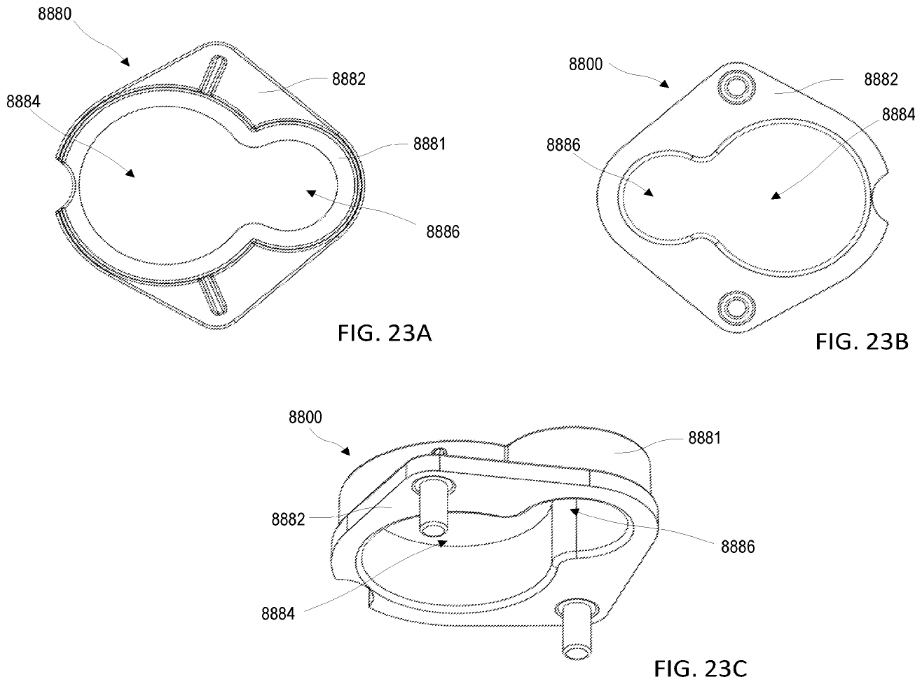

As shown in FIGS. 23A-C, the support bracket 8880 of the manual drive mechanism 8860 includes a wall 8881 and a mounting flange 8882. The wall 8881 defines a first aperture 8884 and a second aperture 8886. The support bracket is configured to locate or shield the engagement between the manual-drive-side coupling gear 8872 and the manual drive input gear member 8864. The first aperture 8884 is sized to receive the manual-drive-side coupling gear 8872. The second aperture 8886 is sized to receive the manual drive input gear member 8864. The two apertures merge together where the manual-drive-side coupling gear 8872 and the manual drive input gear member 8864 engage with one another. The mounting flange 8882 is configured to mount to the surface of upper chassis 8760. The upper chassis includes end stops 8888. The end stops 8888 protrude from the upper surface 8765 of the upper chassis 8760 (see e.g., FIG. 18). The end stops 8888 are located such that end stops 8877 contact the end stops 8888 when the manual drive coupling member 8890 is rotated sufficiently far in each direction.

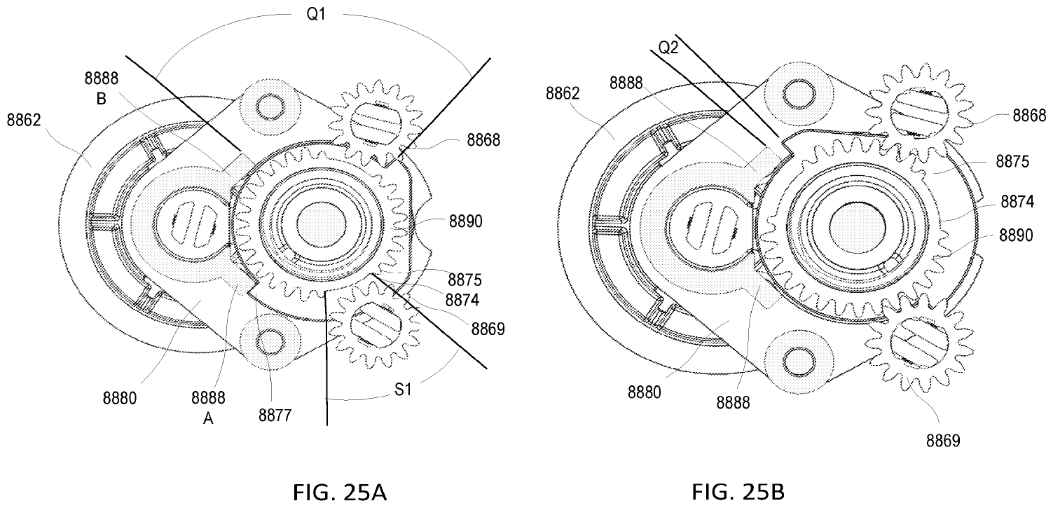

The manual drive coupling member 8890 is connected to the manual drive input member 8862 via engagement between the manual-drive-side coupling gear 8872 and the manual drive input gear 8864. As shown in FIGS. 24A-B, this engagement occurs within the support bracket 8880. Rotation of the manual input device member 8863 (about the first spindle 8766) causes rotation of the manual-drive-side coupling gear 8872 (about the second spindle 8767). The manual drive coupling member 8890 is selectably connected to the first capstan gear 8868 and the second capstan gear 8869. In a first state, the non-engagement portions 8874 of the manual drive coupling member 8890 are rotationally aligned with the teeth of the first capstan gear 8868 and the second capstan gear 8869 (see e.g., FIGS. 24A and 25A). In the first state, the manual drive coupling member 8890 is disengaged from the capstan gears. Thus, rotation of the first capstan gear 8868 or the second capstan gear 8869 does not result in any movement of the manual drive coupling member 8890 (or the manual drive input member 8862). In a second state, the engagement portion 8878 (e.g., teeth) of the manual drive coupling member 8890 are meshed with the teeth of the first capstan gear 8868 and the second capstan gear 8869. In this position, the teeth of the manual drive coupling member 8890 and the teeth of the capstan gears engage with one another, transferring torque therebetween (see e.g., FIGS. 24B and 25B). The manual drive coupling member 8890 transmits the input force from the manual drive input member 8862 to both the first capstan 8710 and the second capstan 8720. Specifically, the first capstan gear 8868 is coupled to the first capstan 8710 and the second capstan gear 8869 is coupled to the second capstan 8720, causing torque from the manual drive mechanism 8860 to be directed into the first capstan 8710 and the second capstan 8720. This allows the capstans to direct the force via the cables 8420 and 8430 to the end effector 8460 to open the jaws, as discussed in more detail above.

As shown in FIGS. 24A-25B, the different states in which the manual drive mechanism 8860 operates is based on the rotation of the manual drive coupling member 8890. FIG. 25A shows the total angular range of rotation of the manual drive coupling member 8890 as Q1. This range is the range from end stop 8888A to end stop 8888B. In some embodiments, this range can include rotation from 15 degrees to 350 degrees. In some embodiments, this range can include rotation from 45 degrees to 270 degrees. In some embodiments, this range can include rotation from 60 degrees to 120 degrees. In some embodiments, this range can be about 90 degrees (see e.g., FIG. 25A). The first state can be a sub-range within the total range. As shown, the manual drive mechanism operates in the first state in any rotational range where the first capstan gear 8868 and the second capstan gear 8869 are located adjacent (i.e., rotationally aligned with) the non-engagement portions 8874 of the manual drive coupling member 8890. In some embodiments, this non-engagement range can include rotation from 5 degrees to 60 degrees. In some embodiments, this non-engagement range can include rotation from 15 degrees to 45 degrees. In some embodiments, this range can be about 30 degrees. Thus, within the non-engagement range (the first state shown in FIGS. 24A and 25A), rotation of the manual drive coupling member 8890 does not cause rotation of the capstans. Likewise, rotation of the capstans does not cause rotation of the manual drive input 8862. Outside of the non-engagement range (the second state shown in FIGS. 24B and 25B), the manual drive mechanism 8860 operates in the second state in which rotation of the manual drive coupling member 8890 causes rotation of the capstans. Likewise, rotation of the capstans causes rotation of the manual drive input 8862.

The manual drive mechanism 8860 also includes a biasing member 8876 that is configured to bias the manual drive coupling member 8890 back to the first state. As shown in FIG. 16, the biasing member 8876 is a spring positioned between the manual drive coupling member 8890 and the upper chassis 8760. The spring places a torque on the manual drive coupling member 8890 to urge rotation of the manual drive member 8890 back towards the first state, with the end stop 8877 positioned against the end stop 8888.

Because the manual drive coupling member 8890 is disengaged from the first capstan gear 8868 and the second capstan gear 8869 in the first state, the manual drive coupling member 8890 limits interference (e.g., back drive) from the first capstan 8710 and the second capstan 8720 to the manual drive input member 8862. This also allows the first capstan 8710 and the second capstan 8720 to operate independently of one another (e.g., they can rotate independently, sometimes in the same direction, sometimes in opposite directions of one another, and sometimes one can be stationary while the other rotates).

As discussed above, articulation of the end effector relies on cable 8420 extending between capstans 8710 and 8730. Each capstan can be rotated to place the cable 8420 in tension and cause the cable 8420 to move. The non-driving capstan is also under load to keep the cable in tension. Because in this embodiment the manual drive mechanism 8860 drives the capstans 8710 and 8720, the cables 8420 and 8430, respectively, can only be placed in tension in one direction. When the biasing member causes the manual drive mechanism 8860 to return to the first state, capstans 8710 and 8720) may rotate, but they would rotate in the opposite direction of tension on the cables 8420 and 8430, potentially causing slack to form in the cables. It is appreciated that in other embodiments, the cables can extend between two capstans that are both driven by the manual drive mechanism 8860.

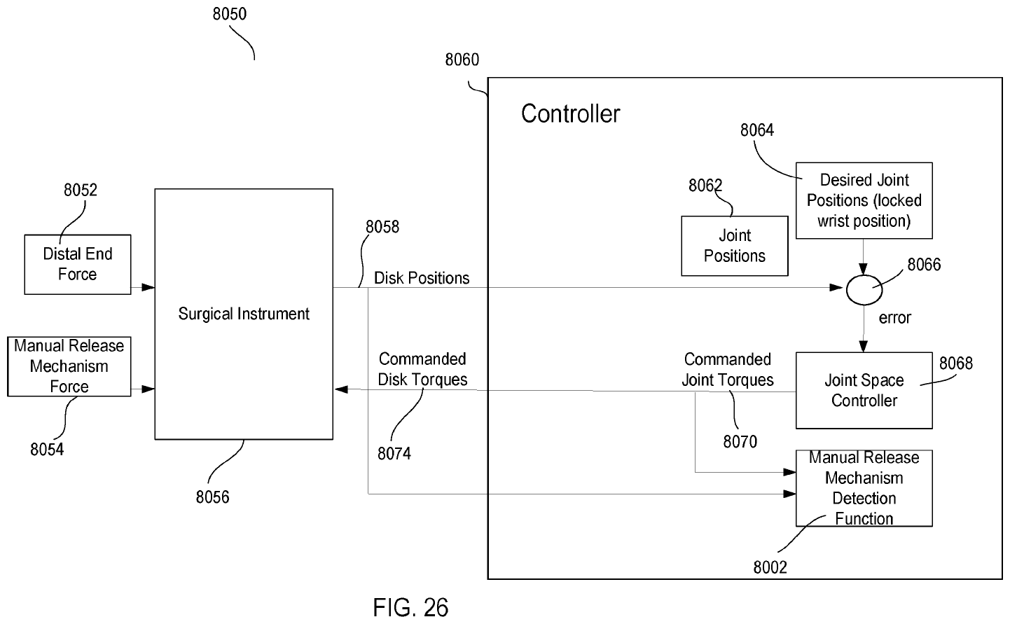

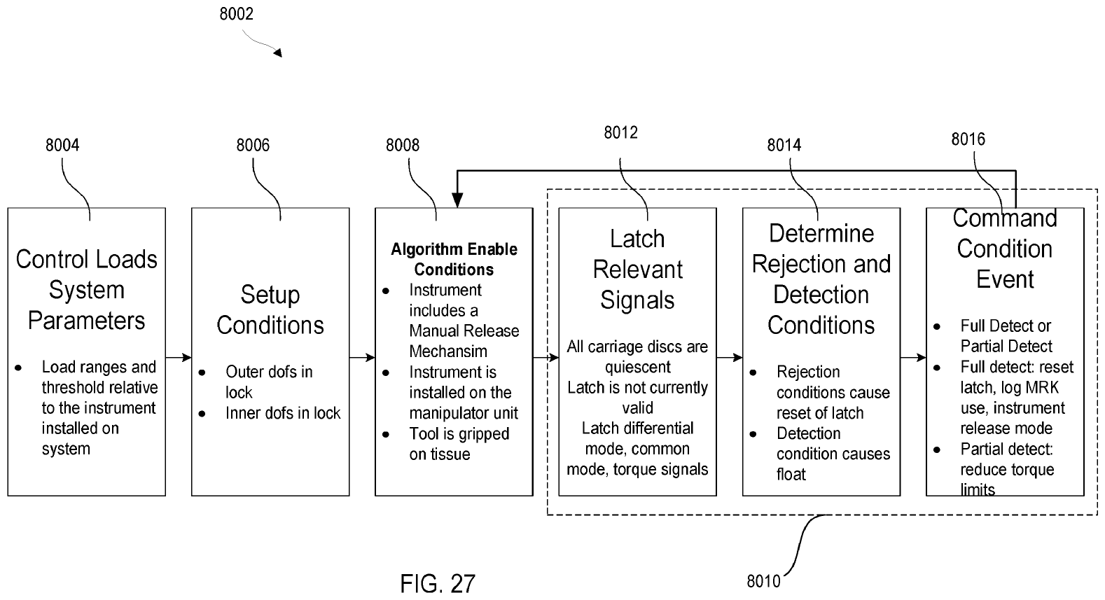

Selective engageability of the manual control of the surgical device 8400 allows for simplicity in operation of the device while providing redundancy of tool opening operation. By providing the manual input device 8863 and selective engageability as described above, the manual drive mechanism can be controlled and operated while the instrument is mounted on an associated teleoperated manipulator without the need for using a separate tool, instead being accessible and engaged by the hands of the user. Having the manual input device engage when desired removes undesirable distractions due to movement of the exterior features of the medical device 8400, thereby improving the usability of the medical device 8400 in a clinical setting. Having the manual input mechanism 8860 drive the two drivetrains (e.g., capstans 8710/8720, drive discs 8846, capstan gears 8869/8868, etc.) together, allows for the controller to detect use of the manual input mechanism 8860 by evaluating relative kinematic parameters of the two drivetrains. Example method steps for the evaluation are provided in more detail below with reference to FIGS. 26-27.

FIG. 26 is an example flow chart 8050 for control of the surgical system of FIG. 14-25B. As illustrated at 8056, the surgical instrument (e.g., the instrument 8400) can receive inputs from the commanded disk torques 8074 via the motors, distal end forces 8052 via the end effector and the manual release mechanism force 8054 via the manual release interface knob (e.g., the manual drive input member 8862). Information related to the surgical instrument can be communicated to the controller at 8060 via the disk positions of the motors at 8058. In some embodiments, this information regarding the disk positions of the motors can be received by manual release mechanism detection function. In some embodiments, disc positions and torques can be converted to the joint positions 8062 and/or torques used by the controller by applying a linear transform (not shown). In some embodiments, the information regarding the disk positions of the motors or the joint positions 8062 can be received by the joint position comparator at 8066. The desired joint position at 8064, which can be determined after input from distal end forces or use of the manual release mechanism is also received at the joint position comparator at 8066. The joint position controller at 8068 causes the actual joint position (disk position signal 8058) of all degrees of freedom to track the desired joint position from 8064. This difference or error between those two signals is minimized by modulating torque. In embodiments incorporating instrument locked control mode, the desired position of the joint is fixed. In some embodiments, it is the disk positions that are received as signals by the kinematic parameter signal function (e.g., 2910 of FIG. 5). In normal operation the system uses the information to control the joints by sending commanded joint torques 8070 and/or commanded disk torque signals 8074 to the instrument. Use of the manual release mechanism can be recognized at 8002 and is discussed in greater detail in FIG. 27.

FIG. 27 is a flow chart of a control method 8002 for a surgical system according to an embodiment. The method 8002 can, in an embodiment, be performed via the surgical system as described with reference to any of the systems shown in FIGS. 1-5 and 14-25B. However, it should be appreciated that in various embodiments, aspects of the method 8002 may be accomplished via additional embodiments of the surgical system or components thereof, such as instrument 8400. The method 8002 may be implemented, at least in part, via a controller (e.g., 2900 shown in FIG. 5). Instruction for operating the controller and the execution of the methods and processes 8002 can be stored in associated memory device(s) which are configured to perform the variety of computer implemented functions (e.g., performing the methods, steps, calculations and the like and storing relevant data as disclosed herein).

As depicted at 8004, the controller loads the system parameters that govern detection and operation of an instrument (e.g., instrument 8400) that includes a manual release mechanism (e.g., manual release mechanism 8860). The parameters include for example, the thresholds and ranges used by the algorithms to operate and detect the use of the manual release mechanism. As depicted at 8006, the system sets up for use. In embodiments, in which the manual release is detected from an instrument locked control mode, this can include locking the outer and inner degrees of freedom (dofs) of the instrument before the detection algorithm is instituted. In other embodiments, in which the manual release is detected from teleoperated input following control mode or limited following control mode, fewer or no dofs are locked. Other setup conditions can be included as well. As depicted at 8008, the system verifies that all algorithm conditions are enabled. For example, this can include verifying that the instrument includes a manual release mechanism, verifying that the instrument is installed on the manipulator unit, and that the instrument is in use and gripped on tissue. Upon verification of the enable conditions in 8008, the method 8010 for control of the manual release mechanism 8860 is active. This method can include all the method steps discussed in method 3010 (shown in FIG. 7). FIG. 27 illustrates additional detail of the method at 3010. For example, in this embodiment, signals from each of the drivetrains discussed above (e.g., part of drive system including capstans 8710, 8720) are latched at 8012. This can include having all the input discs quiescent and lacking a previous signal latch or the previous signal latch being invalid. In embodiments, where the manual release is operated have the system is in the instrument locked control mode, the two signals are received from locked inner and out degrees of freedom (dofs). In other embodiments, such as when the controller operates the actuator system (e.g., the manipulator unit 2200) in a teleoperated input following control mode, the controller receives the two signals corresponding with the two drive trains and latches the signals. This can also include comparing the first and second signals, calculating the differential mode and common mode, and latching the signals. Torque signals can also be latched at this point in the process.

As depicted at 8014, the latched signals can be checked against the defined ranges to detect usage of the manual release mechanism 8860 or determine that the manual release mechanism 8860 is not in use. If the manual release mechanism 8860 is not in use the condition is rejected and the latch is reset at 8016 back to 8008 and the instrument locked mode (or other mode as applicable) is continued for continued use of the system. If the manual release mechanism 8860 is in use it is determined if there is a full detect (such as a clear use of the manual release mechanism 8860) or a partial detect, (such as a questionable use of the manual release mechanism 8860). If there is a full detect (e.g., both common mode and differential mode indicate use) then the system is placed in instrument release mode and the use of the manual release mechanism 8860 is logged. If there is a partial detect (e.g., only one of the common mode and differential mode indicate use) then the system reduces the torque limits on the drive motors. In such an embodiment, more inclusive thresholds/ranges can be used for a partial detection. Such an implementation could include only looking at either common mode or differential mode meeting the requirement.

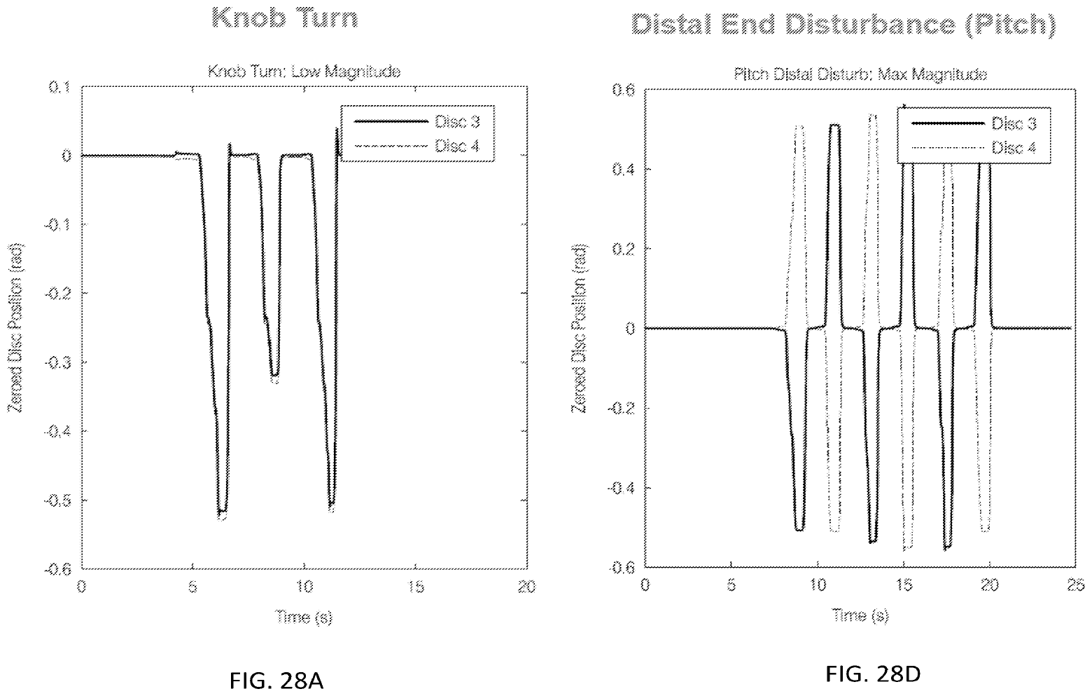

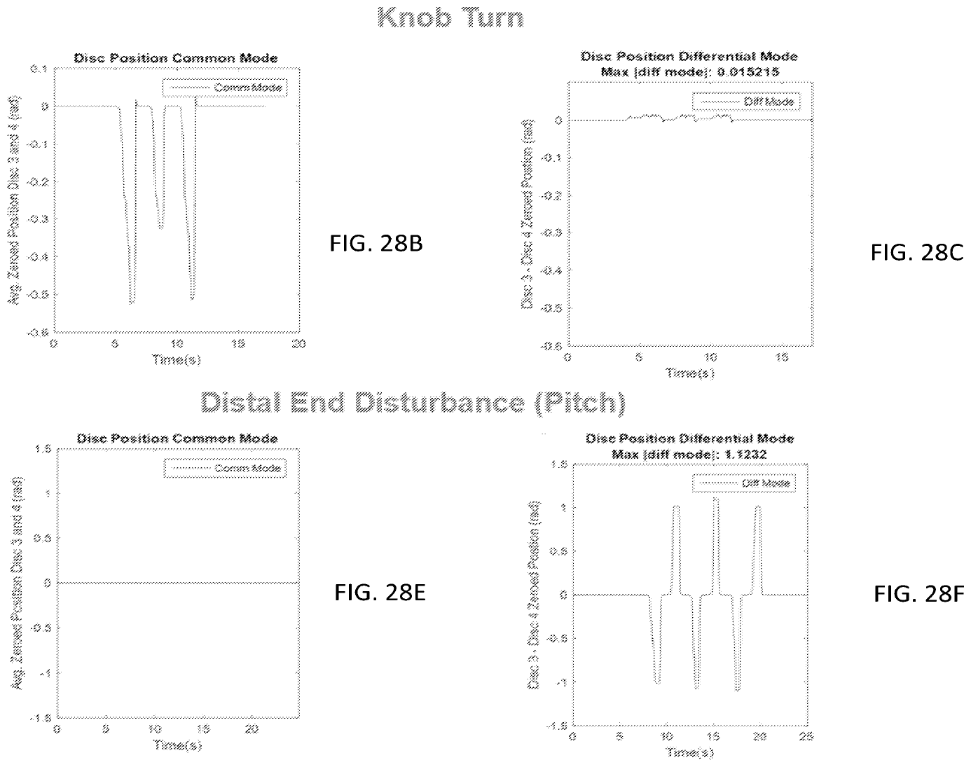

FIGS. 28A-F show examples of signals and signal processing for determining control of the surgical system 3000 (or any of the surgical systems described herein). FIG. 28A is a graph of a first signal and a second signal received from a first drive motor and a second drive motor respectively in the surgical system utilizing the instrument 8400 of FIG. 14 in response to the use of a manual input in accordance with one operational example. As shown, the signals from the motors related to the kinematic parameters of disks 3 and 4 are so closely overlapped that they are indistinguishable in the graph. FIG. 28B is a graph of the common mode of the signals in FIG. 28A. The common mode graph substantially matches the graph of FIG. 28A indicating the two signals in graph 28A substantially match. FIG. 28C is a graph of the differential mode of the two signals of 28A, the graph of FIG. 28C closely follows along 0 (with a max deviation off zero of 0.015215) indicating again that the two signals of graph 28A closely match. In this example, it is clear that the knob of the manual release mechanism 8860 has been turned causing the two drivetrains to rotate together. Thus, the condition to stop the instrument locked control mode and cause the instrument release mode is present.

FIG. 28D is a graph of a third signal and a fourth signal received from a first drive motor and a second drive motor respectively in the surgical system utilizing the instrument 8400 of FIG. 14 in response to an end effector disturbance in accordance with one operational example. Here, the first signal and the second signal are mirror images of each other indicating that disk 3 and disk 4 are rotating in opposite directions and not rotating together. FIG. 28E is a graph of the common mode of the signals in FIG. 28D. This graph shows the common mode to be zero. FIG. 28F is a graph of the differential mode of the signals of 28D. This graph shows the differential to have significant spikes to a max of twice the height of the signal spikes. This would indicate that the knob for the manual release mechanism 8860 has not been used. Indeed, these spikes are caused by the end effector having its pitch moved by the tissue. Thus, the system would remain in the instrument locked control mode.

FIG. 29A is an illustration of a portion of a surgical instrument 6400 according to an embodiment. The surgical instrument includes a mechanical structure 6700 having a manual drive mechanism 6860. As discussed in more detail below the manual drive mechanism 6860 drives a powertrain that includes a tool drive member 6710, gear 6868, and input disk 6846. These powertrain components are also variously referred to as movable components. A second powertrain is also included having tool drive member 6720, and input disk 6847.

As shown in FIG. 29A, the manual drive mechanism 6860 includes a manual input device 6863, signature generating mechanism 6862, and manual input gear 6890. The first powertrain can be driven by electromechanical drive or the manual drive mechanism 6860. For example, a motor can engage with input disk 6846 via a drive disk. Due to the engagement between the motor and the powertrain and the manual drive mechanism 6860 and the powertrain, the manual drive mechanism 6860 is also sufficiently mechanically coupled with the motor such that the manual drive mechanism 6860 drives the motor. Because of this coupling, the motor can produce a signal that reflects the signature produced by the signature generating mechanism 6862. This allows the controller 6900 to recognize use of the manual drive knob 6863 is in use and changes operation modes to accommodate the use of the manual drive knob 6863.

The manual drive mechanism 6860 includes manual input device 6863, a manual drive input member 6864, a signature generating mechanism 6862, and a manual drive coupling member 6890. The manual drive input member 6864 is mechanically connected to the manual input device 6863. The manual input device 6863 includes a portion that is exposed to the exterior of the medical device 6400. The user can engage the manual input device 6863 and manipulate the manual drive mechanism 6860, thereby manipulating the end effector. The exposed portion of the manual input device 6863 can include a rotatable knob that receive the user's input force and allows the manual drive mechanism 6860 to translate the user's input motion to an input on the tool drive members 6710 and thereby manipulate the end effector.

The manual drive mechanism 6860 also can produce a signature detectable via the electromechanically drive 6310 via the signature generating mechanism 6862. The signature generating mechanism 6862, shown in FIG. 29B, provides a distinctive signal to the sensor readable by the controller to indicate that the manual drive mechanism 6860 is in use. To do this, the signature generating mechanism 6862 produces a distinctive output of a kinematic parameter that is unlikely to be reproduced by feedback from the end effector. For example, the signature generating mechanism 6862 can include a clutch mechanism. In some embodiment, the clutch mechanism includes an inner clutch member 6892 attached to the drive coupling member 6890. The clutch mechanism can include an outer clutch member 6865 coupled to the manual input device knob 6863. The internal member 6892 can include teeth 6893 that are flexibly engaged with teeth 6867 on the outer member 6865. The flexible engagement between the teeth can cause the torque on or the position of the tool drive member 6710 to vary in a defined or otherwise known manner. As the torque on the manual input device knob 6863 increases, the teeth 6893 will flex and slip on the teeth 6867 past a certain torque threshold. This will cause movement then slippage at a constate rate related to the spacing of the teeth. These resulting torque or position spikes can then communicate to controller by receiving kinematic parameter values from the sensor.

FIG. 30 is an illustration of a portion of a surgical instrument 7400 according to an embodiment. The surgical instrument includes a mechanical structure 7700 having a manual drive mechanism 7860. As discussed in more detail below the manual drive mechanism 7860 drives a powertrain that includes one or more of a tool drive member 7710 and input disk 7847. While shown in FIG. 30 with a single powertrain connected to the manual drive mechanism 7860, multiple powertrains can be included (e.g., capstan 720). The powertrain can also be driven by a motor.

The manual drive mechanism 7860 includes manual input device knob 7863, a switch 7891, and a manual drive coupling member 7890. The manual input device knob 7863 includes a portion that is exposed to the exterior of the medical instrument 7400. The user can engage the manual input device 7863 and manipulate the manual drive mechanism 7860, thereby manipulating the end effector. The switch 7891 of the manual drive mechanism 7860 is configured to detect a kinematic parameter of the manual drive mechanism 7860. For example, the switch 7891 detects a change in position (e.g., angular orientation, transitional movement, etc.). The switch is triggered by a change in angular orientation of the manual drive mechanism. While described herein as a switch, in other examples, the sensor can be a speed sensor, torque sensor, or similar suitable device.

The various conceptual disclosures as described herein can be applied to the examples and embodiments described herein. However, it is also appreciated that the conceptual disclosures described herein can also be applied to other structures based on the a person of ordinary skill I the arts understanding of the concepts in light of the disclosure herein. For example, the disclosure herein can also be applied to embodiments of manual release mechanisms described in U.S. Patent Provisional Application No. 63/251,416 entitled “Mechanisms for Manually Activated Tool Adjustment”, filed Oct. 6, 2021, which is hereby incorporated by reference in its entirety.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the tool members can be constructed from any material, such as medical grade stainless steel, nickel alloys, titanium alloys or the like. Further, any of the links, tool members, tension members, or components described herein can be constructed from multiple pieces that are later joined together. For example, in some embodiments, a link can be constructed by joining together separately constructed components. In other embodiments however, any of the links, tool members, tension members, or components described herein can be monolithically constructed.

Although the instruments are generally shown as having an axis of rotation of the tool members (e.g., axis A1) that is normal to an axis of rotation of the wrist member (e.g., axis A2), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A surgical system, comprising:
an actuator system and a control system operatively coupled to the actuator system, the actuator system being operable to drive a first movable operating component and a second movable operating component;
wherein the control system includes a memory and instructions stored in the memory; and
wherein the instructions cause the control system to perform actions including:
commanding the actuator system to operate in a first control mode,
receiving a first signal containing a first kinematic parameter value of the first movable operating component,
receiving a second signal containing a second kinematic parameter value of the second movable operating component,
determining a compared value derived from both the first kinematic parameter value and the second kinematic parameter value, and
on a condition in which the compared value is within a defined value range, causing the control system to operate the actuator system in a second control mode.

2. The surgical system of claim 1, wherein:
the actions include:
on a condition in which the compared value is outside the defined value range, causing the control system to continue to operate the actuator system in the first control mode.

3. The surgical system of claim 1, wherein:
determining the compared value derived from both the first kinematic parameter value and the second kinematic parameter value includes combining the first kinematic parameter value and the second kinematic parameter value.

4. The surgical system of claim 1, wherein:
determining the compared value derived from both the first kinematic parameter value and the second kinematic parameter value includes determining a difference between the first kinematic parameter value and the second kinematic parameter value.

5. The surgical system of claim 1, wherein:
the compared value is a first compared value derived from both the first kinematic parameter value and the second kinematic parameter value according to a first calculation;
the defined value range is a first defined value range; and
the actions include:
determining a second compared value derived from both the first kinematic parameter value and the second kinematic parameter value according to a second calculation, and
on a condition in which the first compared value is within the first defined value range or the second compared value is within a second defined value range, causing the control system to operate the actuator system in the instrument second mode.

6. The surgical system of claim 5, wherein:
the first calculation includes combining the first kinematic parameter value and the second kinematic parameter value; and
the second calculation includes determining a difference between the first kinematic parameter value and the second kinematic parameter value.

7. The surgical system of claim 6, wherein:
the first defined value range is defined by values above a first threshold value above zero; and
the second defined value range is defined between zero and a second threshold value.

8. The surgical system of claim 1, wherein:
the first kinematic parameter value corresponds to a first kinematic parameter, the first kinematic parameter being one of a torque of the first movable operating component, a speed of the first movable operating component, or a position of the first movable operating component; and
the second kinematic parameter value corresponds to a second kinematic parameter, the second kinematic parameter being one of a torque of the second movable operating component, a speed of the second movable operating component, or a position of the second movable operating component.

9. The surgical system of claim 8, wherein:
the first kinematic parameter value is a moving average value of the first signal over time; and
the second kinematic parameter value is a moving average value of the second signal over time.

10. The surgical system of claim 1, wherein:
the first kinematic parameter value is associated with a position of the first movable operating component;
the second kinematic parameter value is associated with a position of the second movable operating component; and
the actions include:
latching the first kinematic parameter value and the second kinematic parameter value to determine a baseline prior to determining the compared value relative to the baseline.

11-13. (canceled)

14. The surgical system of claim 1, wherein:
the first mode is a system locked mode; and
the second mode is an instrument release mode.

15. The surgical system of claim 1, wherein:
the first mode is a teleoperated input following control mode; and
the second mode is an instrument release mode.

16-27. (canceled)

28. A surgical system comprising:
an actuator system and a control system operatively coupled to the actuator system, the actuator system being operable to drive a movable operating component;
wherein the control system includes a memory and instructions stored in the memory; and
wherein the instructions cause the control system to perform actions including:
commanding the actuator system to operate in a first control mode,
receiving one or more kinematic parameter values of the movable operating component,
on a first condition in which the one or more kinematic parameter values of the movable operating component is outside a defined range, causing the control system to continue to operate the actuator system in the first control mode, and
on a second condition in which the one or more kinematic parameter values of the movable operating component is within the defined range, causing the control system to operate the actuator system in a second control mode.

29. The surgical system of claim 28, wherein:
the surgical system includes a sensor and a manual control input;
the one or more kinematic parameter values of the movable operating component is based on information from the sensor and is associated with a state change of the manual control input; and
the manual control input is operable to drive the movable operating component.

30. The surgical system of claim 29, wherein:
the sensor is a switch that is actuated in response to movement of the manual control input.

31. The surgical system of claim 26, wherein:
the one or more kinematic parameter values is part of a plurality of kinematic parameter values of the movable operating component; and
the actions include:
receiving the plurality of kinematic parameter values, and
determining if the plurality of kinematic parameter values are associated with surgical instrument end effector movement driven by a manual input device.

32. The surgical system of claim 31, wherein:
the defined one or more kinematic parameter is a repeating pattern; and
the plurality of kinematic parameter values correspond to the repeating pattern.

33. The surgical system of claim 28, wherein:
the surgical system includes a manual control input;
the manual control input is operable to drive the movable operating component; and
the one or more kinematic parameter values are associated with a kinematic state change of the manual control input.

34. The surgical system of claim 28, wherein:
the surgical system includes a manual control input;
the manual control input is operable to drive the movable operating component; and
the one or more kinematic parameter values are associated with a kinematic state change of the movable operating component driven by the manual control input.

35. The surgical system of claim 28, wherein:
the surgical system includes a manual control input;
the manual control input is operable to drive the movable operating component; and
the one or more kinematic parameter values are associated with position, orientation, torque, speed, linear velocity, or angular velocity of the movable operating component driven by the manual control input.

36. The surgical system of claim 35, wherein:
the one or more kinematic parameter values include a repeating pattern associated with the position, orientation, torque, speed, linear velocity, or angular velocity of the movable operating component.

37. The surgical system of claim 28, wherein:
the movable operating component is a first movable operating component
the surgical system includes a second movable operating component;
the one or more kinematic parameter values includes a first plurality of kinematic parameter values of the first movable operating component and a second plurality of kinematic parameter values of the second movable operating component; and
the first plurality of kinematic parameter values and the second plurality of kinematic parameter values are associated with a kinematic state change of a manual control input operable to drive the first movable operating component and the second movable operating component.

38-50. (canceled)

51. The surgical system of claim 28, wherein:
the first mode is a system locked control mode; and
the second mode is an instrument release control mode.

52. The surgical system of claim 28, wherein:
the first mode is a teleoperated input following control mode; and
the second mode is an instrument release control mode.

53-71. (canceled)
